(19) United States
(12) Patent Application Publication
(10) Pub. No.: US 2011/0119785 A1
(43) Pub. Date: May 19, 2011

(54) NUCLEOTIDE SEQUENCES AND CORRESPONDING POLYPEPTIDES CONFERRING MODULATED GROWTH RATE AND BIOMASS IN PLANTS GROWN IN SALINE AND OXIDATIVE CONDITIONS

(75) Inventors: Fasong Zhou, Fremont, CA (US); Kerstin Piccolo, Woodland Hills, CA (US); Julissa Sosa, Northridge, CA (US)

(73) Assignee: CERES, INC., Thousand Oaks, CA (US)

(21) Appl. No.: 12/922,143

(22) PCT Filed: Mar. 12, 2009
(86) PCT No.: PCT/US09/37025

§ 371 Date: Feb. 1, 2011

Related U.S. Application Data
(60) Provisional application No. 61/036,396, filed on Mar. 13, 2008.

(51) Int. Cl. A01H 1/02; C12N 5/10; A01H 1/00; A01H 5/00; A01H 5/04; C07H 21/04; C12Q 1/68

(52) U.S. Cl. 800/275; 435/419; 800/278; 800/295; 800/298; 800/320; 800/320.1; 800/320.3; 800/312; 800/314; 800/320.2; 800/322; 536/23.6; 435/6; 800/260

(57) ABSTRACT

The present invention relates to isolated nucleic acid molecules and their corresponding encoded polypeptides able confer the trait of improved plant size, vegetative growth, growth rate, seedling vigor and/or biomass in plants challenged with saline and/or oxidative stress conditions. The present invention further relates to the use of these nucleic acid molecules and polypeptides in making transgenic plants, plant cells, plant materials or seeds of a plant having plant size, vegetative growth, growth rate, seedling vigor and/or biomass that are improved in saline and/or oxidative stress conditions with respect to wild-type plants grown under similar conditions.

### CROSS-REFERENCE TO RELATED APPLICATIONS

The present application claims priority to prior U.S. provisional application Ser. No. 61/036,396, filed on Mar. 13, 2008, the entire contents of which are hereby incorporated by reference.

### INCORPORATION-BY-REFERENCE OF SEQUENCE LISTING OR TABLE

The material in the accompanying sequence listing is hereby incorporated by reference into this application in its entirety. The accompanying file, named 2009_{—}03_{—}05_{—}2750_{—}1711PWO1_SequenceListingMSDOS_Format.txt was created on Mar. 3, 2009 and is 3,357 KB. The file can be accessed using Microsoft Word on a computer that uses Windows OS.

### TECHNICAL FIELD

The present invention relates to isolated nucleic acid molecules and their corresponding encoded polypeptides able to enhance plant growth under saline and/or oxidative stress conditions. The present invention further relates to using the nucleic acid molecules and polypeptides to make transgenic plants, plant cells, plant materials or seeds of a plant having improved growth rate, vegetative growth, seedling vigor and/or biomass under saline and/or oxidative stress conditions as compared to wild-type plants grown under similar conditions.

### BACKGROUND

Plants specifically improved for agriculture, horticulture, biomass conversion, and other industries (e.g. biofuel, paper industry, plants as production factories for proteins or other compounds) can be obtained using molecular technologies. As an example, great agronomic value can result from enhancing plant growth in and/or tolerance to saline and/or oxidative stress conditions.

### Salinity

A wide variety of agriculturally important plant species demonstrate significant sensitivity to saline stress conditions. Upon salt concentration exceeding a relatively low threshold, many plants suffer from stunted growth, necrosis, and death that results in an overall stunted appearance and reduced yields of plant material, seeds, fruit and other valuable products. Physiologically, plants challenged with salinity experience disruption in ion and water homeostasis, inhibition of metabolism, and damage to cellular membranes that result in developmental arrest and cell death (Huh et al. (2002) Plant J, 29(5):649-59).

In many of the world's most productive agricultural regions, agricultural activities themselves lead to increased water and soil salinity, which threatens their sustained productivity. One example is crop irrigation in arid regions that have abundant sunlight. After irrigation water is applied to cropland, it is removed by the processes of evaporation and transpiration. While these processes remove water from the soil, they leave behind dissolved salts carried in irrigation water. Consequently, soil and groundwater salt concentrations build over time, rendering the land and shallow groundwater saline and thus damaging to crops.

In addition to human activities, natural geological processes have created vast tracts of saline land that would be highly productive if not saline. In total, approximately 20% of the irrigated lands are negatively affected by salinity (Yamaguchi and Blumwald, 2005, Trends in Plant Science, 10: 615-620). For these and other reasons, it is of great interest and importance to identify genes that confer improved salt tolerance characteristics to thereby enable one to create transgenic plants (such as crop plants) with enhanced growth and/or productivity characteristics in saline conditions.

Despite this progress, today there continues to be a great need for generally applicable processes that improve forest or agricultural plant growth to suit particular needs depending on specific environmental conditions. To this end, the present invention is directed to advantageously manipulating plant tolerance to salinity in order to maximize the benefits of various crops depending on the benefit sought, and is characterized by expression of recombinant DNA molecules in plants. These molecules may be from the plant itself, and simply expressed at a higher or lower level, or the molecules may be from different species.

### Oxidative Stress

Plants lead a sessile lifestyle and so are generally destined to reside where their seed germinates. Consequently, they can be exposed to unfavorable environmental conditions arising from weather, pollution and location. Stress conditions, such as extremes in temperature, drought and desiccation, salinity, soil nutrient content, heavy metals, UV radiation, pollutants such as ozone and SO₂, mechanical stress, high light and pathogen attack, have a large impact on plant growth and development. These types of stress exposure induce formation of toxic oxygen species, which are generated in all aerobic cells and are associated with oxidative damage at the cellular level. Several published reports have characterized toxic oxygen species generation and the subsequent oxidative damage caused by abiotic stresses (see Larkindale and Knight (2002); Borsani et al. (2001); Lee et al (2004); Aroca et al (2005); Luna et al (2005); and Noctor et al (2002)).

The toxic oxygen species are referred to as reactive oxygen species (ROS), reactive oxygen intermediates (ROI) or activated oxygen species (AOS) and are partially reduced or activated derivatives of oxygen. ROS/ROI/AOS include the oxygen-centered superoxide (O₂) and hydroxyl (.OH) free radicals as well as hydrogen peroxide (H₂O₂), nitric oxide (NO) and O₂. These oxygen species are generated as byproducts from reactions that occur during photosynthesis, respiration and photorespiration, and are predominantly formed in the chloroplasts, mitochondria, endoplasmic reticulum, microbodies (e.g. peroxisomes and glyoxysomes), plasma membranes and cell walls. While the toxicity of O₂⁻ and H₂O₂ themselves is relatively low, their metal-dependent conversion to highly toxic .OH is thought to be responsible for the majority of the biological damage associated with these molecules.

Oxidative stress damages cell structure and affects cell metabolism and catabolism. Membrane lipids are subject to oxidation by ROS/ROI/AOS, resulting in accumulation of high molecular weight, cross-linked fatty acids and phospholipids. Oxidative attack on proteins results in site-specific amino acid modifications, fragmentation of the peptide chain, aggregation of cross-linked reaction products, altered electrical charge and increased susceptibility to proteolysis, all of which frequently leads to elimination of enzyme activity. ROS/ROI/AOS that generate oxygen free radicals, such as ionizing radiation, also induce numerous lesions in DNA at both the sugar and base moieties which cause deletions, mutation and other lethal genetic effects such as base degradation, single strand breakage and cross-linking to proteins. Morphologically, the adverse effects of high levels of ROS accumulation are manifested as stunted growth and necrotic lesions.

Although capable of producing damage, ROS/ROI/AOS are also key regulators of metabolic and defense pathways, playing roles as signaling or secondary messenger molecules. For example, pathogen-induced ROS/ROI/AOS production is critical in disease resistance where these molecules are involved at three different levels: penetration resistance, hypersensitive response (HR) and systemic acquired resistance (Levine et al. (1994); Lamb and Dixon (1997); Zhou et al. (2000); Aviv et al. (2002)). In penetration resistance, ROS/ROI/AOS function by reinforcing cell walls through polyphenolic cross-linking. With respect to hypersensitive response, H₂O₂ is an active signaling molecule whose effect is dose dependent. At high dosages, H₂O₂ triggers hypersensitive cell death and thus restricts the pathogen to local infection sites (Lamb and Dixon (1997)) while low dosages block cell cycle progression (Reichheld et al. (1999)) and signal secondary wall differentiation (Potikha et al. (1999)). Lastly, ROS/ROI/AOS molecules play a role in broad-spectrum systemic acquired disease resistance by triggering micro-HR systematically after the first pathogen inoculation.

In the signal cascades leading to oxidative stress, salicylic acid (SA) has been identified as an important signaling molecule to mediate ROS/ROI/AOS accumulation in various stress conditions, such as salt and osmotic stress (Borsani et al. (2001)), drought (Senaratna et al. (2000)), heat (Dat et al. (1998)), cold (Scott et al. (2004)), UV-light (Surplus et al. (1998)), paraquat (Kim et al. (2003)) and disease resistance against different pathogens (Zhou et al. (2004)). High levels of SA induce H₂O₂ production as well as cell death.

Several signaling components required for SA-mediated ROS/ROI/AOS accumulation and gene expression have been characterized. For example, NPR1 is required for SA-induced PR gene expression and disease resistance (Cao et al. (1994)). The mutations in eds1 and eds5 block SA-mediated signaling and enhance disease susceptibility (Rusterucci et al. (2001)). Over-expression of NahG in various plant species also suppresses SA-induced responses to both abiotic and biotic stresses (Delaney et al. (1994)). Recently, Scott and colleagues (2004) reported that chilling treatment induced accumulation of SA in Arabidopsis and the degradation of SA by overexpression of NahG enhanced cold tolerance in a transgenic plant.

SA, as a phytohormone, also promotes early flowering (Martinez et al. (2004)). SA at various levels may play different roles in plant growth and stress responses. However, most of the time, the increased tolerance to high levels of SA appears to be beneficial, since it reduces the side effects of SA accumulation while stimulating SA-mediated stress responses.

Similarly, NO is capable of generating ROS/ROI/AOS and is a plant signaling molecule involved in the regulation of seed germination, stomatal closure (Mata and Lamattina (2001); Desikan et al. (2002)), flowering time (He et al. (2004)), antioxidant reactions to suppress cell death (Beligni et al. (2002)) and tolerance to biotic and abiotic stress conditions (Mata and Lamattina (2001)). While the effects of NO can be mimicked through the application of sodium nitroprusside (SNP), endogenous NO production in plants results from the activity of a nitric oxide synthase that uses L-arginine (Guo et al. (2003)) as well as nitrate reductase-mediated reactions (Desikan et al (2002)). NO can react with redox centers in proteins and membranes, thereby causing cell damage and inducing cell death.

In order to control the two-fold nature of ROS/ROI/AOS molecules, plants have developed a sophisticated regulatory system which involves both production and scavenging of ROS/ROI/AOS in cells. During normal growth and development, this pathway monitors the level of ROS/ROI/AOS produced by metabolism and controls the expression and activity of ROS/ROI/AOS scavenging pathways. The major ROS/ROI/AOS scavenging mechanisms include the action of the superoxide dismutase (SOD), ascorbate perioxidase (APX) and catalase (CAT) enzymes as well as nonenzymatic components such as ascorbic acid, α-tocopherol and glutathione.

The antioxidant enzymes are believed to be critical components in preventing oxidative stress, in part because pretreatment of plants with one form of stress, and which induces expression of these enzymes, can increase tolerance for a different stress (cross-tolerance) Allen (1995)). In addition, plant lines selected for resistance to herbicides that function by inducing ROS/R01/AOS generally have increased levels of one or more of these antioxidant enzymes and also exhibit cross-tolerance (Gressel and Galun (1994)).

Plant development and yield depend on the ability of the plant to manage oxidative stress, whether it is via the signaling or the scavenging pathways. Consequently, improvements in a plant's ability to withstand oxidative stress, or to obtain a higher degree of cross-tolerance once oxidative stress has been experienced, has significant value in agriculture. The sequences and methods of the invention provide the means by which tolerance to oxidative stress can be improved, either via the signaling or the scavenging pathways.

### Plant Improvement

The availability and sustainability of a stream of food and feed for people and domesticated animals has been a high priority throughout the history of human civilization and lies at the origin of agriculture. Specialists and researchers in the fields of agronomy science, agriculture, crop science, horticulture, and forest science are even today constantly striving to find and produce plants with an increased growth potential to feed an increasing world population and to guarantee a supply of reproducible raw materials. The robust level of research in these fields of science indicates the level of importance leaders in every geographic environment and climate around the world place on providing sustainable sources of food, feed and energy.

Manipulation of crop performance has been accomplished conventionally for centuries through selection and plant breeding. The breeding process is, however, both time-consuming and labor-intensive. Furthermore, appropriate breeding programs must be specially designed for each relevant plant species.

On the other hand, great progress has been made in using molecular genetic approaches to manipulate plants to provide better crops. Through the introduction and expression of recombinant nucleic acid molecules in plants, researchers are now poised to provide the community with plant species tailored to grow more efficiently and yield more product despite suboptimal geographic and/or climatic environments. These new approaches have the additional advantage of not being limited to one plant species, but instead being applicable to multiple different plant species (Zhang et al. (2004) Plant Physiol. 135:615; Zhang et al. (2001) Proc. Natl. Acad. Sci. USA 98:12832).

### SUMMARY

This document provides methods and materials related to plants having modulated levels of tolerance to salinity and/or oxidative stress. For example, this document provides transgenic plants and plant cells having increased levels of tolerance to salinity and/or oxidative stress, nucleic acids used to generate transgenic plants and plant cells having increased levels of tolerance to salinity and/or oxidative stress, and methods for making plants and plant cells having increased levels of tolerance to salinity and/or oxidative stress. Such plants and plant cells provide the opportunity to produce crops or plants under saline and/or oxidative stress conditions without stunted growth and diminished yields. Increased levels of tolerance to salinity and/or oxidative stress may be useful to produce biomass which may be converted to a liquid fuel or other chemicals and/or to produce food and feed on land that is currently marginally productive, resulting in an overall expansion of arable land.

Methods of producing a plant and/or plant tissue are provided herein. In one aspect, a method of producing a plant and/or plant tissue having increased tolerance to salinity and/or increased tolerance to oxidative stress comprises growing a plant cell comprising an exogenous nucleic acid. The exogenous nucleic acid comprises a regulatory region operably linked to a nucleotide sequence encoding a polypeptide. The amino acid sequence of the encoded polypeptide has a Hidden Markov Model (HMM) bit score greater than about 15 using an HMM generated from the amino acid sequences depicted in one of FIGS. 1 to 16. The plant and/or plant tissue has a difference in the level of tolerance to salinity and/or oxidative stress as compared to the corresponding level in tolerance to salinity and/or oxidative stress of a control plant that does not comprise the exogenous nucleic acid. In some embodiments the amino acid sequence of the polypeptide has an HMM bit score greater than about 170 using an HMM generated from the amino acid sequences depicted in FIG. 1. In some embodiments the amino acid sequence of the polypeptide has an HMM bit score greater than about 255 using an HMM generated from the amino acid sequences depicted in FIG. 2. In some embodiments the amino acid sequence of the polypeptide has an HMM bit score greater than about 65 using an HMM generated from the amino acid sequences depicted in FIG. 3. In some embodiments the amino acid sequence of the polypeptide has an HMM bit score greater than about 175 using an HMM generated from the amino acid sequences depicted in FIG. 4. In some embodiments the amino acid sequence of the polypeptide has an HMM bit score greater than about 1095 using an HMM generated from the amino acid sequences depicted in FIG. 5. In some embodiments the amino acid sequence of the polypeptide has an HMM bit score greater than about 535 using an HMM generated from the amino acid sequences depicted in FIG. 6. In some embodiments the amino acid sequence of the polypeptide has an HMM bit score greater than about 120 using an HMM generated from the amino acid sequences depicted in FIG. 7. In some embodiments the amino acid sequence of the polypeptide has an HMM bit score greater than about 455 using an HMM generated from the amino acid sequences depicted in FIG. 8. In some embodiments the amino acid sequence of the polypeptide has an HMM bit score greater than about 180 using an HMM generated from the amino acid sequences depicted in FIG. 9. In some embodiments the amino acid sequence of the polypeptide has an HMM bit score greater than about 110 using an HMM generated from the amino acid sequences depicted in FIG. 10. In some embodiments the amino acid sequence of the polypeptide has an HMM bit score greater than about 75 using an HMM generated from the amino acid sequences depicted in FIG. 11. In some embodiments the amino acid sequence of the polypeptide has an HMM bit score greater than about 15 using an HMM generated from the amino acid sequences depicted in FIG. 12. In some embodiments the amino acid sequence of the polypeptide has an HMM bit score greater than about 70 using an HMM generated from the amino acid sequences depicted in FIG. 13. In some embodiments the amino acid sequence of the polypeptide has an HMM bit score greater than about 50 using an HMM generated from the amino acid sequences depicted in FIG. 14. In some embodiments the amino acid sequence of the polypeptide has an HMM bit score greater than about 300 using an HMM generated from the amino acid sequences depicted in FIG. 15. In some embodiments the amino acid sequence of the polypeptide has an HMM bit score greater than about 25 using an HMM generated from the amino acid sequences depicted in FIG. 16.

In another aspect, a method comprises growing a plant cell comprising an exogenous nucleic acid. The exogenous nucleic acid comprises a regulatory region operably linked to a nucleotide sequence encoding a polypeptide having at least 80%, 85%, 90%, 95%, 97%, 98% or greater sequence identity to an amino acid sequence set forth in SEQ ID NOs: 2, 11, 102, 32, 138, 149, 441, 172, 764, 389, 316, 200, 697, 239, 892, 922, 693 or any of the amino acid sequences set forth in the sequence listing. A plant produced from the plant cell has a difference in the level of tolerance to salinity and/or oxidative stress as compared to the corresponding level in a control plant that does not comprise the exogenous nucleic acid.

In another aspect, a method comprises growing a plant cell comprising an exogenous nucleic acid. The exogenous nucleic acid comprises a regulatory region operably linked to a nucleotide sequence having at least 80%, 85%, 90%, 95%, 97%, 98% or greater sequence identity to at least a fragment of a nucleotide sequence set forth in SEQ ID NOs. 1, 10, 101, 31, 137, 148, 440, 171, 763, 388, 315, 199, 696, 238, 891, 921, 692 or a nucleotide sequence encoding any of the amino acid sequences set forth in the sequence listing. A plant and/or plant tissue produced from the plant cell has a difference in the level of salinity and/or oxidative stress tolerance as compared to the corresponding level in a control plant that does not comprise the exogenous nucleic acid.

Methods of modulating the level of salt tolerance and/or oxidative stress tolerance in a plant are provided herein. In one aspect, a method comprises introducing into a plant cell an exogenous nucleic acid, that comprises a regulatory region operably linked to a nucleotide sequence encoding a polypeptide. The HMM bit score of the amino acid sequence of the polypeptide is greater than 170, 255, 65, 175, 1095, 535, 120, 455, 180, 110, 75, 15, 70, 50, 300, or 25, using an HMM generated from the amino acid sequences depicted in one of FIGS. 1-16, respectively. A plant and/or plant tissue produced from the plant cell has a difference in the level of tolerance to salinity and/or oxidative stress as compared to the corresponding level in a control plant that does not comprise the exogenous nucleic acid.

In another aspect, a method comprises introducing into a plant cell an exogenous nucleic acid that comprises a regulatory region operably linked to a nucleotide sequence encoding a polypeptide having at least 80%, 85%, 90%, 95%, 97%, 98% or greater sequence identity to an amino acid sequence set forth in SEQ ID NOs: 2, 11, 102, 32, 138, 149, 441, 172, 764, 389, 316, 200, 697, 239, 892, 922, 693 or any of the amino acid sequences set forth in the sequence listing. A plant and/or plant tissue produced from the plant cell has a difference in the level of tolerance to salinity or oxidative stress as compared to the corresponding level in a control plant that does not comprise the exogenous nucleic acid.

In some embodiments, the methods comprise introducing into the plant cell an exogenous nucleic acid encoding polypeptides selected from the group consisting of SEQ ID NOs: 2, 11, 102, 32, 138, 149, 441, 172, 764, 389, 316, 200, 697, 239, 892, 922, 693 and any of the amino acid sequences set forth in the sequence listing. A plant and/or plant tissue produced from the plant cell has a difference in the level of tolerance to salinity as compared to the corresponding level in a control plant that does not comprise the exogenous nucleic acid. In some embodiments, the methods comprise introducing into the plant cell an exogenous nucleic acid encoding polypeptides selected from the group consisting of SEQ ID NO: 2, 11, 102, 32, 138, 149, 441, 172, 764, 389, 316, 200, 697, 239, 892, 922, 693 and any of the amino acid sequences set forth in the sequence listing, and a plant and/or plant tissue produced from the plant cell has a difference in the level of tolerance to oxidative stress as compared to the corresponding level in a control plant that does not comprise the exogenous nucleic acid.

In another aspect, a method comprises introducing into a plant cell an exogenous nucleic acid, that comprises a regulatory region operably linked to a nucleotide sequence having at least 80%, 85%, 90%, 95%, 97%, 98% or greater sequence identity to a nucleotide sequence, or a fragment thereof, set forth in SEQ ID NOs: 1, 10, 101, 31, 137, 148, 440, 171, 763, 388, 315, 199, 696, 238, 891, 921, 692, or to a nucleotide sequence encoding any of the amino acid sequences set forth in the sequence listing, or a fragment thereof. A plant and/or plant tissue produced from the plant cell has a difference in the level of tolerance to salinity or oxidative stress as compared to the corresponding level in a control plant that does not comprise the exogenous nucleic acid.

Plant cells comprising an exogenous nucleic acid are provided herein. In one aspect, the exogenous nucleic acid comprises a regulatory region operably linked to a nucleotide sequence encoding a polypeptide. The HMM bit score of the amino acid sequence of the polypeptide is greater than 170, 255, 65, 175, 1095, 535, 120, 455, 180, 110, 75, 15, 70, 50, 300, or 25, using an HMM based on the amino acid sequences depicted in one of FIGS. 1-16. The plant and/or plant tissue has a difference in the level of tolerance to salinity or oxidative stress as compared to the corresponding level in a control plant that does not comprise the exogenous nucleic acid. In another aspect, the exogenous nucleic acid comprises a regulatory region operably linked to a nucleotide sequence encoding a polypeptide having at least 80%, 85%, 90%, 95%, 97%, 98% or greater sequence identity to an amino acid sequence selected from the group consisting of SEQ ID NOs: 2, 11, 102, 32, 138, 149, 441, 172, 764, 389, 316, 200, 697, 239, 892, 922, 693, and any of the amino acid sequences set forth in the sequence listing. A plant and/or plant tissue produced from the plant cell has a difference in the level of tolerance to salinity or oxidative stress as compared to the corresponding level in a control plant that does not comprise the exogenous nucleic acid. In another aspect, the exogenous nucleic acid comprises a regulatory region operably linked to a nucleotide sequence having 80 percent or greater sequence identity to at least a fragment of a nucleotide sequence selected from the group consisting of SEQ ID Nos. 1, 10, 101, 31, 137, 148, 440, 171, 763, 388, 315, 199, 696, 238, 891, 921, 692, and to a nucleotide sequence encoding any of the amino acid sequences set forth in the sequence listing. A plant and/or plant tissue produced from the plant cell has a difference in the level of tolerance to salinity or oxidative stress as compared to the corresponding level in a control plant that does not comprise the exogenous nucleic acid. A transgenic plant comprising such a plant cell is also provided. In some embodiments, the transgenic plant is a member of a species selected from the group consisting of Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), or Pennisetum glaucum (pearl millet). Some embodiments are related to products comprising seed or vegetative tissue from transgenic plants as described above. Some seed products comprise embryonic tissue from transgenic plants as described above. Some embodiments relate to food or feed products from transgenic plants as described above.

In another aspect, an isolated nucleic acid comprises a nucleotide sequence encoding a polypeptide having at least 80%, 85%, 90%, 95%, 97%, 98% or greater sequence identity to the amino acid sequence set forth in SEQ ID Nos. 2, 11, 102, 32, 138, 149, 441, 172, 764, 389, 316, 200, 697, 239, 892, 922, 693, or any of the amino acid sequences set forth in the sequence listing.

In another aspect, an isolated nucleic acid comprises an isolated nucleic acid comprising a nucleotide sequence having at least 80%, 85%, 90%, 95%, 97%, 98% or greater sequence identity to the nucleotide sequence set forth in SEQ ID NO: 10, 12, 14, 16, 18, 20, 22, 24, 26, 28, 31, 34, 36, 39, 41, 43, 45, 52, 55, 57, 59, 61, 70, 72, 74, 76, 78, 80, 82, 84, 86, 88, 90, 92, 94, 96, 101, 105, 107, 109, 113, 121, 123, 126, 131, 133, 135, 139, 141, 145, 150, 152, 156, 158, 160, 166, 168, 170, 171, 173, 177, 181, 183, 185, 190, 193, 195, 197, 201, 203, 205, 207, 216, 218, 222, 224, 227, 229, 231, 234, 236, 240, 242, 244, 246, 248, 252, 254, 258, 260, 263, 265, 269, 274, 278, 280, 283, 285, 287, 289, 296, 298, 300, 303, 305, 307, 317, 322, 332, 334, 336, 338, 343, 345, 347, 349, 352, 354, 360, 362, 364, 366, 368, 376, 381, 383, 385, 391, 393, 397, 399, 404, 413, 415, 423, 425, 427, 429, 435, 443, 448, 455, 466, 476, 478, 483, 485, 495, 497, 499, 501, 510, 515, 521, 526, 528, 530, 535, 542, 545, 548, 554, 558, 561, 568, 570, 573, 575, 579, 581, 584, 587, 589, 596, 599, 602, 604, 607, 612, 619, 623, 630, 633, 634, 636, 638, 645, 647, 649, 652, 656, 658, 667, 670, 672, 674, 676, 679, 681, 683, 685, 687, 689, 691, 695, 698, 702, 708, 710, 713, 717, 719, 721, 729, 731, 734, 736, 738, 744, 748, 750, 754, 756, 758, 760, 768, 772, 778, 783, 787, 789, 791, 794, 799, 803, 811, 821, 823, 828, 830, 833, 835, 837, 839, 842, 844, 853, 859, 865, 873, 875, 877, 879, 881, 883, 885, 887, 889, 893, 895, 897, 900, 902, 904, 909, 910, 912, 917, 919, 921, 923, 925, 927, 933, 935, 937, 939, 941, 943, 945, 947, 949, 951, 953, 955, 957, 959, 961, 963, 965, 967, 969, 971, 973, 980, 984, 986, 988, 990, or 992, or an homolog thereof such as those identified in the sequence listing.

In another aspect, an isolated nucleic acid comprises an isolated nucleic acid comprising a nucleotide sequence encoding a polypeptide having 80% or greater sequence identity to the amino acid sequence set forth in SEQ ID NO: 11, 13, 15, 17, 19, 21, 23, 25, 27, 29, 32, 35, 37, 40, 42, 44, 46, 53, 56, 58, 60, 62, 71, 73, 75, 77, 79, 81, 83, 85, 87, 89, 91, 93, 95, 97, 102, 106, 108, 110, 114, 122, 124, 127, 132, 134, 136, 140, 142, 146, 151, 153, 157, 159, 161, 167, 169, 172, 174, 178, 182, 184, 186, 191, 194, 196, 198, 202, 204, 206, 208, 217, 219, 223, 225, 228, 230, 232, 235, 237, 241, 243, 245, 247, 249, 253, 255, 259, 261, 264, 266, 270, 275, 279, 281, 284, 286, 288, 290, 297, 299, 301, 304, 306, 308, 318, 323, 333, 335, 337, 339, 344, 346, 348, 350, 353, 355, 361, 363, 365, 367, 369, 377, 382, 384, 386, 392, 394, 398, 400, 405, 414, 416, 424, 426, 428, 430, 436, 444, 449, 456, 467, 477, 479, 484, 486, 496, 498, 500, 502, 511, 516, 522, 527, 529, 531, 536, 543, 546, 549, 555, 559, 562, 569, 571, 574, 576, 580, 582, 585, 588, 590, 597, 600, 603, 605, 608, 613, 620, 624, 631, 634, 635, 637, 639, 646, 648, 650, 653, 657, 659, 668, 671, 673, 675, 677, 680, 682, 684, 686, 688, 690, 692, 696, 699, 703, 709, 711, 714, 718, 720, 722, 730, 732, 735, 737, 739, 745, 749, 751, 755, 757, 759, 761, 769, 773, 779, 784, 788, 790, 792, 795, 800, 804, 812, 822, 824, 829, 831, 834, 836, 838, 840, 843, 845, 854, 860, 866, 874, 876, 878, 880, 882, 884, 886, 888, 890, 894, 896, 898, 901, 903, 905, 910, 911, 913, 918, 920, 922, 924, 926, 928, 934, 936, 938, 940, 942, 944, 946, 948, 950, 952, 954, 956, 958, 960, 962, 964, 966, 968, 970, 972, 974, 981, 985, 987, 989, 991, or 993, or an homolog thereof such as those identified in the sequence listing.

In another aspect, methods of identifying a genetic polymorphism associated with variation in the level of salinity and/or oxidative stress tolerance are provided. The methods include providing a population of plants, and determining whether one or more genetic polymorphisms in the population are genetically linked to the locus for a polypeptide selected from the group consisting of the polypeptides depicted in FIGS. 1-16, functional homologs thereof, and polypeptide sequences in the sequence listing. The correlation between variation in the level of salinity and/or oxidative stress tolerance in plants and/or plant tissues of the population and the presence of the one or more polymorphisms in plants of the population is measured, thereby permitting identification of whether or not the one or more polymorphisms are associated with such variation.

In another aspect, methods of making a plant line is provided. The methods include determining whether one or more genetic polymorphisms in a population of plants is associated with the locus for a polypeptide selected from the group consisting of the polypeptides depicted in FIGS. 1-16, functional homologs thereof, and polypeptide sequences in the sequence listing, identifying one or more plants in the population in which the presence of at least one allele at the one or more polymorphisms is associated with variation in salt tolerance or oxidative stress tolerance, crossing each of the one or more identified plants with itself or a different plant to produce seed, crossing at least one progeny plant grown from said seed with itself or a different plant, and repeating the crossing steps for an additional 0-5 generations to make the plant line. The at least one allele will be present in the plant line. The method of making a plant line may be applied, for example, to a population of switchgrass plants.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. The word “comprising” in the claims may be replaced by “consisting essentially of” or by “consisting of,” according to standard practice in patent law.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

FIG. 1 is an alignment of amino acid sequences of examples of homologues of Ceres clone no. 210210 (SEQ ID NO: 2), including Public GI ID no. 56202240 (SEQ ID NO: 7) and Ceres ANNOT ID no. 6044037 (SEQ ID NO: 9). In all the alignment Figures shown herein, a dash in an aligned sequence represents a gap, i.e., a lack of an amino acid at that position. Identical amino acids or conserved amino acid substitutions among aligned sequences are identified by boxes. FIG. 1 and the other alignment Figures provided herein were generated using the program MUSCLE version 3.52.

FIG. 2 is an alignment of amino acid sequences of examples of homologues of Ceres CLONE ID no. 22538 (SEQ ID NO: 11), including Ceres CLONE ID no. 1918442 (SEQ ID NO:13), Ceres ANNOT ID no. 1469916 (SEQ ID NO: 15), Ceres CLONE ID no. 872124 (SEQ ID NO: 17), Ceres CLONE ID no. 1076554 (SEQ ID NO: 19), Ceres CLONE ID no. 1725271 (SEQ ID NO: 23), and Public GI ID no. 115461767 (SEQ ID NO: 30).

FIG. 3 is an alignment of amino acid sequences of examples of homologues of Ceres CLONE ID no. 560692 (SEQ ID NO: 102), including Public GI ID no. 40807635 (SEQ ID NO: 103), Ceres CLONE ID no. 1798511 (SEQ ID NO: 106), Ceres ANNOT ID no. 1453285 (SEQ ID NO: 110), Ceres CLONE ID no. 31680 (SEQ ID NO: 112), Ceres CLONE ID no. 1117567 (SEQ ID NO: 114), Public GI ID no. 89257437 (SEQ ID NO: 115), Public GI ID no. 92874200 (SEQ ID NO: 116), Public GI ID no. 122230834 (SEQ ID NO: 120), Ceres CLONE ID no. 888852 (SEQ ID NO: 122), Ceres CLONE ID no. 1389074 (SEQ ID NO: 124), Public GI ID no. 11596186 (SEQ ID NO: 125), Ceres CLONE ID no. 1771297 (SEQ ID NO: 127), Public GI ID no. 109238661 (SEQ ID NO: 129), and Ceres ANNOT ID no. 6067853 (SEQ ID NO:134).

FIG. 4 is an alignment of amino acid sequences of examples of homologues of Ceres CLONE ID no. 547495 (SEQ ID NO: 32), including Public GI ID no. 400991 (SEQ ID NO: 33), Ceres CLONE ID no. 1808896 (SEQ ID NO: 35), Ceres ANNOT ID no. 1528012 (SEQ ID NO: 42), Ceres CLONE ID no. 15384 (SEQ ID NO: 48), Public GI ID no. 92885255 (SEQ ID NO: 63), Public GI ID no. 498902 (SEQ ID NO: 64), Public GI ID no. 8131701 (SEQ ID NO: 68), Public GI ID no. 730547 (SEQ ID NO: 69), Ceres CLONE ID no. 567641 (SEQ ID NO: 71), Ceres CLONE ID no. 1450693 (SEQ ID NO:79), Ceres CLONE ID no. 1731902 (SEQ ID NO: 89), Ceres CLONE ID no. 1760193 (SEQ ID NO: 91), Public GI ID no. 115445581 (SEQ ID NO: 98), and Public GI ID no. 50843988 (SEQ ID NO: 100).

FIG. 5 is an alignment of amino acid sequences of examples of homologues of Ceres LOCUS ID no. At3g45100 (SEQ ID NO: 138), including Ceres ANNOT ID no. 1476065 (SEQ ID NO: 142), Ceres CLONE ID no. 243208 (SEQ ID NO: 146), and Public GI ID no. 22296298 (SEQ ID NO: 147).

FIG. 6 is an alignment of amino acid sequences of examples of homologues of Ceres LOCUS ID no. At4g00150 SEQ ID NO: 149), including Ceres ANNOT ID no. 1539565 SEQ ID NO: 151), Public GI ID no. 22087131 SEQ ID NO: 155), Ceres CLONE ID no. 788036 SEQ ID NO: 157), Ceres CLONE ID no. 282885 SEQ ID NO: 159), Ceres CLONE ID no. 1806177 SEQ ID NO: 161), Public GI ID no. 125553722 SEQ ID NO: 163), Public GI ID no. 115459828 SEQ ID NO: 164), and Ceres ANNOT ID no. 6017571 SEQ ID NO: 167).

FIG. 7 is an alignment of amino acid sequences of examples of homologues of Ceres SEEDLINE ID no. ME06336 (SEQ ID NO: 441), including Ceres CLONE ID no. 1868840 (SEQ ID NO: 597), Public GI ID no. 115458894 (SEQ ID NO: 598), Ceres ANNOT ID no. 1472214 (SEQ ID NO: 600), Public GI ID no. 119224822 (SEQ ID NO: 601), Public GI ID no. 71834076 (SEQ ID NO: 606), Public GI ID no. 116876026 (SEQ ID NO: 609), Public GI ID no. 116248050 (SEQ ID NO: 610), and Public GI ID no. 45260636 (SEQ ID NO: 626).

FIG. 8 is an alignment of amino acid sequences of examples of homologues of Ceres SEEDLINE ID no. ME01395 (SEQ ID NO: 172), including Ceres CLONE ID no. 151087 (SEQ ID NO: 174), Public GI ID no. 10177809 (SEQ ID NO: 175), Ceres ANNOT ID no. 1504145 (SEQ ID NO: 178), Public GI ID no. 15241572 (SEQ ID NO: 179), Public G1 ID no. 26453064 (SEQ ID NO: 180), Ceres ANNOT ID no. 1451079 (SEQ ID NO: 182), Ceres CLONE ID no. 1786527 (SEQ ID NO: 184), Ceres CLONE ID no. 1005083 (SEQ ID NO: 186), Public GI ID no. 115447467 (SEQ ID NO: 187), Public GI ID no. 125582985 (SEQ ID NO: 188), Public GI ID no. 8346564 (SEQ ID NO: 189), Ceres CLONE ID no. 667071 (SEQ ID NO: 191), Public GI ID no. 125540418 (SEQ ID NO: 192), Ceres ANNOT ID no. 1451080 (SEQ ID NO: 194), Ceres ANNOT ID no. 1504146 (SEQ ID NO: 196), and Ceres ANNOT ID no. 6113886 (SEQ ID NO: 198).

FIG. 9 is an alignment of amino acid sequences of examples of homologues of Ceres SEEDLINE ID no. ME08564 (SEQ ID NO: 764), including Ceres CLONE ID no. 1928887 (SEQ ID NO: 836), Ceres ANNOT ID no. 1455474 (SEQ ID NO: 840), Public G1 ID no. 849072 (SEQ ID NO: 841), Public GI ID no. 22326727 (SEQ ID NO: 847), Public G1 ID no. 115488682 (SEQ ID NO: 850), Public GI ID no. 562188 (SEQ ID NO: 856), Public G1 ID no. 1245816 (SEQ ID NO: 857), and Ceres CLONE ID no. 375085 (SEQ ID NO: 860).

Ceres SEEDLINE ID no. ME24091 (SEQ ID NO: 922), Ceres CLONE ID no. 1942871 (SEQ ID NO: 924), Ceres ANNOT ID no. 1528800 (SEQ ID NO: 944), Ceres CLONE ID no. 467508 (SEQ ID NO: 981), Public GI ID no. 92868235 (SEQ ID NO: 982), Ceres CLONE ID no. 228069 (SEQ ID NO: 987), and Public GI ID no. 54306075 (SEQ ID NO: 995).

FIG. 11 is an alignment of amino acid sequences of examples of homologues of Ceres SEEDLINE ID no. ME03047 (SEQ ID NO: 316), including Ceres CLONE ID no. 1064300 (SEQ ID NO: 331), Ceres CLONE ID no. 613904 (SEQ ID NO:344), Ceres CLONE ID no. 1932709 (SEQ ID NO: 353), Ceres ANNOT ID no. 1494788 (SEQ ID NO: 355), Public GI ID no. 115489526 (SEQ ID NO: 356), Ceres CLONE ID no. 579413 (SEQ ID NO: 359), and Ceres CLONE ID no. 1874905 (SEQ ID NO: 361).

FIG. 12 is an alignment of amino acid sequences of examples of homologues of Ceres SEEDLINE ID no. ME02441 (SEQ ID NO: 200), including Ceres ANNOT ID no. 1491256 (SEQ ID NO: 204), Ceres CLONE ID no. 1053866 (SEQ ID NO: 217), Ceres CLONE ID no. 1526732 (SEQ ID NO: 219), Ceres CLONE ID no. 2026154 (SEQ ID NO: 230), and Public GI ID no. 115455695 (SEQ ID NO: 233).

FIG. 13 is an alignment of amino acid sequences of examples of homologues of Ceres SEEDLINE ID no. ME08052 (SEQ ID NO: 697), including Public GI ID no. 115447377 (SEQ ID NO: 706), Ceres CLONE ID no. 696883 (SEQ ID NO: 709), Ceres ANNOT ID no. 1462901 (SEQ ID NO: 711), Ceres CLONE ID no. 1794111 (SEQ ID NO:714), and Ceres CLONE ID no. 1240524 (SEQ ID NO: 718).

FIG. 14 is an alignment of amino acid sequences of examples of homologues of Ceres SEEDLINE ID no. ME02927 (SEQ ID NO: 239), including Public G1 ID no. 62751057 (SEQ ID NO: 273), Ceres CLONE ID no. 1839135 (SEQ ID NO: 275), Ceres CLONE ID no. 1371813 (SEQ ID NO: 279), Public GI ID no. 40809629 (SEQ ID NO: 291), Ceres CLONE ID no. 1720386 (SEQ ID NO: 297), and Ceres CLONE ID no. 391080 (SEQ ID NO: 299).

FIG. 15 is an alignment of amino acid sequences of examples of homologues of Ceres SEEDLINE ID no. ME11932 (SEQ ID NO: 892), including Ceres ANNOT ID no. 1471603 (SEQ ID NO: 894), Ceres CLONE ID no. 949027 (SEQ ID NO: 903), Public G1 ID no. 92877259 (SEQ ID NO: 906), Public GI ID no. 61611694 (SEQ ID NO: 907), Ceres CLONE ID no. 779610 (SEQ ID NO: 909), Ceres CLONE ID no. 226231 (SEQ ID NO: 911), and Public GI ID no. 125592880 (SEQ ID NO: 916).

FIG. 16 is an alignment of amino acid sequences of examples of homologues of Ceres SEEDLINE ID no. ME24091 (SEQ ID NO: 922), Ceres CLONE ID no. 1942871 (SEQ ID NO: 924), Ceres ANNOT ID no. 1528800 (SEQ ID NO: 944), Ceres CLONE ID no. 467508 (SEQ ID NO: 981), Public GI ID no. 92868235 (SEQ ID NO: 982), Ceres CLONE ID no. 228069 (SEQ ID NO: 987), and Public GI ID no. 54306075 (SEQ ID NO: 995).

### DETAILED DESCRIPTION

The invention features methods and materials related to modulating salinity and/or oxidative stress tolerance levels in plants and/or plant tissues. In some embodiments, the plants may also have increased biomass and/or yield. The methods can include transforming a plant cell with a nucleic acid encoding a salinity and/or oxidative stress tolerance-modulating polypeptide, wherein expression of the polypeptide results in a modulated level of salinity and/or oxidative stress tolerance. Plant cells produced using such methods can be grown to produce plants having an increased salinity, oxidative stress tolerance, and/or biomass, in comparison to wild type plants grown under the same conditions. Such plants, and the seeds of such plants, may be used to produce, for example, yield and/or biomass utilized for biofuel production, such as, but not limited to, ethanol, butanol, and thermochemical conversion.

### I. DEFINITIONS

“Amino acid” refers to one of the twenty biologically occurring amino acids and to synthetic amino acids, including D/L optical isomers.

“Cell type-preferential promoter” or “tissue-preferential promoter” refers to a promoter that drives expression preferentially in a target cell type or tissue, respectively, but may also lead to some transcription in other cell types or tissues as well.

“Control plant” refers to a plant that does not contain the exogenous nucleic acid present in a transgenic plant of interest, but otherwise has the same or similar genetic background as such a transgenic plant. A suitable control plant can be a non-transgenic wild type plant, a non-transgenic segregant from a transformation experiment, or a transgenic plant that contains an exogenous nucleic acid other than the exogenous nucleic acid of interest.

“Domains” are groups of substantially contiguous amino acids in a polypeptide that can be used to characterize protein families and/or parts of proteins. Such domains have a “fingerprint” or “signature” that can comprise conserved primary sequence, secondary structure, and/or three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or to 65 amino acids, or 35 to 55 amino acids, or 45 to 60 amino acids, or 200 to 300 amino acids, or 300 to 400 amino acids.

“Down-regulation” refers to regulation that decreases production of expression products (mRNA, polypeptide, or both) relative to basal or native states.

“Exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

“Expression” refers to the process of converting genetic information of a polynucleotide into RNA through transcription, which is catalyzed by an enzyme, RNA polymerase, and into protein, through translation of mRNA on ribosomes.

“Heterologous polypeptide” as used herein refers to a polypeptide that is not a naturally occurring polypeptide in a plant cell, e.g., a transgenic Panicum virgatum plant transformed with and expressing the coding sequence for a nitrogen transporter polypeptide from a Zea mays plant.

“Isolated nucleic acid” as used herein includes a naturally-occurring nucleic acid, provided that one or both of the sequences immediately flanking that nucleic acid in its naturally-occurring genome is removed or absent. Thus, an isolated nucleic acid includes, without limitation, a nucleic acid that exists as a purified molecule or a nucleic acid molecule that is incorporated into a vector or a virus. A nucleic acid existing among hundreds to millions of other nucleic acids within, for example, cDNA libraries, genomic libraries, or gel slices containing a genomic DNA restriction digest, is not to be considered an isolated nucleic acid.

“Modulation” of the level of salt tolerance and/or oxidative stress tolerance refers to the change in the level of the salt tolerance and/or oxidative stress tolerance that is observed as a result of expression of, or transcription from, an exogenous nucleic acid in a plant cell. The change in level is measured relative to the corresponding level in control plants.

“Nucleic acid” and “polynucleotide” are used interchangeably herein, and refer to both RNA and DNA, including cDNA, genomic DNA, synthetic DNA, and DNA or RNA containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, nucleic acid probes and nucleic acid primers. A polynucleotide may contain unconventional or modified nucleotides.

“Operably linked” refers to the positioning of a regulatory region and a sequence to be transcribed in a nucleic acid so that the regulatory region is effective for regulating transcription or translation of the sequence. For example, to operably link a coding sequence and a regulatory region, the translation initiation site of the translational reading frame of the coding sequence is typically positioned between one and about fifty nucleotides downstream of the regulatory region. A regulatory region can, however, be positioned as much as about 5,000 nucleotides upstream of the translation initiation site, or about 2,000 nucleotides upstream of the transcription start site.

“Polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. Full-length polypeptides, truncated polypeptides, point mutants, insertion mutants, splice variants, chimeric proteins, and fragments thereof are encompassed by this definition.

“Progeny” includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). For example, a suitable enhancer is a cis-regulatory element (-212 to -154) from the upstream region of the octopine synthase (ocs) gene. Fromm et al., The Plant Cell, 1:977-984 (1989).

“Up-regulation” refers to regulation that increases the level of an expression product (mRNA, polypeptide, or both) relative to basal or native states.

“Vector” refers to a replicon, such as a plasmid, phage, or cosmid, into which another DNA segment may be inserted so as to bring about the replication of the inserted segment. Generally, a vector is capable of replication when associated with the proper control elements. The term “vector” includes cloning and expression vectors, as well as viral vectors and integrating vectors. An “expression vector” is a vector that includes a regulatory region.

“Oxidative stress” can be defined as the set of environmental conditions under which a plant will begin to suffer the effects of elevated ROS/ROI/AOS concentration, such as decreases in enzymatic activity, DNA breakage, DNA-protein crosslinking, necrosis and stunted growth. For these reasons, plants experiencing oxidative stress typically exhibit a significant reduction in biomass and/or yield. Plant species vary in their capacity to tolerate ROS/ROI/AOS.

Elevated oxidative stress may be caused by natural, geological processes and by human activities, such as pollution. Since plant species vary in their capacity to tolerate oxidative stress, the precise environmental conditions that cause stress cannot be generalized. However, under oxidative stress conditions, oxidative stress tolerant plants produce higher biomass, yield and survivorship than plants that are not oxidative stress tolerant. Differences in physical appearance, recovery and yield can be quantified.

Photosynthetic efficiency: photosynthetic efficiency, or electron transport via photosystem II, is estimated by the relationship between Fm, the maximum fluorescence signal and the variable fluorescence, Fv. A reduction in the optimum quantum yield (Fv/Fm) indicates stress and can be used to monitor the performance of transgenic plants compared to non-transgenic plants under salt or oxidative stress conditions.

Salicylic Acid Growth Index (SAGI): Photosynthetic efficiency×seedling area.

Salt growth index (SGI): Photosynthetic efficiency×seedling area (under salinity stress condition).

Salinity: Plant species vary in their capacity to tolerate salinity “Salinity” can be defined as the set of environmental conditions under which a plant will begin to suffer the effects of elevated salt concentration, such as ion imbalance, decreased stomatal conductance, decreased photosynthesis, decreased growth rate, increased cell death, loss of turgor (wilting), or ovule abortion. For these reasons, plants experiencing salinity stress typically exhibit a significant reduction in biomass and/or yield.

Elevated salinity may be caused by natural, geological processes and by human activities, such as pollution. Since plant species vary in their capacity to tolerate salinity, the precise environmental conditions that cause stress cannot be generalized. However, under saline conditions, salinity tolerant plants produce higher biomass, yield and survivorship than plants that are not saline tolerant. Differences in physical appearance, recovery and yield can be quantified.

Elevated salinity may be caused by natural, geological processes and by human activities, such as irrigation. Since plant species vary in their capacity to tolerate water deficit, the precise environmental salt conditions that cause stress cannot be generalized. However, under saline conditions, salt tolerant plants produce higher biomass, yield and survivorship than plants that are not salt tolerant. Differences in physical appearance, recovery and yield can be quantified and statistically analyzed using well known measurement and analysis methods.

### II. POLYPEPTIDES

Polypeptides described herein include salinity and/or oxidative stress tolerance-modulating polypeptides. Salinity and/or oxidative stress tolerance-modulating polypeptides can be effective to modulate salinity and/or oxidative stress tolerance levels when expressed in a plant or plant cell. Such polypeptides typically contain at least one domain indicative of salinity and/or oxidative stress tolerance-modulating polypeptides, as described in more detail herein. Salinity and/or oxidative stress tolerance-modulating polypeptides typically have an HMM bit score that is greater than 15, as described in more detail herein. In some embodiments, salinity and/or oxidative stress tolerance-modulating polypeptides have 80% or greater identity to SEQ ID NOs: 2, 11, 102, 32, 138, 149, 441, 172, 764, 389, 316, 200, 697, 239, 892, 922, or 693 as described in more detail herein.

A. Domains Indicative of Salinity and/or Oxidative Stress Tolerance-Modulating Polypeptides

A salinity and/or oxidative stress tolerance-modulating polypeptide can contain a XYPPX repeat domain, which is predicted to be characteristic of a salinity and/or oxidative stress tolerance-modulating polypeptide. For example, SEQ ID NO: 11 sets forth the amino acid sequence of an Arabidopsis clone, identified herein as Ceres CLONE ID no. 22538, that is predicted to encode a polypeptide containing XYPPX repeat domains from residues 60 to 64, 69 to 73, 78 to 82, 87 to 91, and 15 to 19. Examples of other XYPPX domains are shown in the sequence listing. This repeat is found in a wide variety of proteins and generally consists of the motif XYPPX where X can be any amino acid. The family includes annexin VII ANX7_DICDI, the carboxy tail of certain rhodopsins OPSD_LOLSU. This family also includes plaque matrix proteins; however this motif is embedded in a ten residue repeat in FP1_MYTED.

A salinity and/or oxidative stress tolerance-modulating polypeptide can contain a Cystatin domain, which is predicted to be characteristic of a salinity and/or oxidative stress tolerance-modulating polypeptide. For example, SEQ ID NO: 102 sets forth the amino acid sequence of an Glycine max clone, identified herein as Ceres CLONE ID no. 560692, that is predicted to encode a polypeptide containing a Cystatin domain from residues 27 to 87. Examples of other Cystatin domains are shown in the sequence listing. Peptide proteinase inhibitors contain Cystatin domains and can be found as single domain proteins or as single or multiple domains within proteins; these are referred to as either simple or compound inhibitors, respectively. In many cases they are synthesised as part of a larger precursor protein, either as a prepropeptide or as an N-terminal domain associated with an inactive peptidase or zymogen. This domain prevents access of the substrate to the active site. Removal of the N-terminal inhibitor domain either by interaction with a second peptidase or by autocatalytic cleavage activates the zymogen. Other inhibitors interact direct with proteinases using a simple noncovalent lock and key mechanism; while yet others use a conformational change-based trapping mechanism that depends on their structural and thermodynamic properties.

A salinity and/or oxidative stress tolerance-modulating polypeptide can contain an Kyprides, Ouzounis, Woese (KOW) motif domain, which is predicted to be characteristic of a salinity and/or oxidative stress tolerance-modulating polypeptide. For example, SEQ ID NO: 32 sets forth the amino acid sequence of an Glycine max clone, identified herein as Ceres CLONE ID no. 547495, that is predicted to encode a polypeptide containing a KOW motif domain from residues 7 to 40. Examples of other KOW domains are shown in the sequence listing. The KOW motif is found in a variety of ribosomal proteins and NusG. Ribosomes are the particles that catalyze mRNA-directed protein synthesis in all organisms. The codons of the mRNA are exposed on the ribosome to allow tRNA binding. This leads to the incorporation of amino acids into the growing polypeptide chain in accordance with the genetic information. Incoming amino acid monomers enter the ribosomal A site in the form of aminoacyl-tRNAs complexed with elongation factor Tu (EF-Tu) and GTP. The growing polypeptide chain, situated in the P site as peptidyl-tRNA, is then transferred to aminoacyl-tRNA and the new peptidyl-tRNA, extended by one residue, is translocated to the P site with the aid the elongation factor G (EF-G) and GTP as the deacylated tRNA is released from the ribosome through one or more exit sites. About ⅔ of the mass of the ribosome consists of RNA and ⅓ of protein. The proteins are named in accordance with the subunit of the ribosome which they belong to—the small (S1 to S31) and the large (L1 to L44). Usually they decorate the rRNA cores of the subunits.

A salinity and/or oxidative stress tolerance-modulating polypeptide that contains a KOW motif domain can also contain a Ribosomal L27e protein family domain, which is predicted to be characteristic of a salinity and/or oxidative stress tolerance-modulating polypeptide. For example, SEQ ID NO: 32 sets forth the amino acid sequence of an Glycine max clone, identified herein as Ceres CLONE ID no. 547495, that is predicted to encode a polypeptide containing a Ribosomal L27e protein family domain from residues 52 to 135. Examples of other Ribosomal L27e protein family domains are shown in the sequence listing. The N-terminal region of the eukaryotic ribosomal L27 has the KOW motif. C-terminal region is represented by the Ribosomal L27e protein family.

A salinity and/or oxidative stress tolerance-modulating polypeptide can contain a Glycosyl transferases group 1 domain, which is predicted to be characteristic of a salinity and/or oxidative stress tolerance-modulating polypeptide. For example, SEQ ID NO: 138 sets forth the amino acid sequence of an Arabidopsis clone, identified herein as Ceres LOCUS ID no. At3g45100, that is predicted to encode a polypeptide containing a Glycosyl transferases group 1 domain from residues 187 to 356. Examples of other Glycosyl transferases group 1 domains are shown in the sequence listing. The biosynthesis of disaccharides, oligosaccharides and polysaccharides involves the action of hundreds of different glycosyltransferases. These enzymes catalyse the transfer of sugar moieties from activated donor molecules to specific acceptor molecules, forming glycosidic bonds. A classification of glycosyltransferases using nucleotide diphospho-sugar, nucleotide monophospho-sugar and sugar phosphates and related proteins into distinct sequence based families has been described. The same three-dimensional fold is expected to occur within each of the families. Because 3-D structures are better conserved than sequences, several of the families defined on the basis of sequence similarities may have similar 3-D structures and therefore form ‘clans’. Proteins containingn this domain transfer UDP, ADP, GDP or CMP linked sugars to a variety of substrates, including glycogen, fructose-6-phosphate and lipopolysaccharides. The bacterial enzymes are involved in various biosynthetic processes that include exopolysaccharide biosynthesis, lipopolysaccharide core biosynthesis and the biosynthesis of the slime polysaccaride colanic acid.

A salinity and/or oxidative stress tolerance-modulating polypeptide can contain a Glycosyl transferases group 1 domain and additionally contain a PIGA (GPI anchor biosynthesis) domain, which is predicted to be characteristic of a salinity and/or oxidative stress tolerance-modulating polypeptide. For example, SEQ ID NO: 138 sets forth the amino acid sequence of an Arabidopsis clone, identified herein as Ceres LOCUS ID no. At3g45100, which is predicted to encode a polypeptide containing a PIGA domain from residues 46 to 135. Examples of other PIGA domains are shown in the sequence listing. This domain is found on phosphatidylinositol n-acetylglucosaminyltransferase proteins. These proteins are involved in GPI anchor biosynthesis and are associated with disease the paroxysmal nocturnal haemoglobinuria.

A salinity and/or oxidative stress tolerance-modulating polypeptide can contain a GRAS family transcription factor domain, which is predicted to be characteristic of a salinity and/or oxidative stress tolerance-modulating polypeptide. For example, SEQ ID NO: 149 sets forth the amino acid sequence of an Arabidopsis clone, identified herein as Ceres LOCUS ID no. At4g00150, which is predicted to encode a polypeptide containing a GRAS family transcription factor domain from residues 179 to 467. Examples of other GRAS family transcription factor domains are shown in the sequence listing. Sequence analysis of the products of the GRAS (GAI, RGA, SCR) gene family indicates that they share a variable N-terminus and a highly conserved C-terminus that contains five recognizable motifs. Proteins in the GRAS family are transcription factors that seem to be involved in development and other processes. Mutation of the SCARECROW (SCR) gene results in a radial pattern defect, loss of a ground tissue layer, in the root. The PAT1 protein is involved in phytochrome A signal transduction. GRAS proteins contain a conserved region of about 350 amino acids that can be divided in 5 motifs, found in the following order: leucine heptad repeat I, the VHIID motif, leucine heptad repeat II, the PFYRE motif and the SAW motif. Plant specific GRAS proteins have parallels in their motif structure to the animal Signal Transducers and Activators of Transcription (STAT) family of proteins which suggests also some parallels in their functions.

A salinity and/or oxidative stress tolerance-modulating polypeptide can contain a Cytochrome P450 domain, which is predicted to be characteristic of a salinity and/or oxidative stress tolerance-modulating polypeptide. For example, SEQ ID NO: 441 sets forth the amino acid sequence of an Arabidopsis clone, identified herein as Ceres SEEDLINE ID no. ME06336, which is predicted to encode a polypeptide containing a Cytochrome P450 domain from residues 228 to 466. Examples of other Cytochrome P450 domains are shown in the sequence listing. Cytochrome P450s are haem-thiolate proteins involved in the oxidative degradation of various compounds. They are particularly well known for their role in the degradation of environmental toxins and mutagens. The family of P450s is divided into numerous subfamilies, for example, the sequence of SEQ ID NO:144 falls within the CYP724B subfamily, and further within the third group of the subfamily CYP724B3. Sequence conservation is relatively low within the family—there are only 3 absolutely conserved residues—but their general topography and structural fold are highly conserved. The conserved core is composed of a coil termed the ‘meander’, a four-helix bundle, helices J and K, and two sets of beta-sheets. These constitute the haem-binding loop (with an absolutely conserved cysteine that serves as the 5th ligand for the haem iron), the proton-transfer groove and the absolutely conserved EXXR motif in helix K. While prokaryotic P450s are soluble proteins, most eukaryotic P450s are associated with microsomal membranes. Their general enzymatic function is to catalyse regiospecific and stereospecific oxidation of non-activated hydrocarbons at physiological temperatures.

A salinity and/or oxidative stress tolerance-modulating polypeptide can contain a Cyclin, N-terminal domain, which is predicted to be characteristic of a salinity and/or oxidative stress tolerance-modulating polypeptide. For example, SEQ ID NO: 764 sets forth the amino acid sequence of an Arabidopsis clone, identified herein as Ceres SEEDLINE ID no. ME08564, which is predicted to encode a polypeptide containing a Cyclin, N-terminal domain from residues 196 to 323. Examples of other Cyclin, N-terminal domains are shown in the sequence listing. Cyclins are eukaryotic proteins that play an active role in controlling nuclear cell division cycles, and regulate cyclin dependent kinases (CDKs). Cyclins, together with the p34 (cdc2) or cdk2 kinases, form the Maturation Promoting Factor (MPF). There are two main groups of cyclins, G1/S cyclins, which are essential for the control of the cell cycle at the G1/S (start) transition, and G2/M cyclins, which are essential for the control of the cell cycle at the G2/M (mitosis) transition. G2/M cyclins accumulate steadily during G2 and are abruptly destroyed as cells exit from mitosis (at the end of the M-phase). In most species, there are multiple forms of G1 and G2 cyclins. For example, in vertebrates, there are two G2 cyclins, A and B, and at least three G1 cyclins, C, D, and E.

A salinity and/or oxidative stress tolerance-modulating polypeptide can contain a Cyclin, N-terminal domain and additionally a Cyclin, C-terminal domain, which is predicted to be characteristic of a salinity and/or oxidative stress tolerance-modulating polypeptide. For example, SEQ ID NO: 764 sets forth the amino acid sequence of an Arabidopsis clone, identified herein as Ceres SEEDLINE ID no. ME08564, which is predicted to encode a polypeptide containing a Cyclin, C-terminal domain from residues 325 to 452. Examples of other Cyclin, C-terminal domains are shown in the sequence listing.

A salinity and/or oxidative stress tolerance-modulating polypeptide can contain a MATH domain, which is predicted to be characteristic of a salinity and/or oxidative stress tolerance-modulating polypeptide. For example, SEQ ID NO: 389 sets forth the amino acid sequence of an Arabidopsis clone, identified herein as Ceres SEEDLINE ID no. ME03280, which is predicted to encode a polypeptide containing a MATH domain from residues 245 to 370. Examples of other MATH domains are shown in the sequence listing. This motif has been called the Meprin and TRAF-Homology (MATH) domain. This domain is hugely expanded in the nematode C. elegans. Although apparently functionally unrelated, intracellular TRAFs and extracellular meprins share a conserved region of about 180 residues, the meprin and TRAF homology (MATH) domain. Meprins are mammalian tissue-specific metalloendopeptidases of the astacin family implicated in developmental, normal and pathological processes by hydrolyzing a variety of proteins. Various growth factors, cytokines, and extracellular matrix proteins are substrates for meprins. TRAF proteins were first isolated by their ability to interact with TNF receptors. They promote cell survival by the activation of downstream protein kinases and, finally, transcription factors of the NF-kB and AP-1 family. The TRAF proteins are composed of 3 structural domains: a RING finger in the N-terminal part of the protein, one to seven TRAF zinc in the middle and the MATH domain in the C-terminal part. The MATH domain is necessary and sufficient for self-association and receptor interaction.

A salinity and/or oxidative stress tolerance-modulating polypeptide can contain a LSD1 zinc finger domain, which is predicted to be characteristic of a salinity and/or oxidative stress tolerance-modulating polypeptide. For example, SEQ ID NO: 316 sets forth the amino acid sequence of an Arabidopsis clone, identified herein as Ceres SEEDLINE ID no. ME03047, which is predicted to encode a polypeptide containing a LSD1 zinc finger domain from residues 95 to 119. Examples of other LSD1 zinc finger domains are shown in the sequence listing. Also, for example, SEQ ID NO: 239 sets forth the amino acid sequence of an Arabidopsis clone, identified herein as Ceres SEEDLINE ID no. ME02927, which is predicted to encode a polypeptide containing a LSD1 zinc finger domain from residues 114 to 138. This domain family consists of several plant specific LSD1 zinc finger domains. Arabidopsis lsd1 mutants are hyper-responsive to cell death initiators and fail to limit the extent of cell death. Superoxide is a necessary and sufficient signal for cell death propagation. LSD1 monitors a superoxide-dependent signal and negatively regulates a plant cell death pathway. LSD1 protein contains three zinc finger domains, defined by CxxCxRxxLMYxxGASxVxCxxC. It has been suggested that LSD1 defines a zinc finger protein subclass and that LSD1 regulates transcription, via either repression of a pro-death pathway or activation of an anti-death pathway, in response to signals emanating from cells undergoing pathogen-induced hypersensitive cell death.

A salinity and/or oxidative stress tolerance-modulating polypeptide can contain a Glycolipid transfer protein (GLTP) domain, which is predicted to be characteristic of a salinity and/or oxidative stress tolerance-modulating polypeptide. For example, SEQ ID NO: 697 sets forth the amino acid sequence of an Arabidopsis clone, identified herein as Ceres SEEDLINE ID no. ME08052, which is predicted to encode a polypeptide containing a Glycolipid transfer protein (GLTP) domain from residues 10 to 198. Examples of other GLTP domains are shown in the sequence listing. GLTP is a cytosolic protein that catalyses the intermembrane transfer of glycolipids.

A salinity and/or oxidative stress tolerance-modulating polypeptide can contain a IQ calmodulin-binding motif domain, which is predicted to be characteristic of a salinity and/or oxidative stress tolerance-modulating polypeptide. For example, SEQ ID NO: 922 sets forth the amino acid sequence of an Arabidopsis clone, identified herein as Ceres SEEDLINE ID no. ME24091, that is predicted to encode a polypeptide containing an IQ calmodulin-binding motif domain from residues 115 to 135. Examples of other IQ calmodulin-binding motif domains are shown in the sequence listing. Calmodulin (CaM) is recognized as a major calcium sensor and orchestrator of regulatory events through its interaction with a diverse group of cellular proteins. Three classes of recognition motifs exist for many of the known CaM binding proteins; the IQ motif as a consensus for Ca2⁺-independent binding and two related motifs for Ca2⁺-dependent binding, termed 18-14 and 1-5-10 based on the position of conserved hydrophobic residues.

In some embodiments, a salinity and/or oxidative stress tolerance-modulating polypeptide is truncated at the amino- or carboxy-terminal end of a naturally occurring polypeptide. A truncated polypeptide may retain certain domains of the naturally occurring polypeptide while lacking others. Thus, length variants that are up to 5 amino acids shorter or longer typically exhibit the salinity and/or oxidative stress tolerance-modulating activity of a truncated polypeptide. In some embodiments, a truncated polypeptide is a dominant negative polypeptide. Expression in a plant of such a truncated polypeptide confers a difference in the level of salinity and/or oxidative stress tolerance in a plant and/or plant tissue as compared to the corresponding level a control plant and/or tissue thereof that does not comprise the truncation.

B. Functional Homologs Identified by Reciprocal BLAST

In some embodiments, one or more functional homologs of a reference salinity and/or oxidative stress tolerance-modulating polypeptide defined by one or more of the pfam descriptions indicated above are suitable for use as salinity and/or oxidative stress tolerance-modulating polypeptides. A functional homolog is a polypeptide that has sequence similarity to a reference polypeptide, and that carries out one or more of the biochemical or physiological function(s) of the reference polypeptide. A functional homolog and the reference polypeptide may be natural occurring polypeptides, and the sequence similarity may be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, may themselves be functional homologs. Functional homologs can also be created via site-directed mutagenesis of the coding sequence for a salinity and/or oxidative stress tolerance-modulating polypeptide, or by combining domains from the coding sequences for different naturally-occurring salinity and/or oxidative stress tolerance-modulating polypeptides (“domain swapping”). The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of salinity and/or oxidative stress tolerance-modulating polypeptides. Sequence analysis can involve BLAST, Reciprocal BLAST, or PSI-BLAST analysis of nonredundant databases using a salinity and/or oxidative stress tolerance-modulating polypeptide amino acid sequence as the reference sequence. Amino acid sequence is, in some instances, deduced from the nucleotide sequence. Those polypeptides in the database that have greater than 20% sequence identity are candidates for further evaluation for suitability as a salinity and/or oxidative stress tolerance-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in salinity and/or oxidative stress tolerance-modulating polypeptides, e.g., conserved functional domains.

Conserved regions can be identified by locating a region within the primary amino acid sequence of a salinity and/or oxidative stress tolerance-modulating polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species is adequate.

Typically, polypeptides that exhibit at least about 20% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

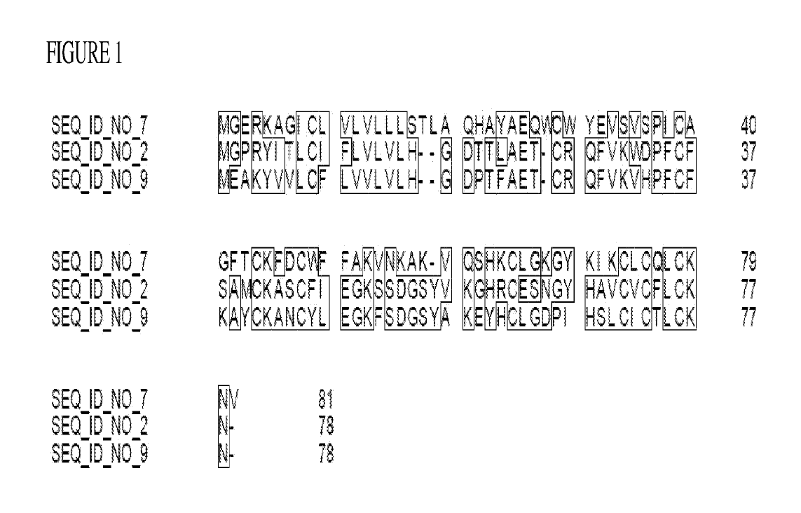

Examples of amino acid sequences of functional homologs of the polypeptide set forth in SEQ ID NO: 2 are provided in FIG. 1 and in the Sequence Listing. Such functional homologs include SEQ ID NO: 4, 6, 7, and 9. In some cases, a functional homolog of SEQ ID NO: 2 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 2.

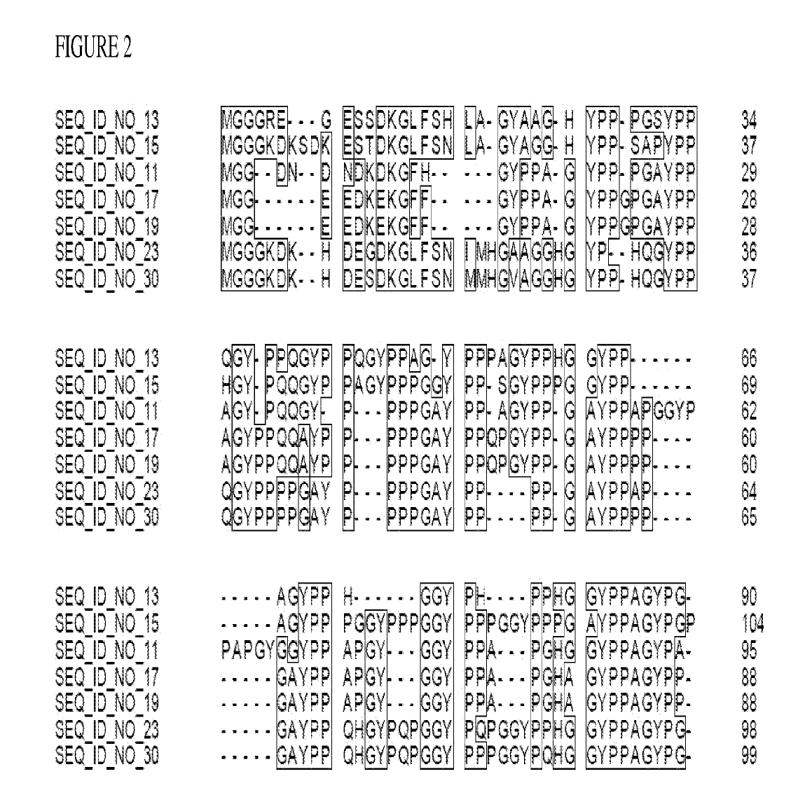

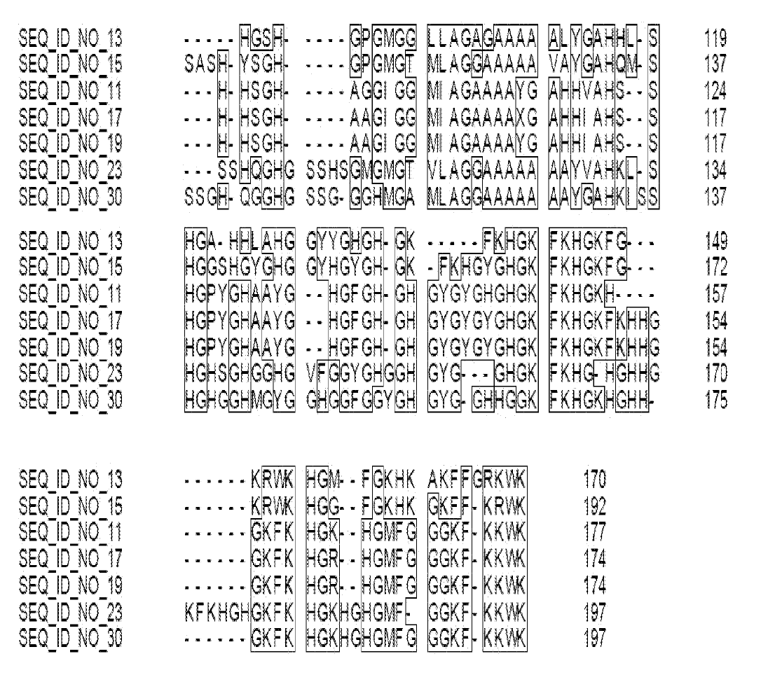

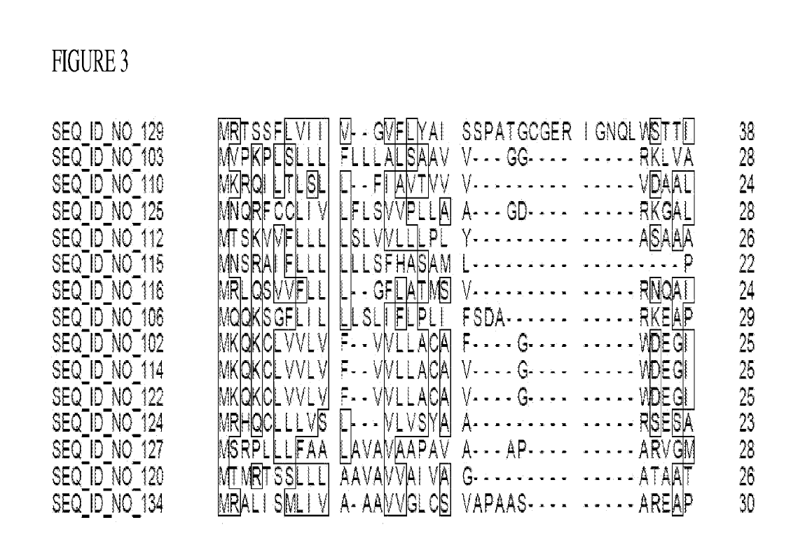

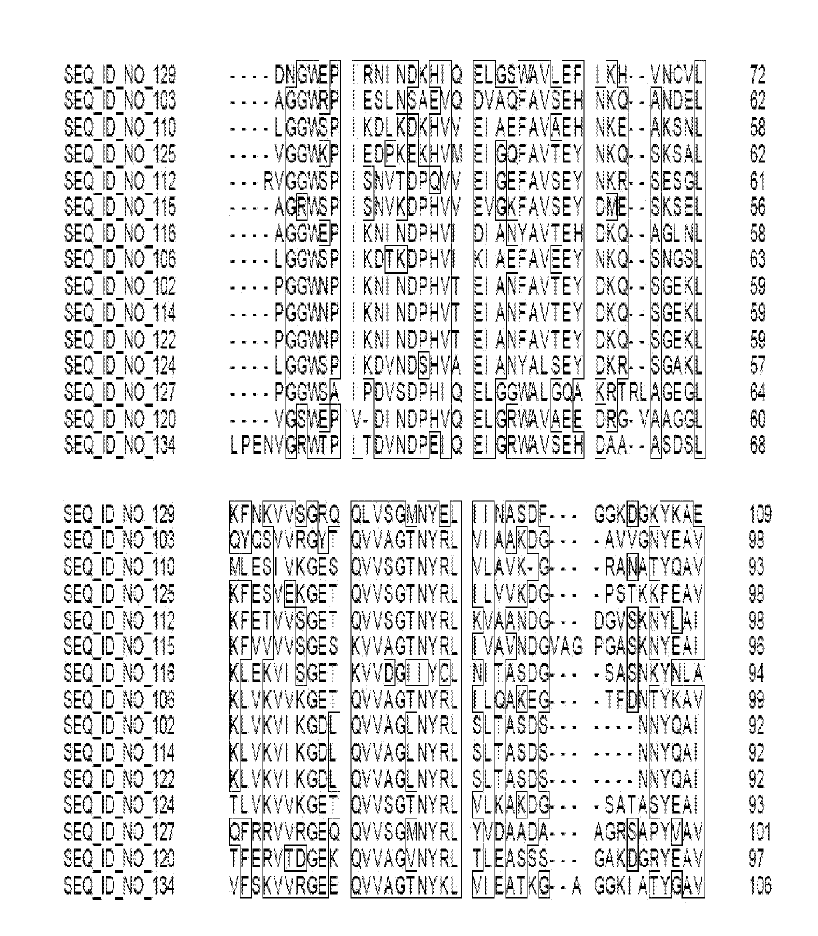

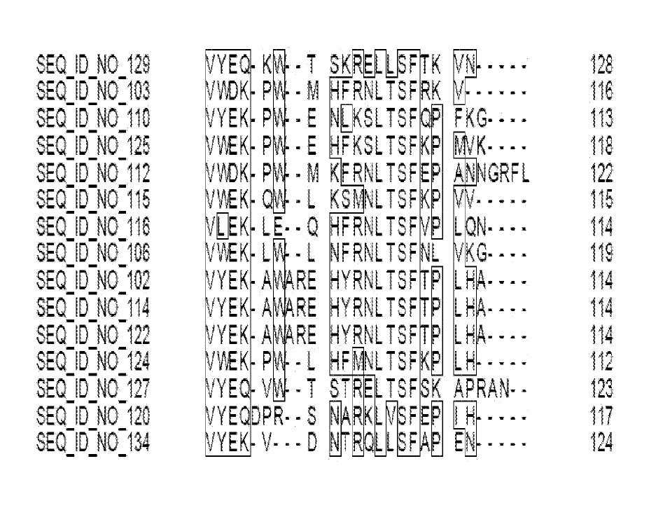

Examples of amino acid sequences of functional homologs of the polypeptide set forth in SEQ ID NO: 11 are provided in FIG. 2 and in the Sequence Listing. Such functional homologs include SEQ ID NO: 13, 15, 17, 19, 21, 23, 25, 27, 29, and 30. In some cases, a functional homolog of SEQ ID NO: 11 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 11.

Examples of amino acid sequences of functional homologs of the polypeptide set forth in SEQ ID NO: 102 are provided in FIG. 3 and in the Sequence Listing. Such functional homologs include (SEQ ID NO: 103, 104, 106, 108, 110, 112, 114, 115, 116, 117, 118, 119, 120, 122, 124, 125, 127, 128, 129, 130, 132, 134, and 136. In some cases, a functional homolog of SEQ ID NO: 102 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 102.

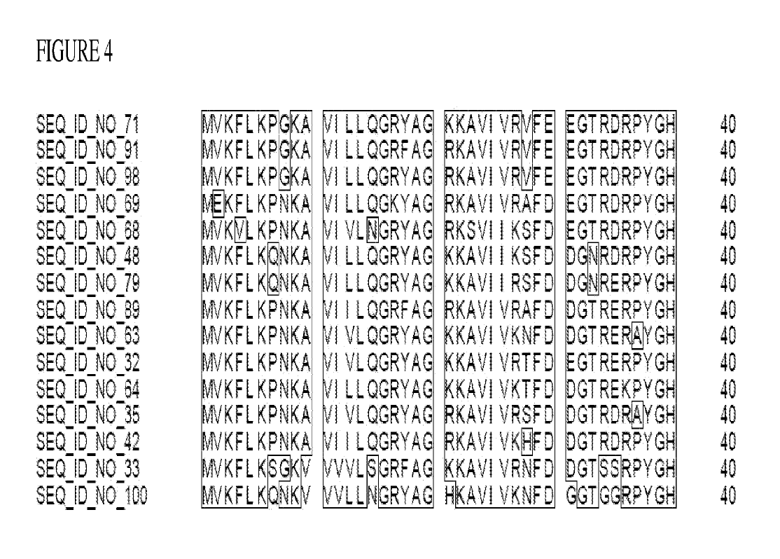

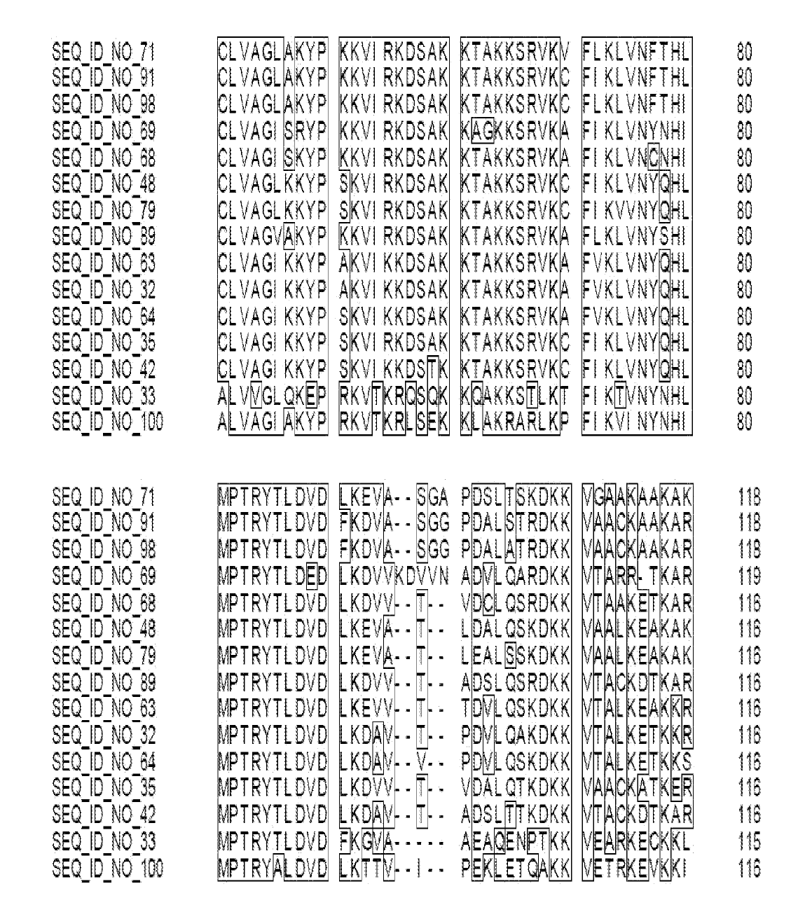

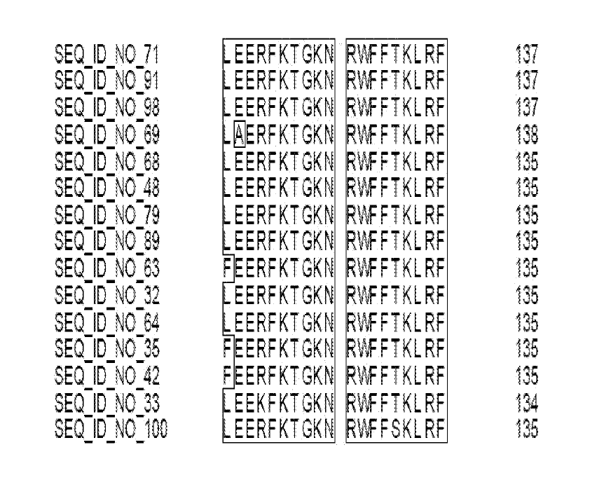

Examples of amino acid sequences of functional homologs of the polypeptide set forth in SEQ ID NO: 32 are provided in FIG. 4 and in the Sequence Listing. Such functional homologs include SEQ ID NO: 33, 35, 37, 38, 40, 42, 44, 46, 48, 49, 51, 53, 54, 56, 58, 60, 62, 63, 64, 65, 66, 67, 68, 69, 71, 73, 75, 77, 79, 81, 83, 85, 87, 89, 91, 93, 95, 97, 98, 99, and 100. In some cases, a functional homolog of SEQ ID NO: 32 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 32.

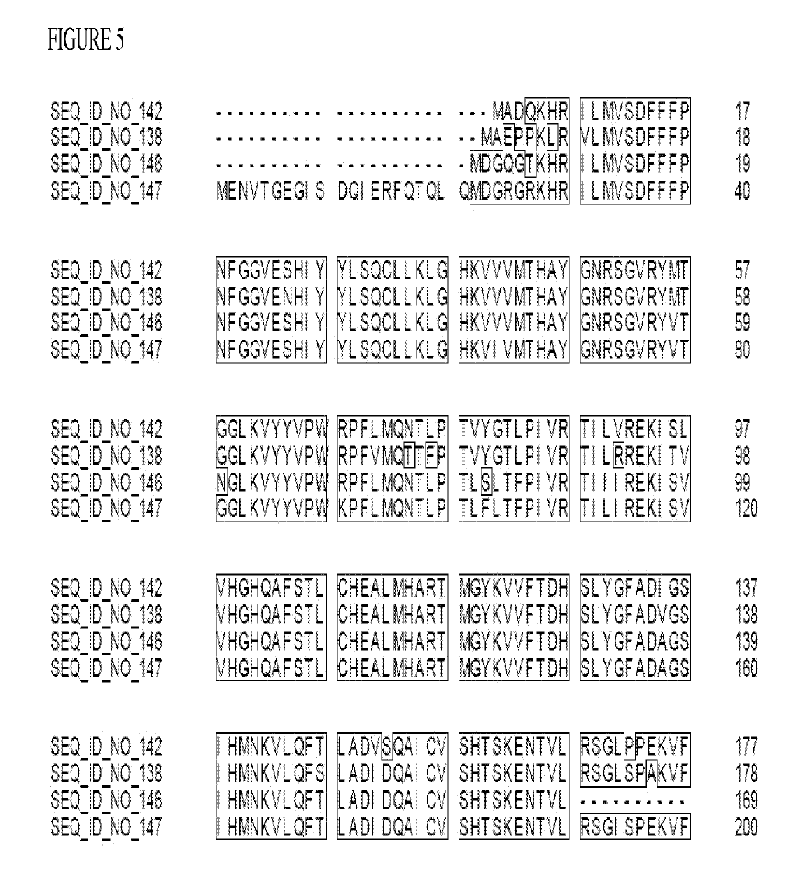

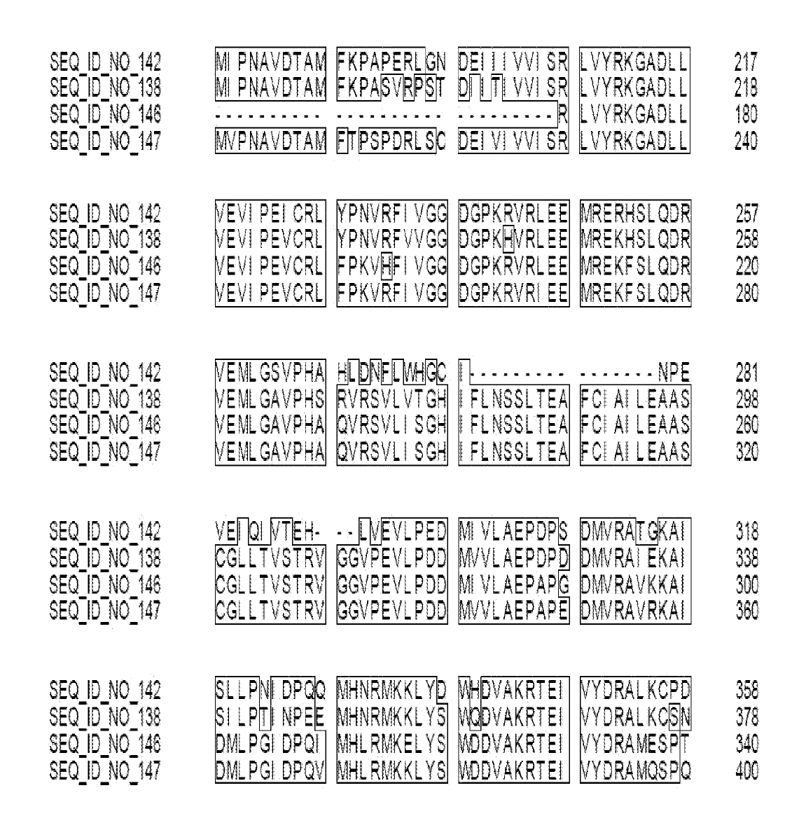

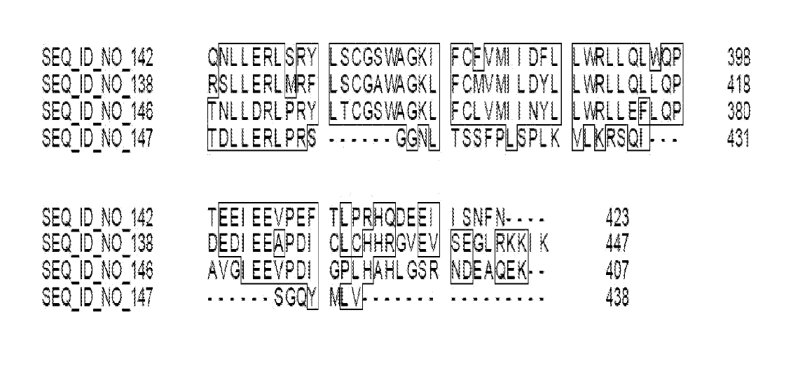

Examples of amino acid sequences of functional homologs of the polypeptide set forth in SEQ ID NO: 138 are provided in FIG. 5 and in the Sequence Listing. Such functional homologs include SEQ ID NO: 140, 142, 144, 146, and 147. In some cases, a functional homolog of SEQ ID NO: 138 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 138.

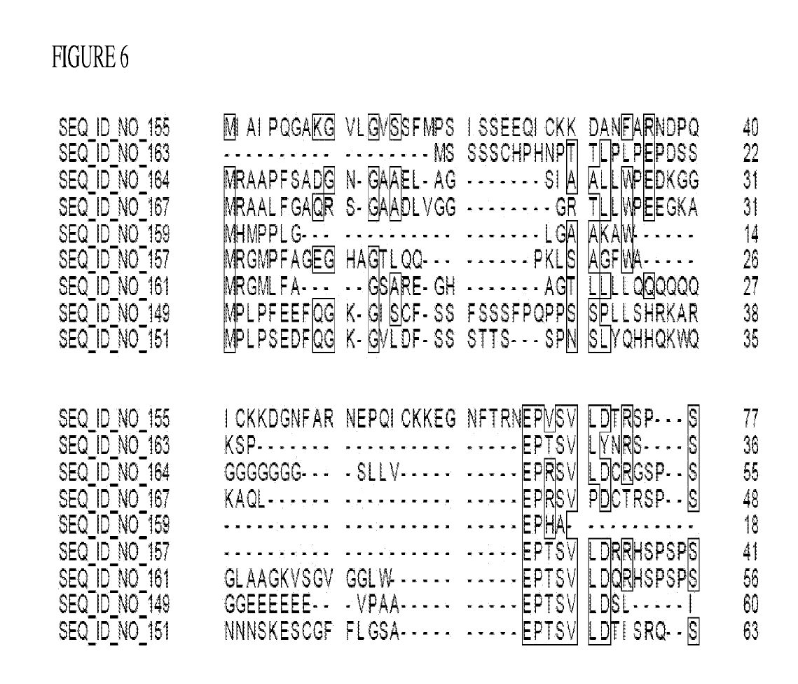

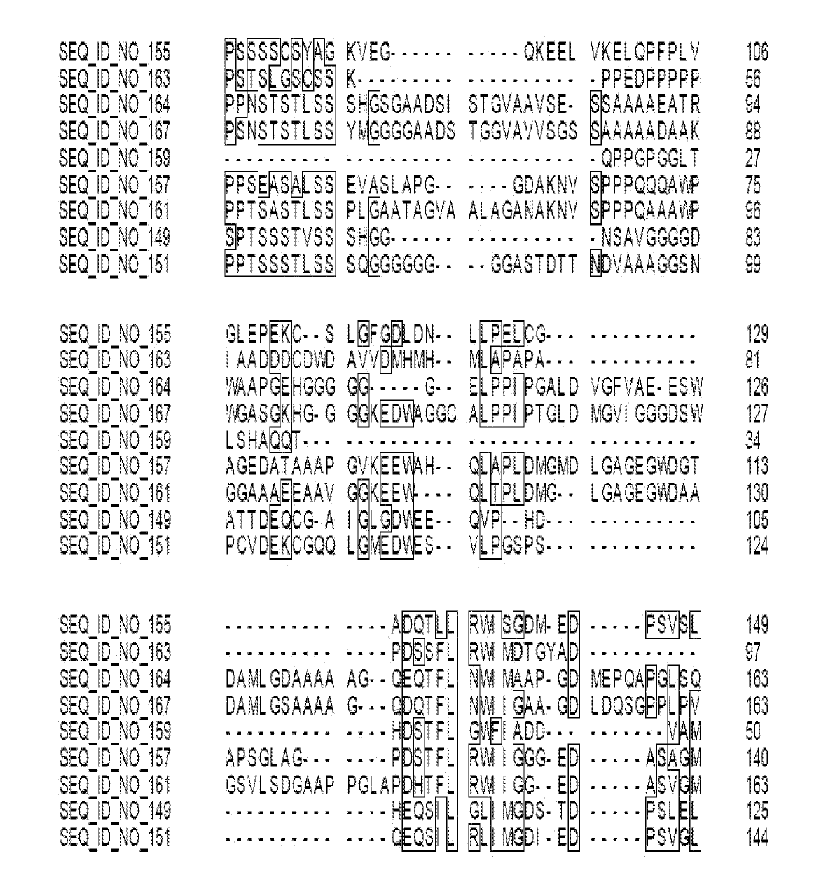

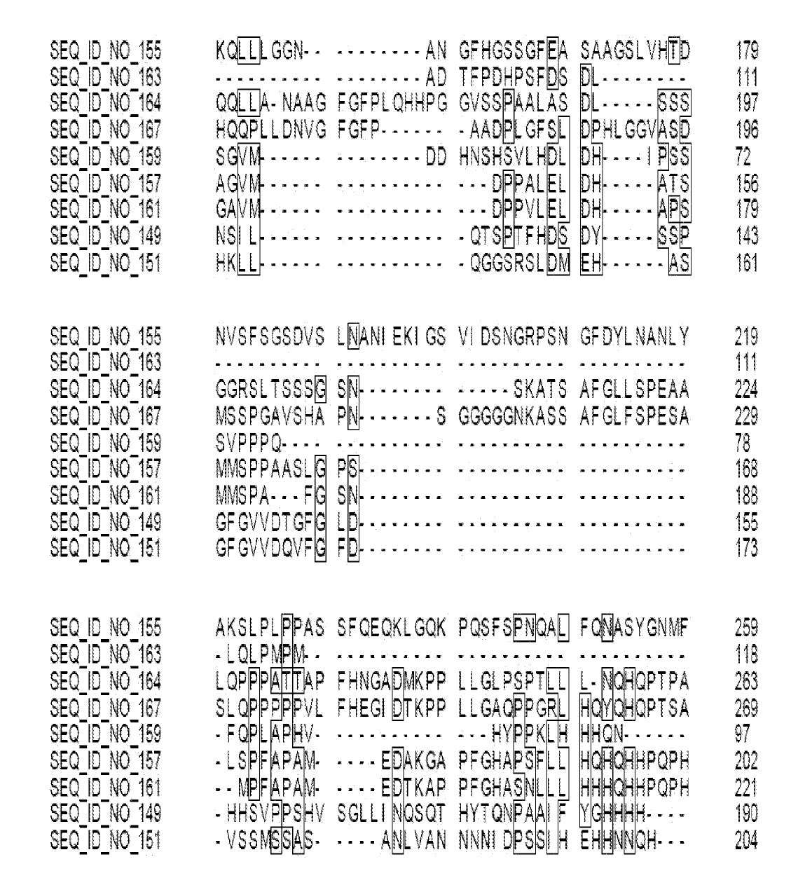

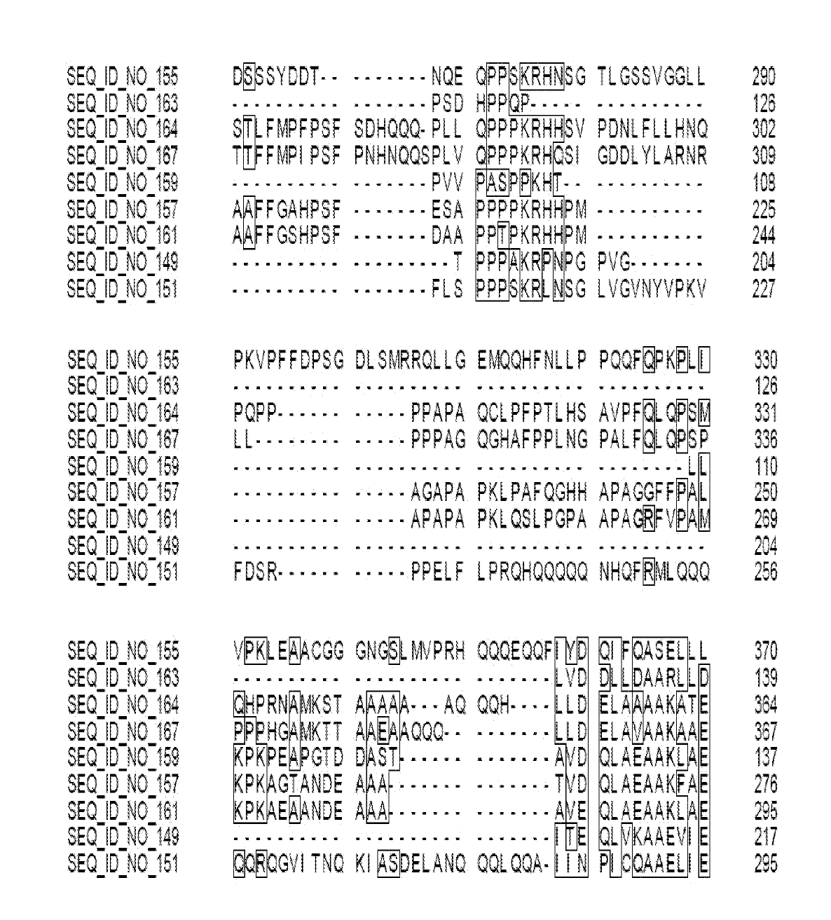

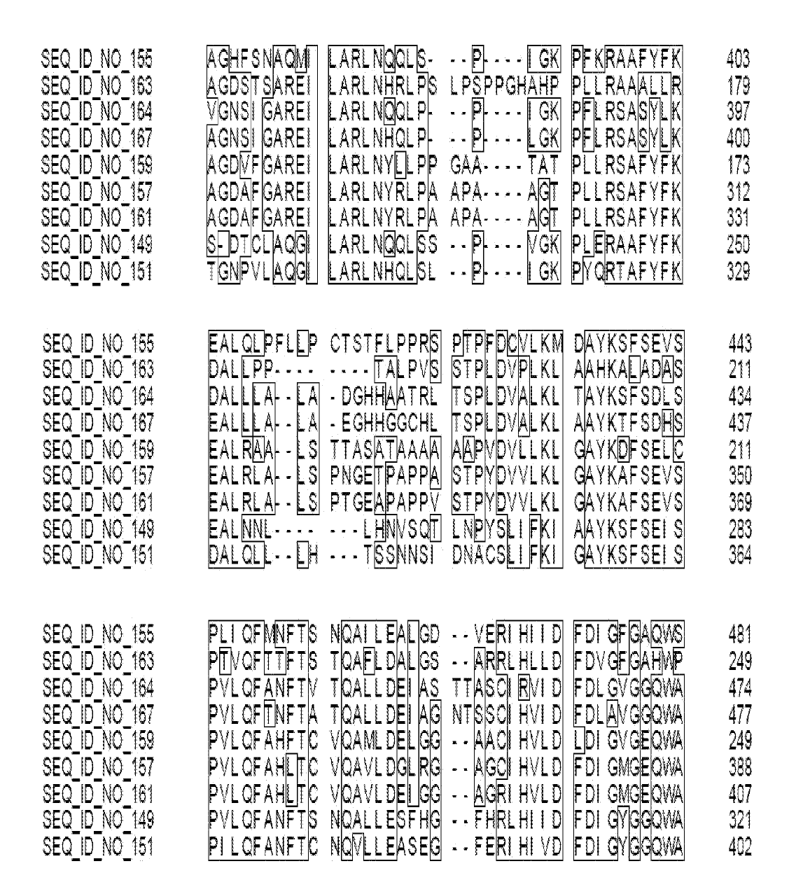

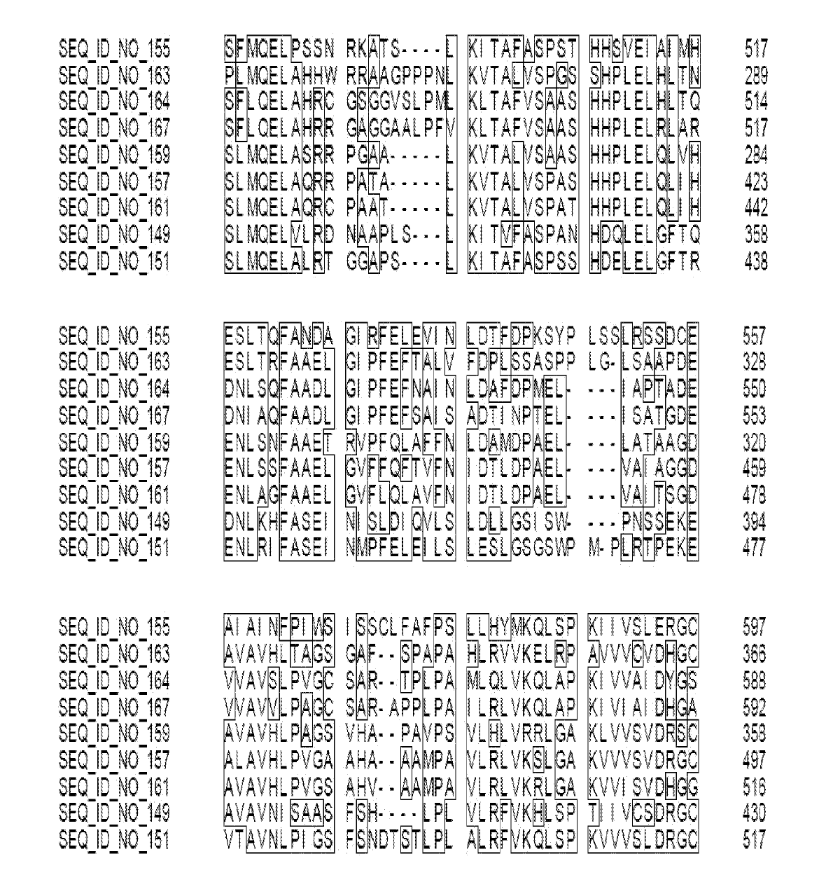

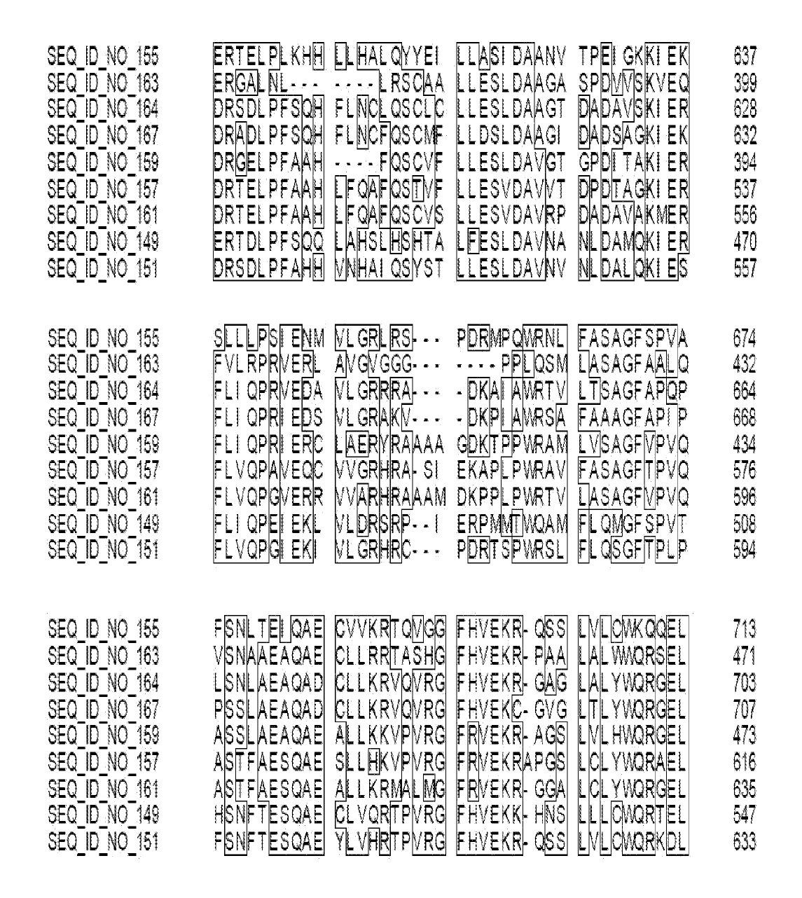

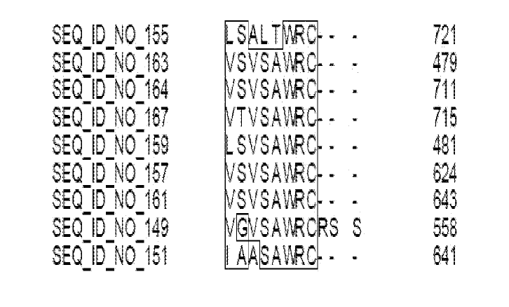

Examples of amino acid sequences of functional homologs of the polypeptide set forth in SEQ ID NO: 149 are provided in FIG. 6 and in the Sequence Listing. Such functional homologs include SEQ ID NO: 151, 155, 157, 159, 161, 163, 164, and 167. In some cases, a functional homolog of SEQ ID NO: 149 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 149.

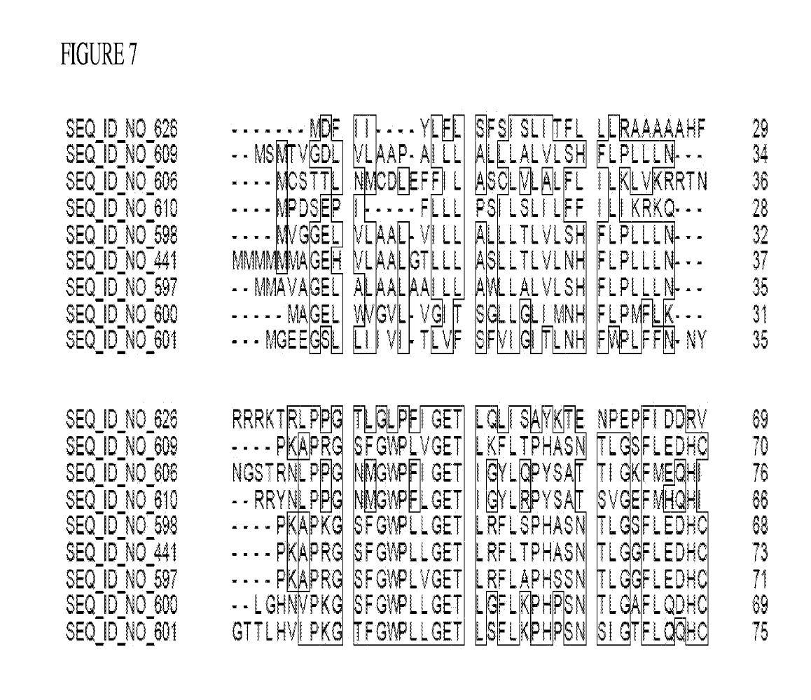

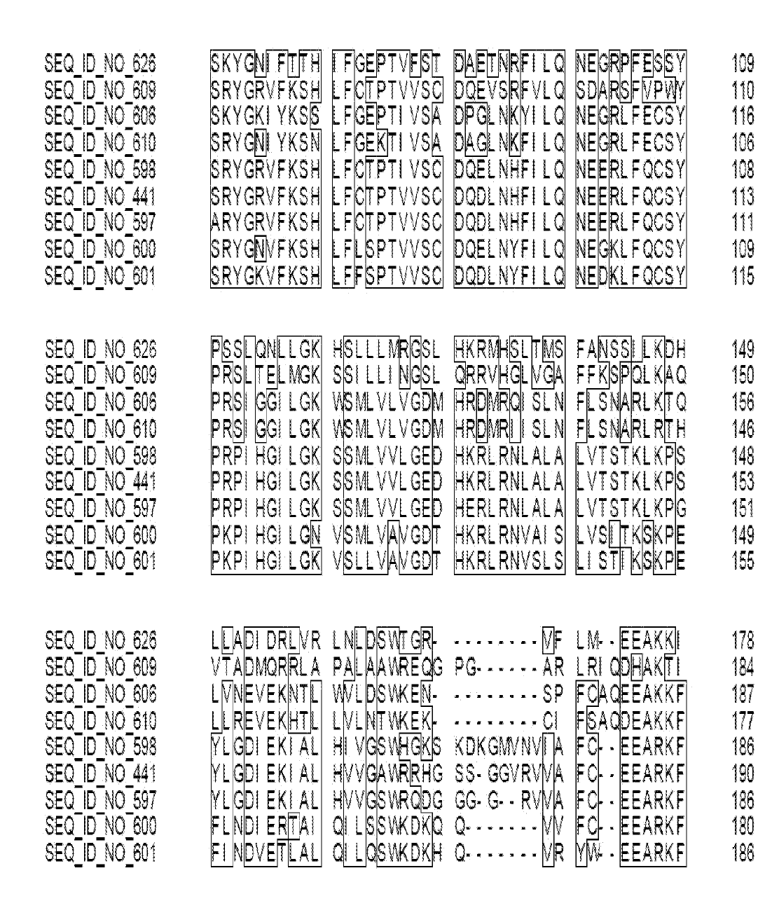

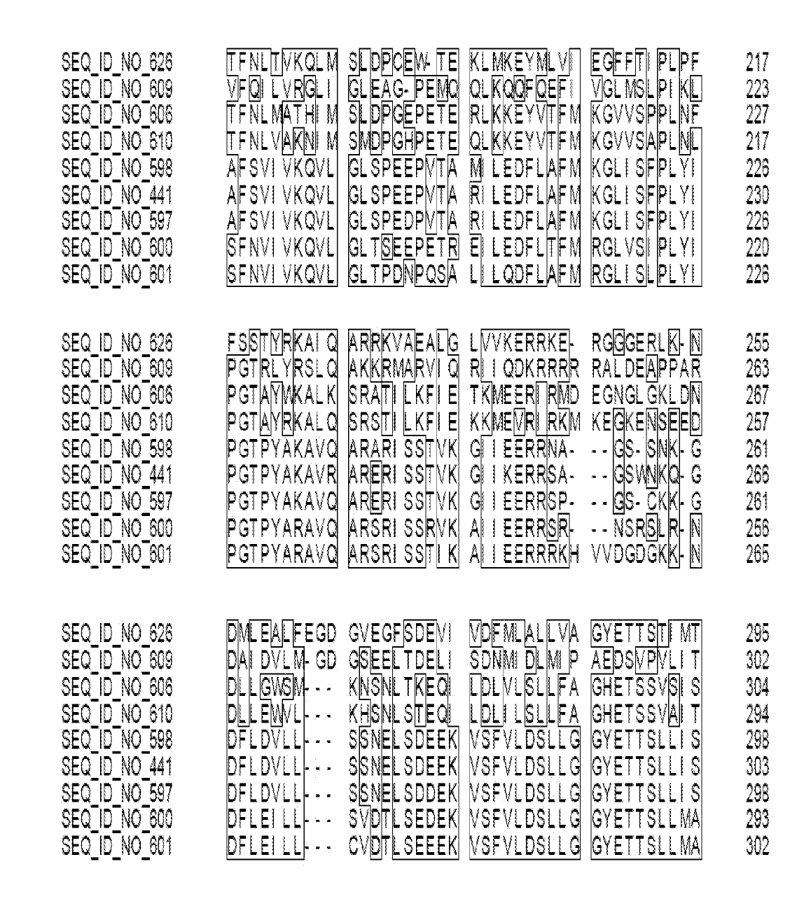

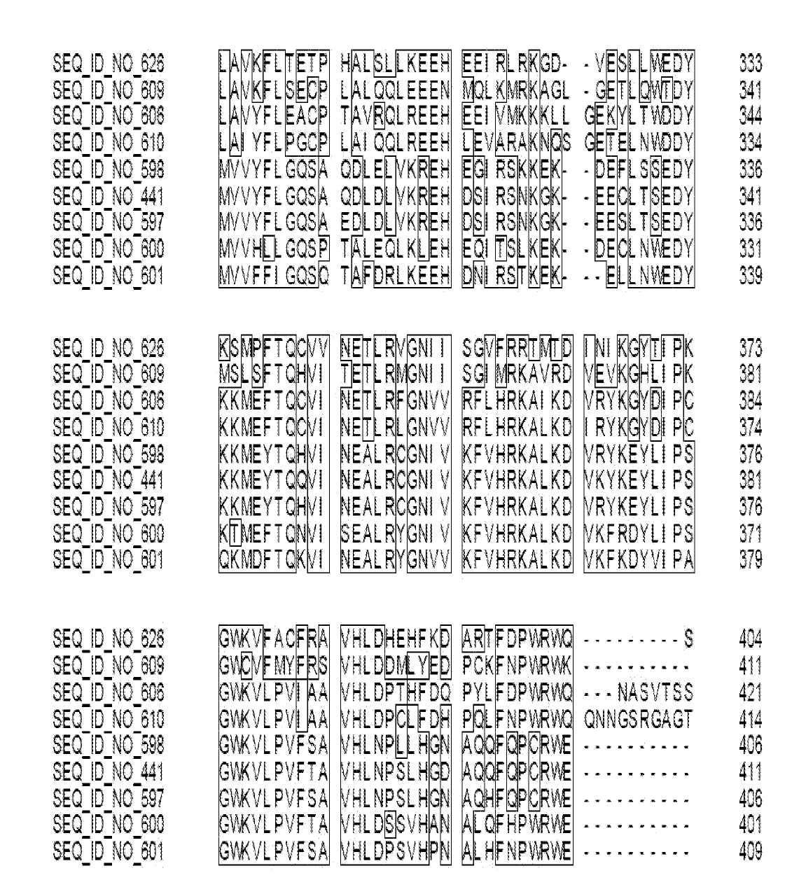

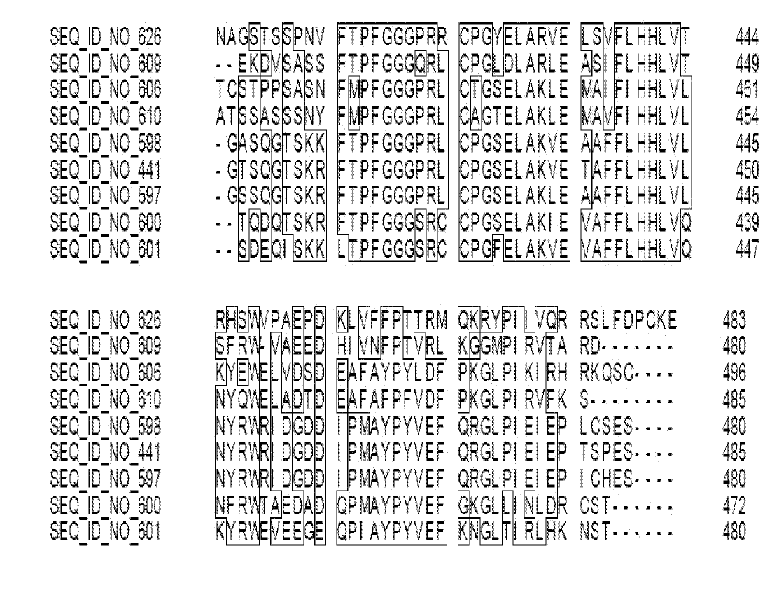

Examples of amino acid sequences of functional homologs of the polypeptide set forth in SEQ ID NO: 441 are provided in FIG. 7 and in the Sequence Listing. Such functional homologs include SEQ ID NO: 442, 444, 445, 446, 447, 449, 450, 451, 452, 453, 454, 456, 457, 458, 459, 460, 461, 462, 463, 464, 465, 467, 468, 469, 470, 471, 472, 473, 474, 475, 477, 479, 480, 481, 482, 484, 486, 487, 489, 490, 491, 492, 493, 494, 496, 498, 500, 502, 503, 504, 505, 506, 507, 508, 509, 511, 512, 513, 514, 516, 517, 518, 519, 520, 522, 523, 524, 525, 527, 529, 531, 532, 533, 534, 536, 537, 538, 539, 540, 541, 543, 544, 546, 547, 549, 550, 551, 552, 553, 555, 556, 557, 559, 560, 562, 563, 564, 565, 566, 567, 569, 571, 572, 574, 576, 577, 578, 580, 582, 583, 585, 586, 588, 590, 591, 592, 593, 594, 595, 597, 598, 600, 601, 603, 605, 606, 608, 609, 610, 611, 613, 614, 615, 616, 617, 618, 620, 622, 624, 625, 626, 627, 629, 631, 633, 635, 637, 639, 640, 641, 642, 643, 644, 646, 648, 650, 651, 653, 654, 655, 657, 659, 660, 661, 662, 663, 664, 665, 666, 668, 669, 671, 673, 675, 677, 678, 680, 682, 684, 686, 688, and 690. In some cases, a functional homolog of SEQ ID NO: 441 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 441.

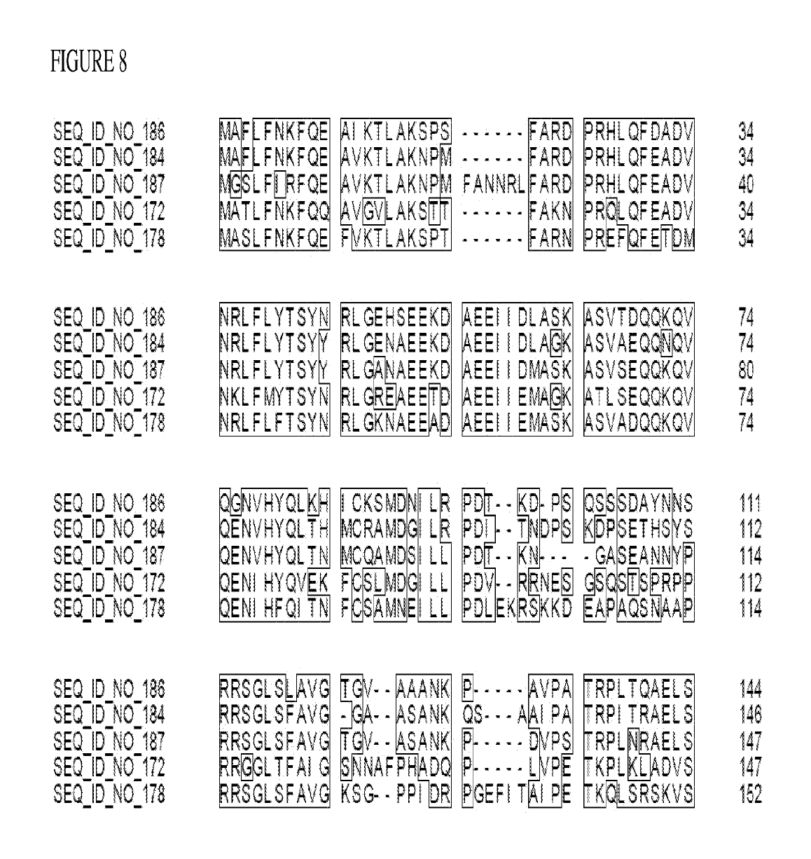

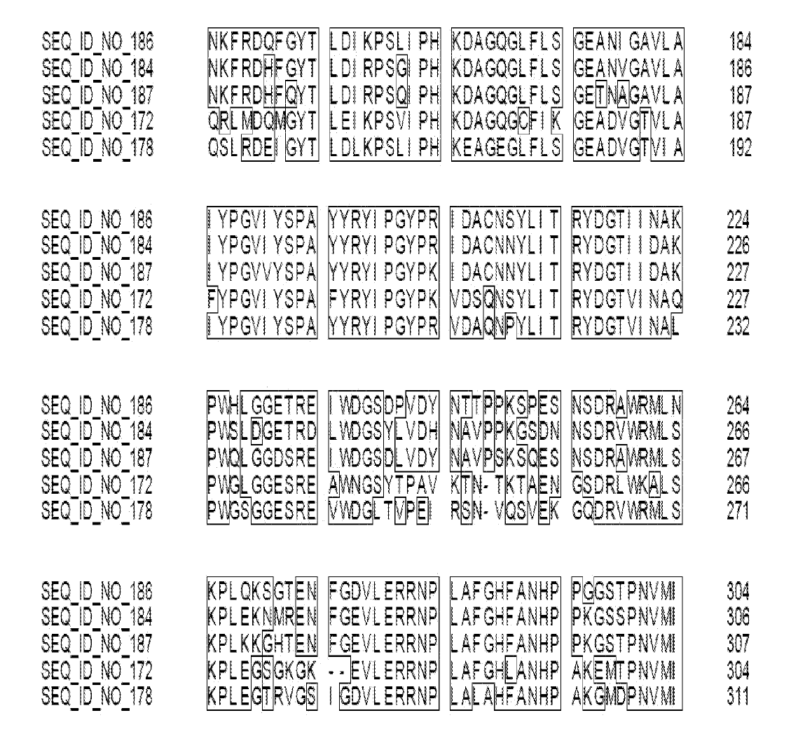

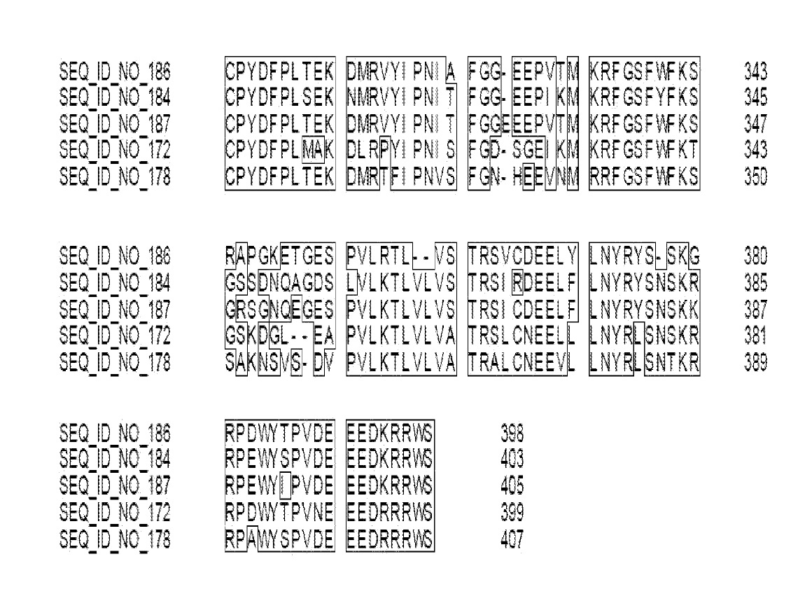

Examples of amino acid sequences of functional homologs of the polypeptide set forth in SEQ ID NO: 172 are provided in FIG. 8 and in the Sequence Listing. Such functional homologs include SEQ ID NO: 174, 175, 178, 179, 180, 182, 184, 186, 187, 188, 189, 191, 192, 194, 196, and 198. In some cases, a functional homolog of SEQ ID NO: 172 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 172.

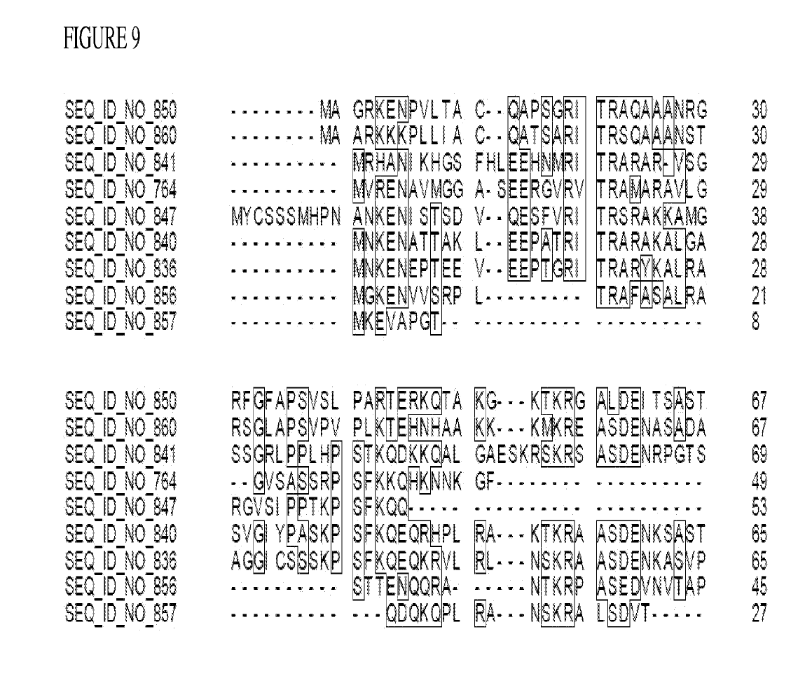

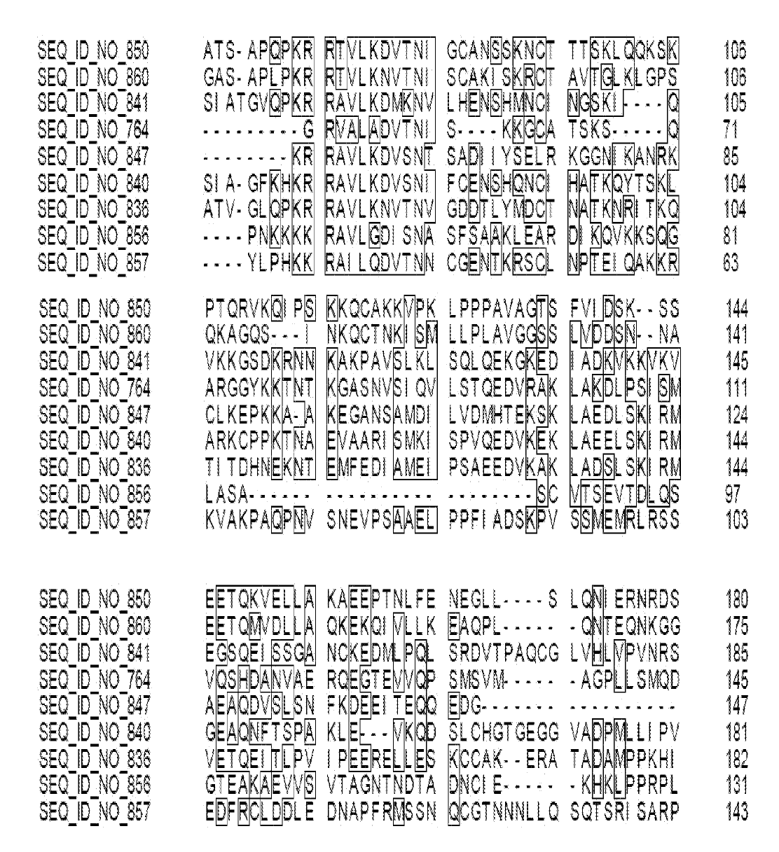

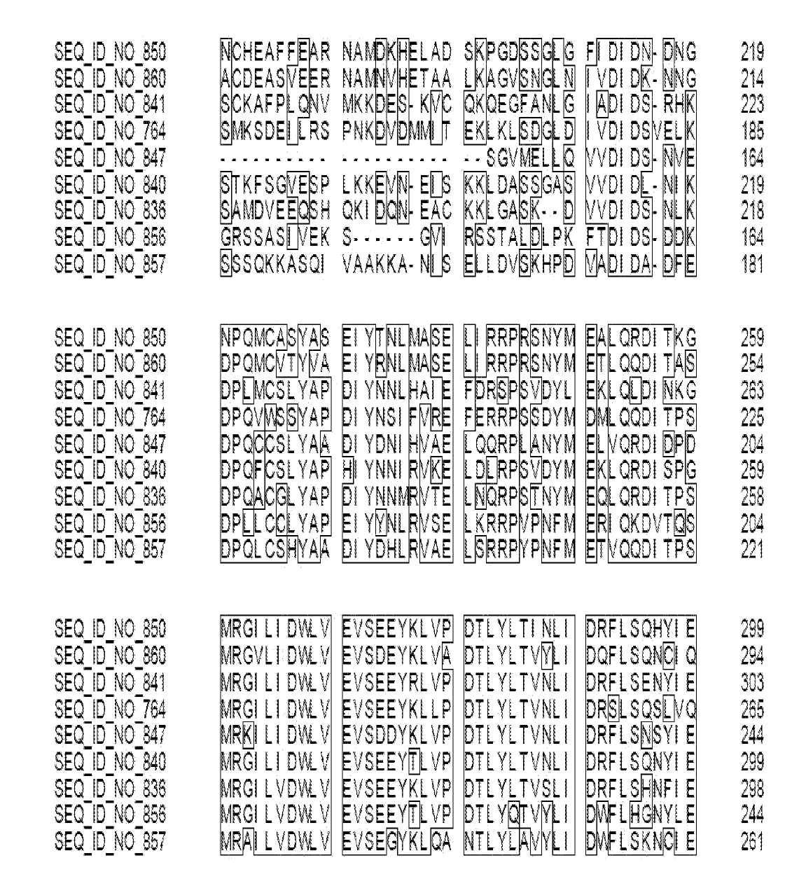

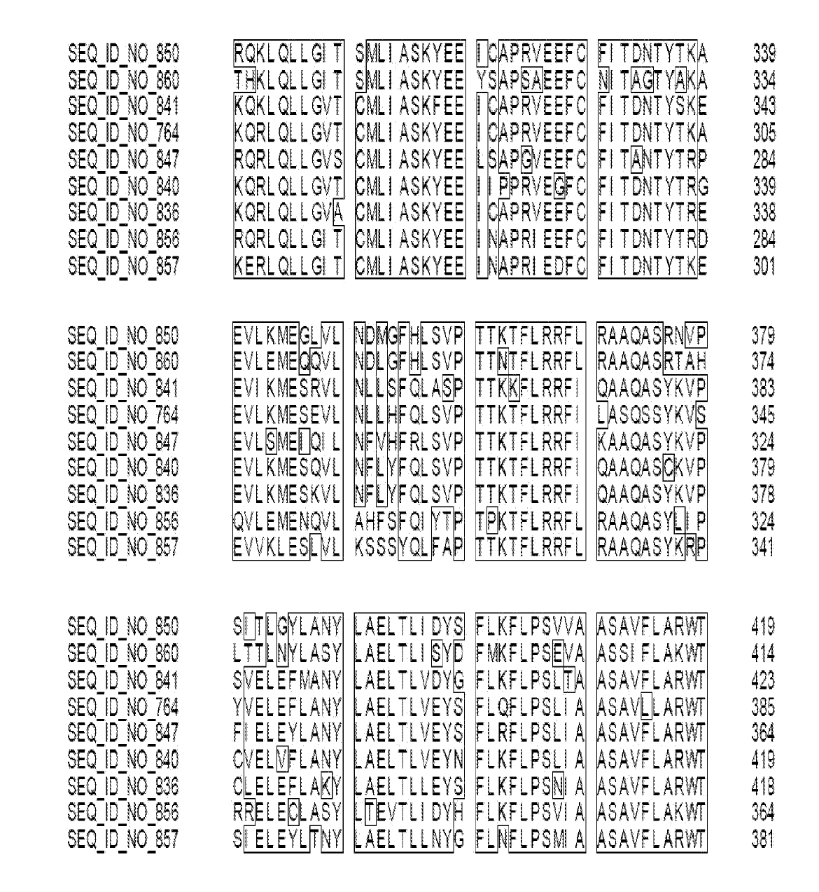

Examples of amino acid sequences of functional homologs of the polypeptide set forth in SEQ ID NO: 764 are provided in FIG. 9 and in the Sequence Listing. Such functional homologs include SEQ ID NO: 765, 766, 767, 769, 770, 771, 773, 774, 775, 776, 777, 779, 780, 781, 782, 784, 785, 786, 788, 790, 792, 793, 795, 796, 797, 798, 801, 802, 804, 805, 808, 809, 810, 812, 813, 814, 815, 816, 817, 818, 819, 820, 822, 824, 825, 826, 827, 829, 832, 834, 836, 838, 840, 841, 843, 845, 846, 847, 848, 849, 850, 851, 852, 854, 855, 856, 857, 858, 860, 861, 862, 863, 864, 867, 870, 871, 872, 874, 876, 878, 880, 882, 884, 886, 888, and 890. In some cases, a functional homolog of SEQ ID NO: 764 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 764.

Examples of amino acid sequences of functional homologs of the polypeptide set forth in SEQ ID NO: 398 are provided in FIG. 10 and in the Sequence Listing. Such functional homologs include SEQ ID NO: 392, 394, 396, 400, 401, 402, 403, 405, 406, 407, 408, 410, 411, 412, 414, 416, 417, 418, 419, 421, 422, 424, 426, 428, 430, 431, 432, 433, 434, 436, 437, and 438. In some cases, a functional homolog of SEQ ID NO: 398 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 398.

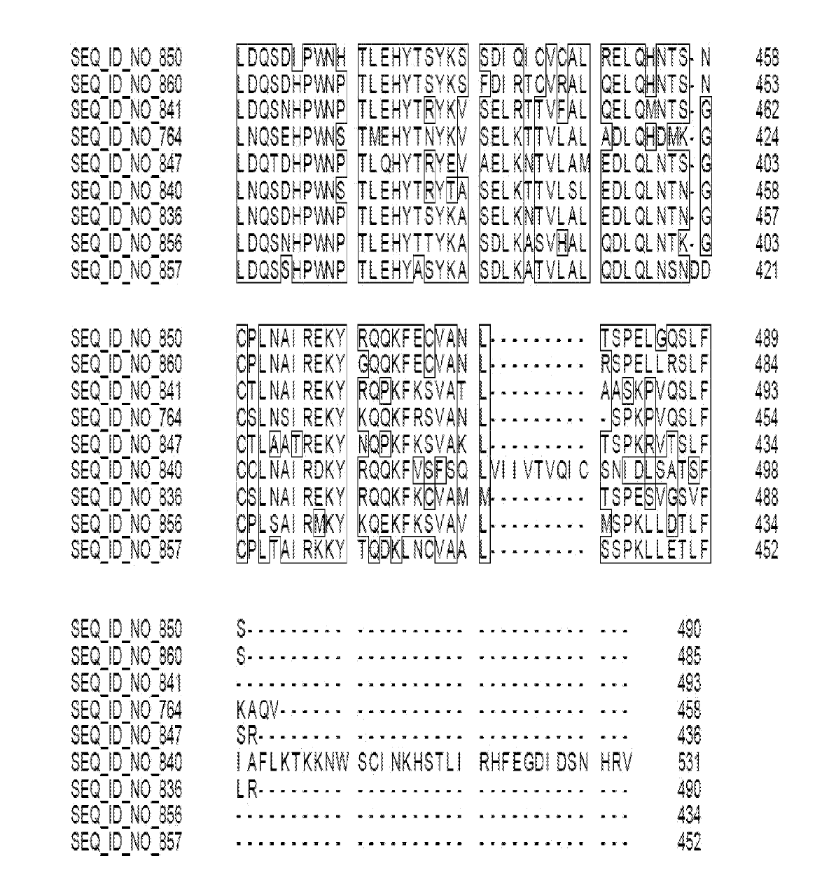

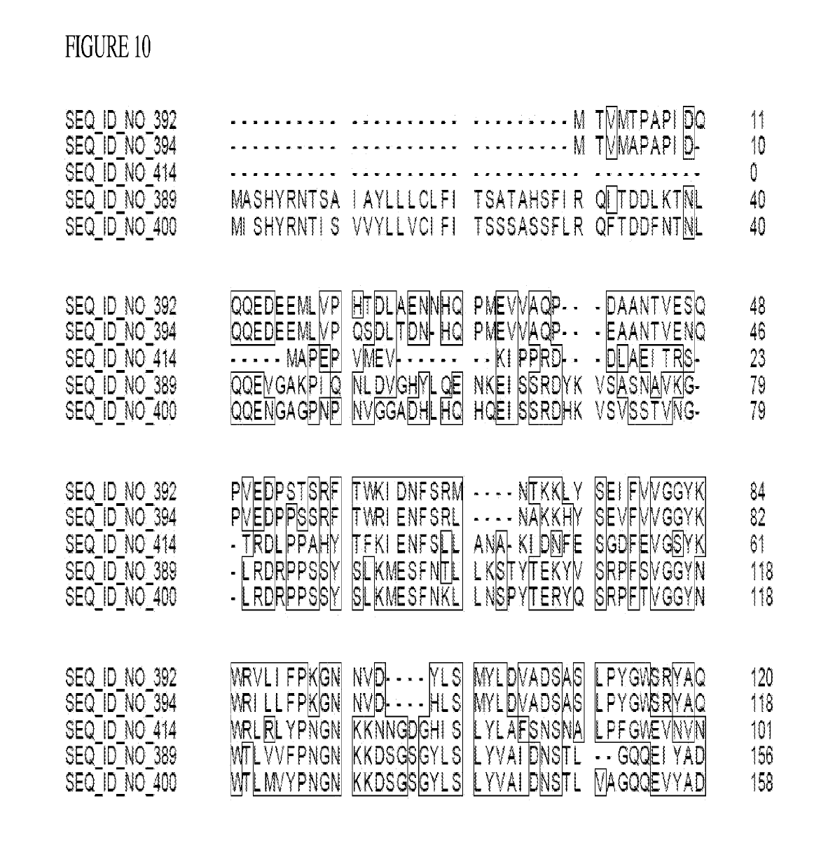

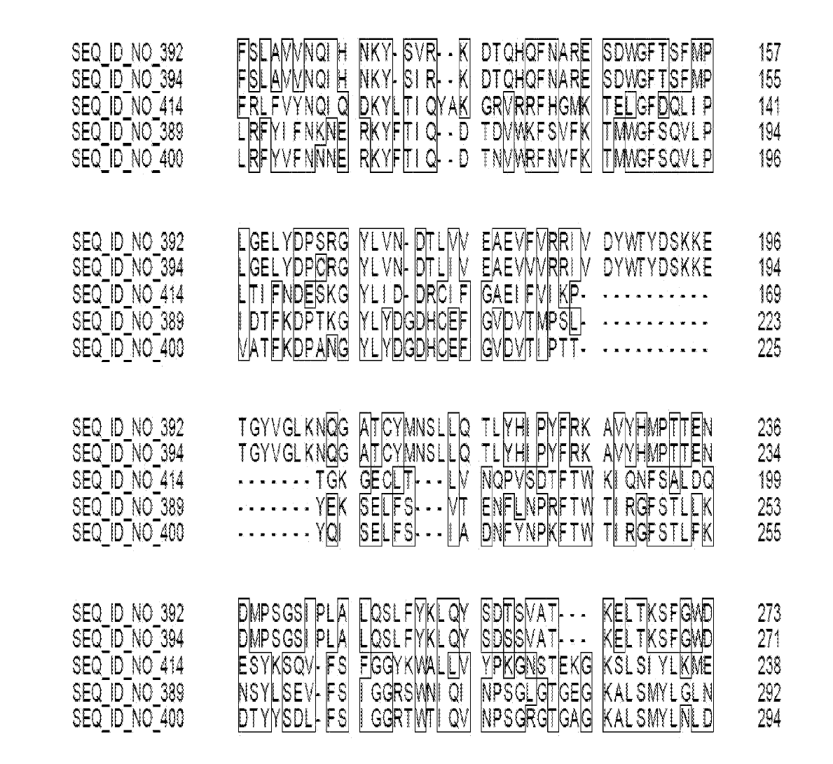

Examples of amino acid sequences of functional homologs of the polypeptide set forth in SEQ ID NO: 316 are provided in FIG. 11 and in the Sequence Listing. Such functional homologs include SEQ ID NO: 318, 319, 320, 321, 323, 324, 331, 333, 335, 337, 339, 340, 344, 346, 348, 350, 351, 353, 355, 356, 357, 359, 361, 363, 365, 367, 369, 374, 375, 377, 378, 380, 382, 384, and 386. In some cases, a functional homolog of SEQ ID NO: 316 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 316.

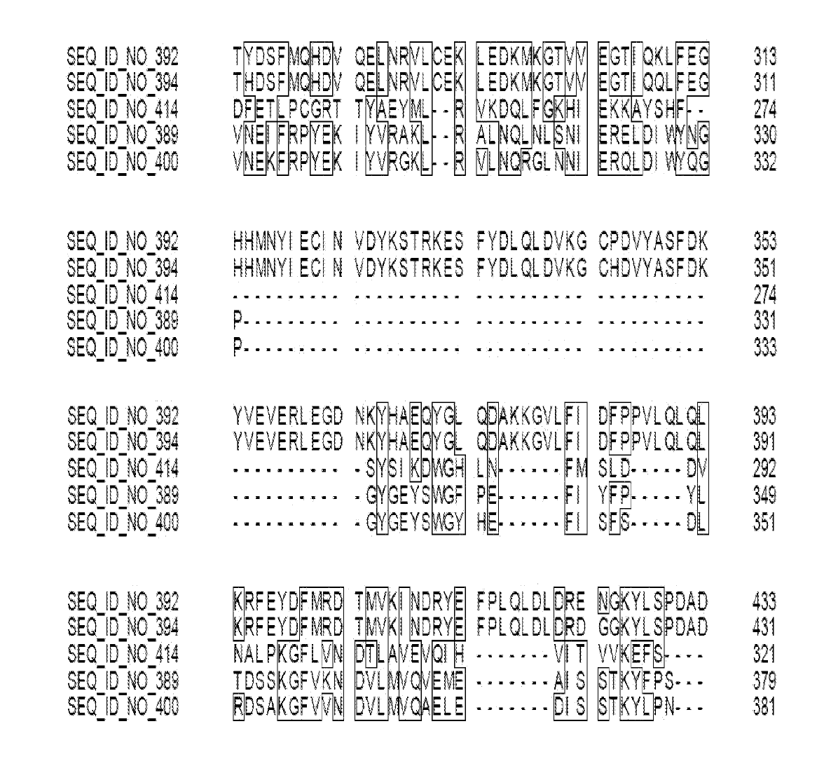

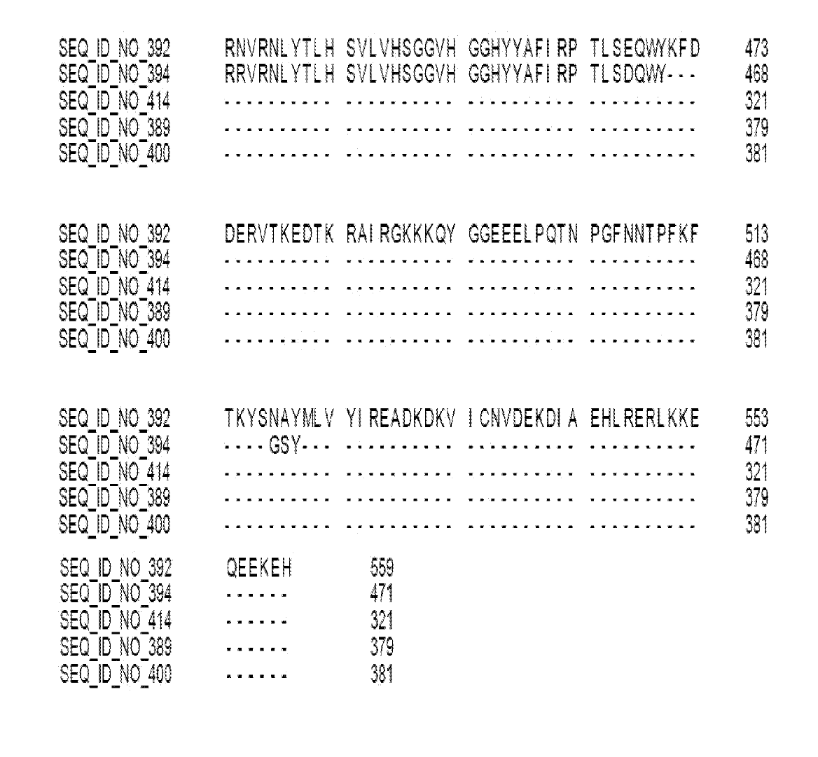

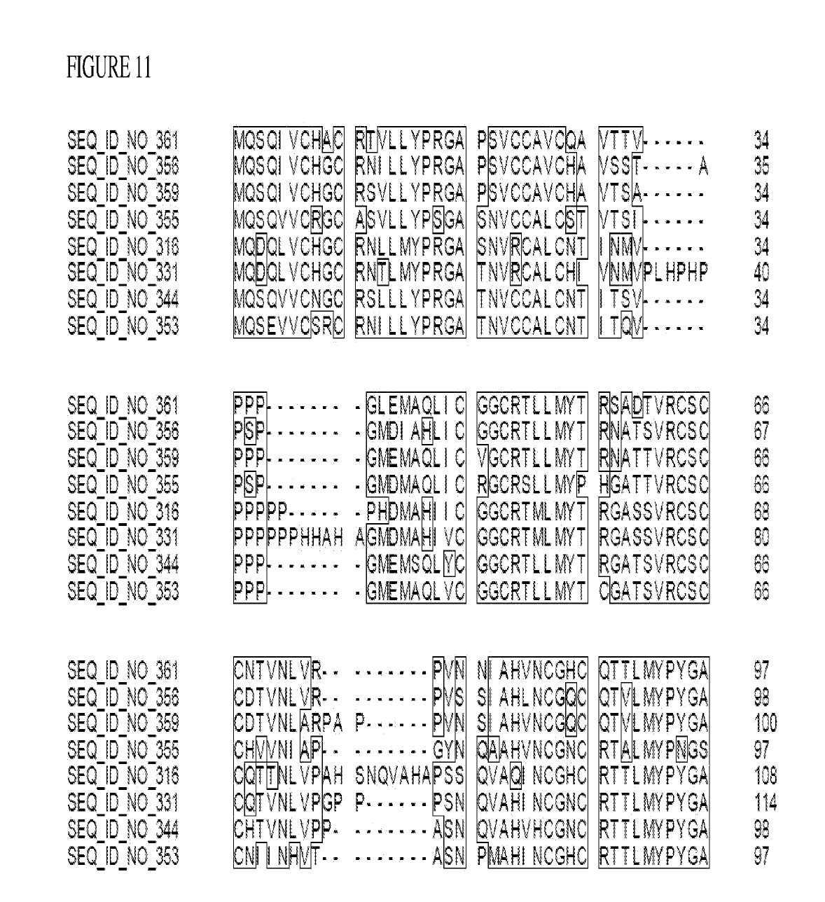

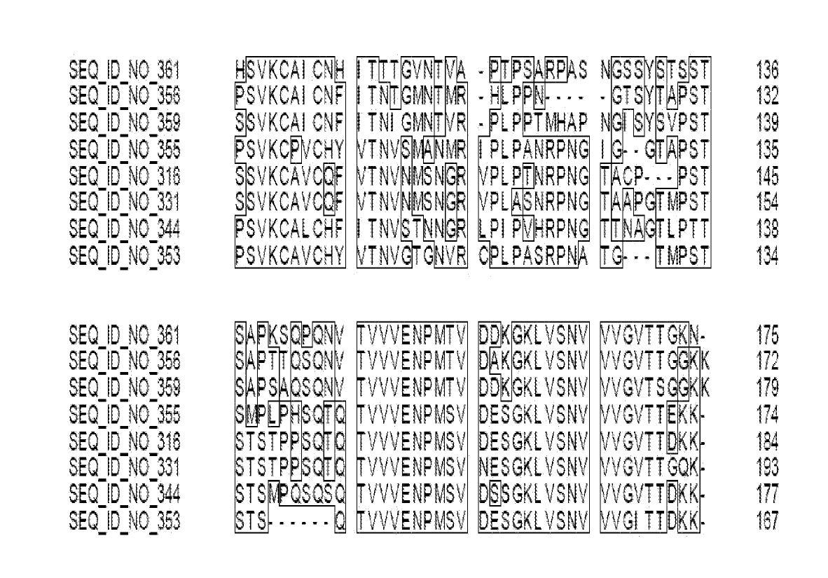

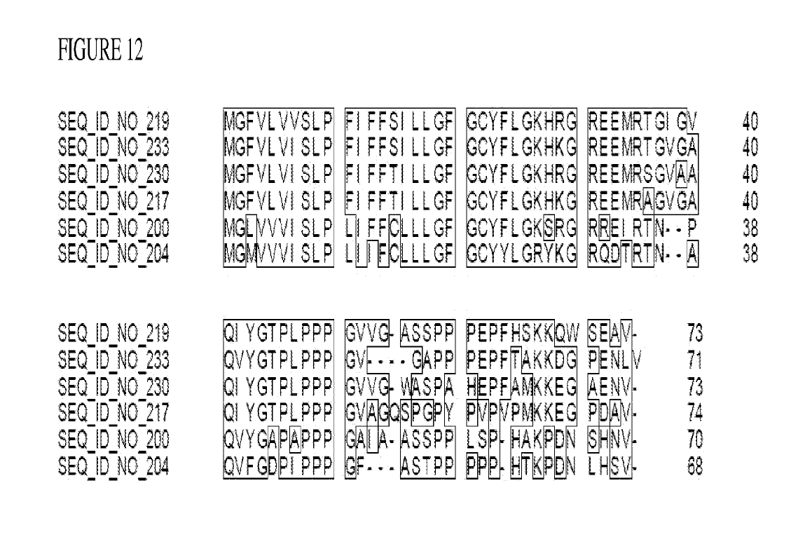

Examples of amino acid sequences of functional homologs of the polypeptide set forth in SEQ ID NO: 200 are provided in FIG. 12 and in the Sequence Listing. Such functional homologs include SEQ ID NO: 202, 204, 206, 208, 211, 217, 219, 223, 225, 226, 228, 230, 232, 233, 235, and 237. In some cases, a functional homolog of SEQ ID NO: 200 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 200.

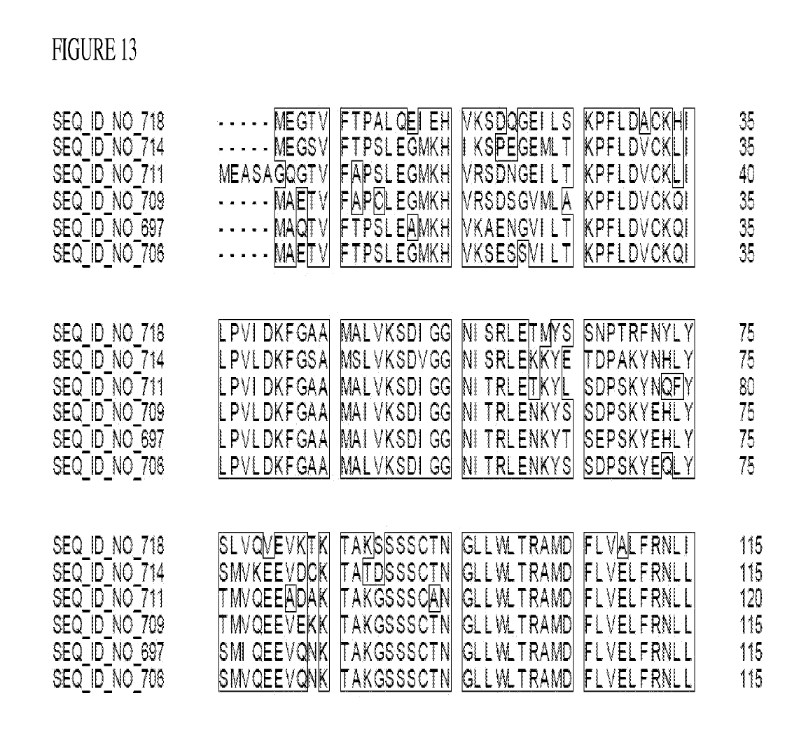

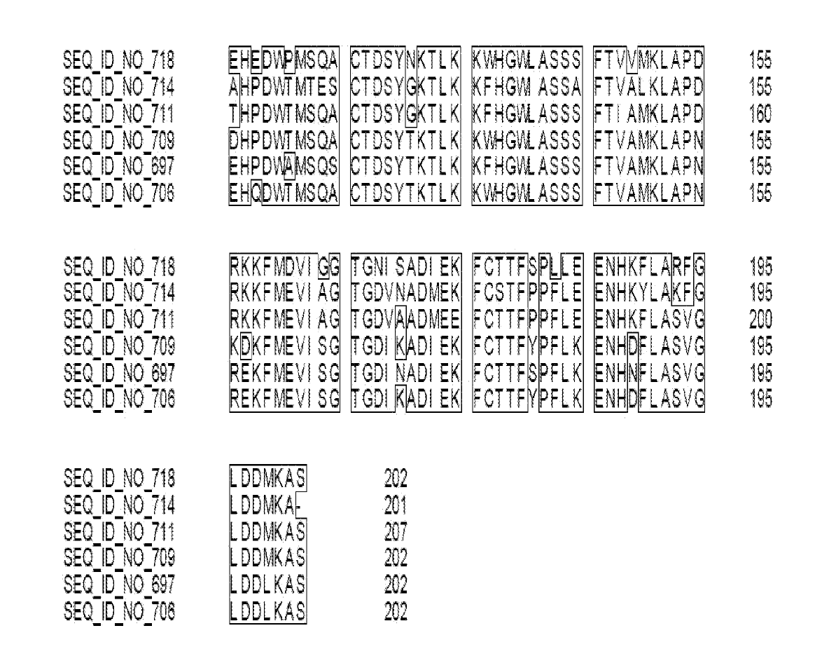

Examples of amino acid sequences of functional homologs of the polypeptide set forth in SEQ ID NO: 697 are provided in FIG. 13 and in the Sequence Listing. Such functional homologs include SEQ ID NO: 699, 703, 706, 707, 709, 711, 712, 714, 718, 720, 722, 723, 726, 728, 730, 732, 733, 735, 737, 739, 740, 741, 742, 743, 745, 746, 747, 749, 751, 752, 753, 755, 757, 759, and 761. In some cases, a functional homolog of SEQ ID NO: 697 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 697.

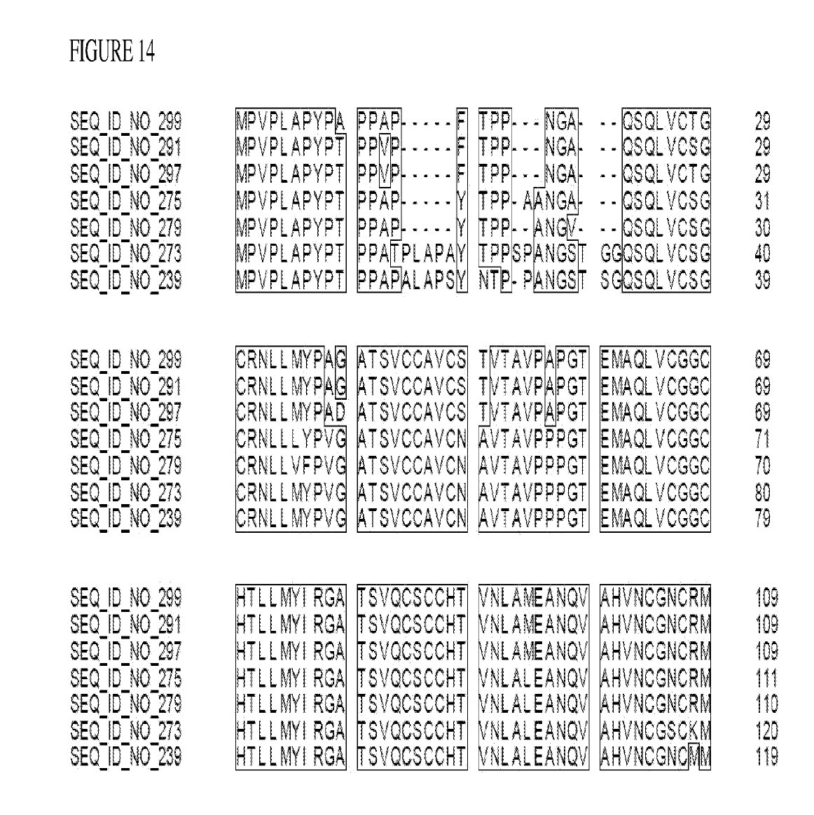

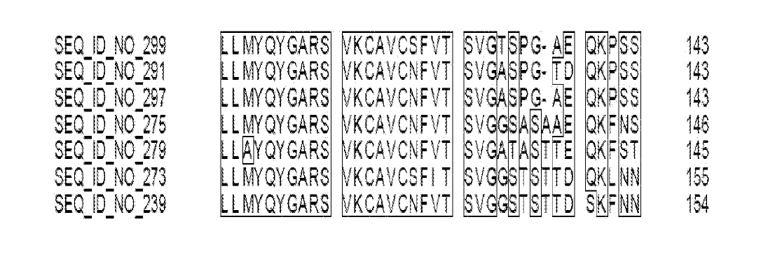

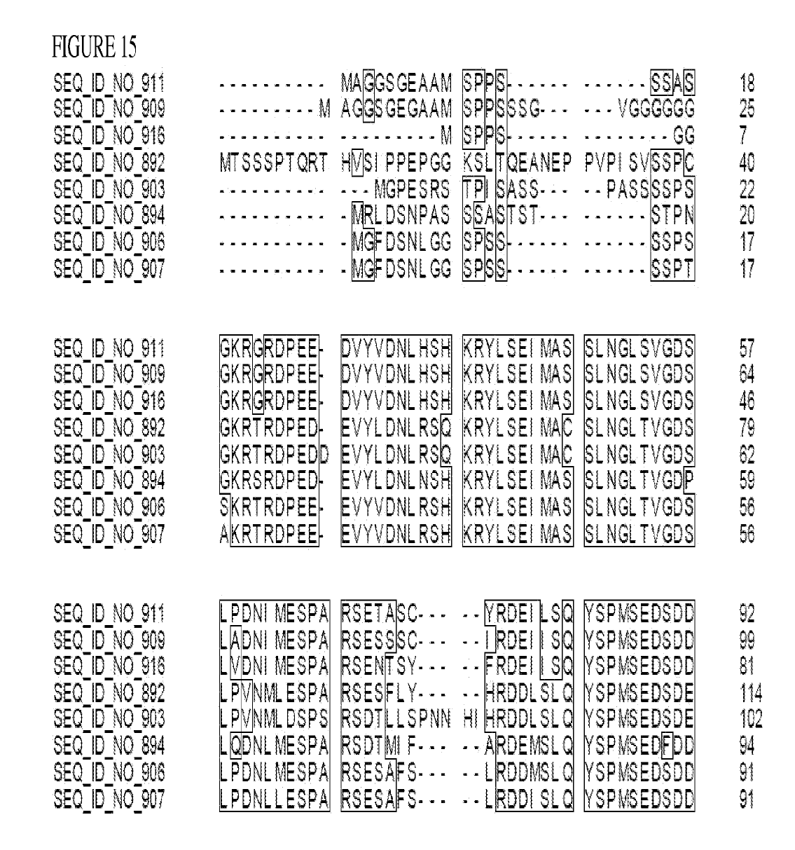

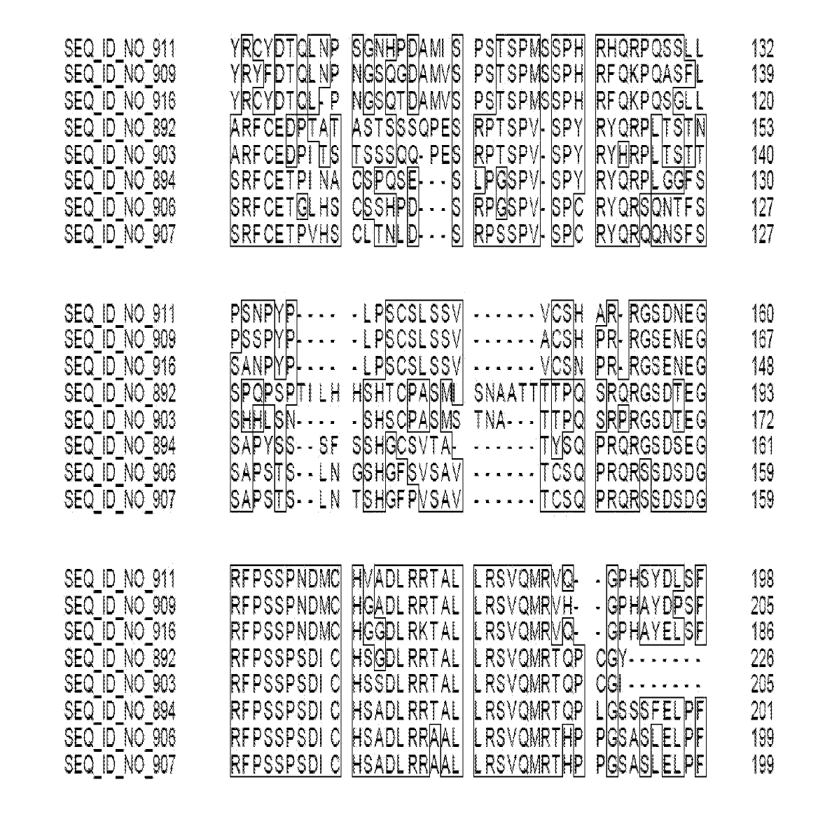

Examples of amino acid sequences of functional homologs of the polypeptide set forth in SEQ ID NO: 239 are provided in FIG. 14 and in the Sequence Listing. Such functional homologs include SEQ ID NO: 241, 243, 245, 247, 249, 250, 251, 253, 255, 257, 259, 261, 262, 264, 266, 270, 271, 272, 273, 275, 279, 281, 282, 284, 286, 288, 290, 291, 292, 293, 294, 295, 297, 299, 301, 302, 304, 306, and 308. In some cases, a functional homolog of SEQ ID NO: 239 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 239.

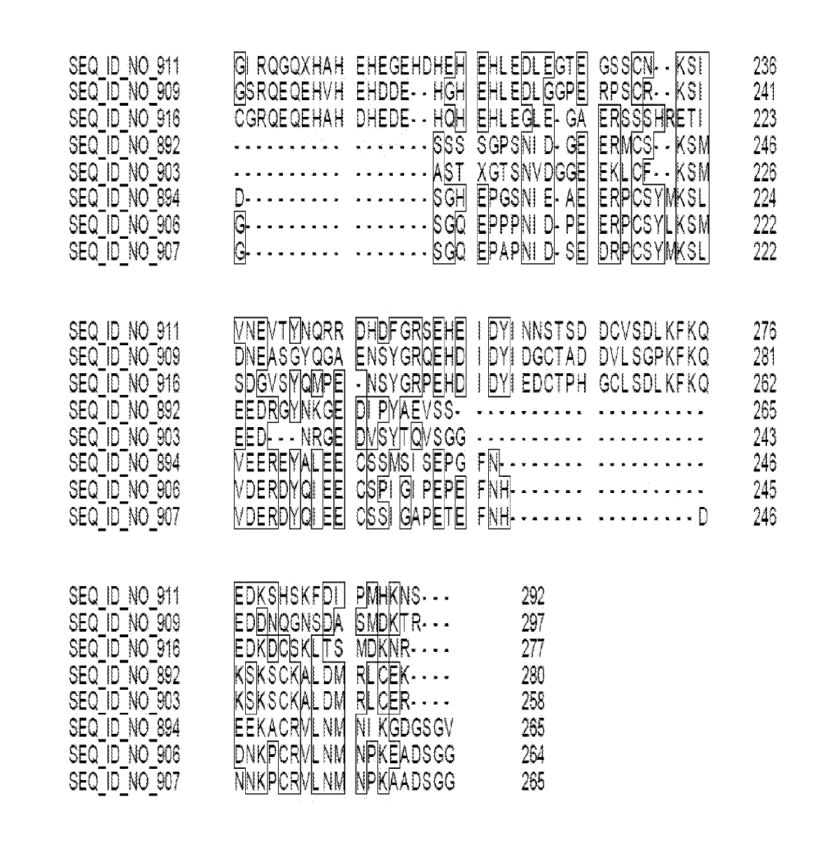

Examples of amino acid sequences of functional homologs of the polypeptide set forth in SEQ ID NO: 892 are provided in FIG. 15 and in the Sequence Listing. Such functional homologs include SEQ ID NO: 894, 896, 898, 901, 903, 905, 906, 907, 909, 911, 913, 914, 915, 916, 918, and 920. In some cases, a functional homolog of SEQ ID NO: 892 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 892.

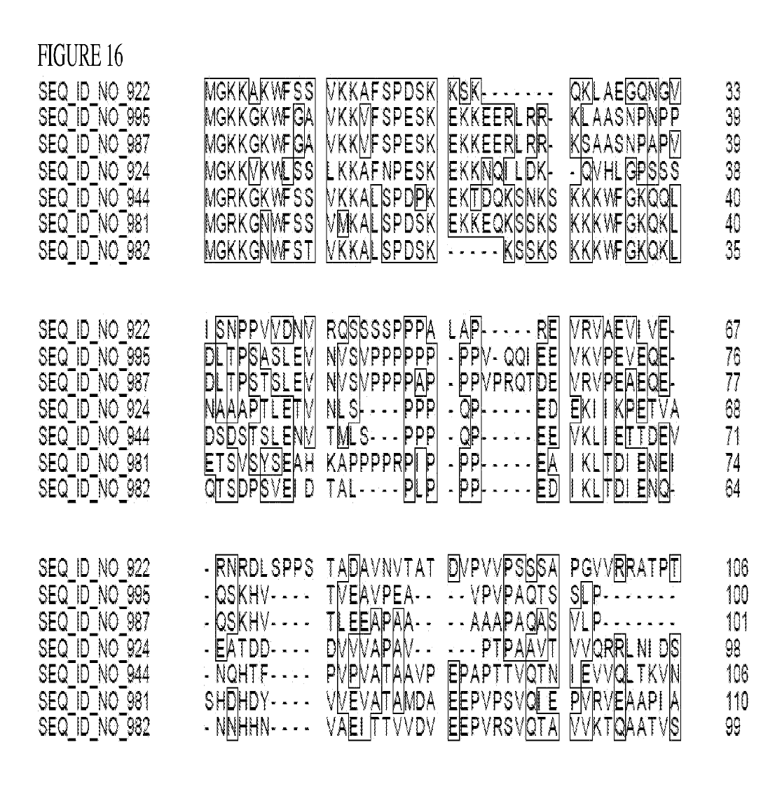

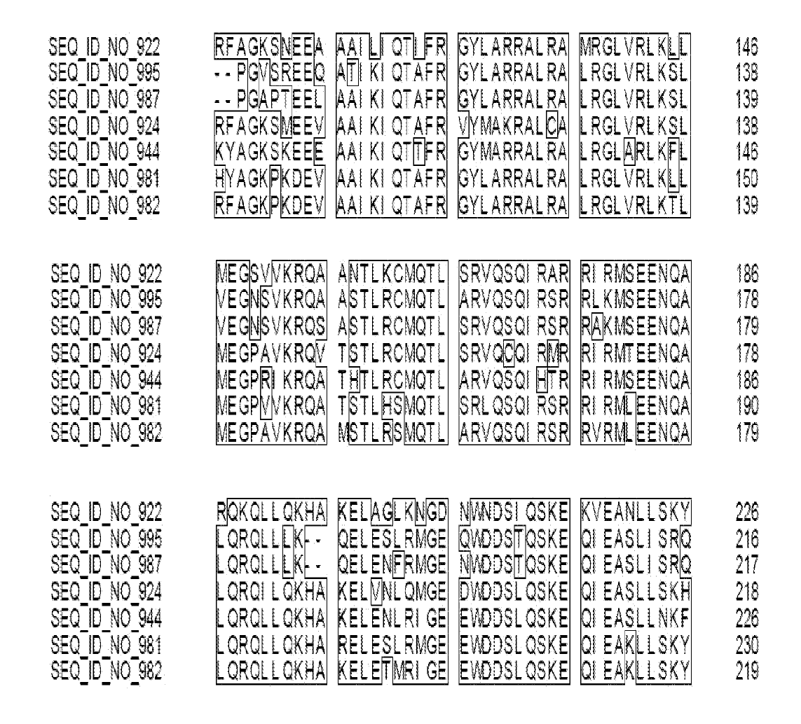

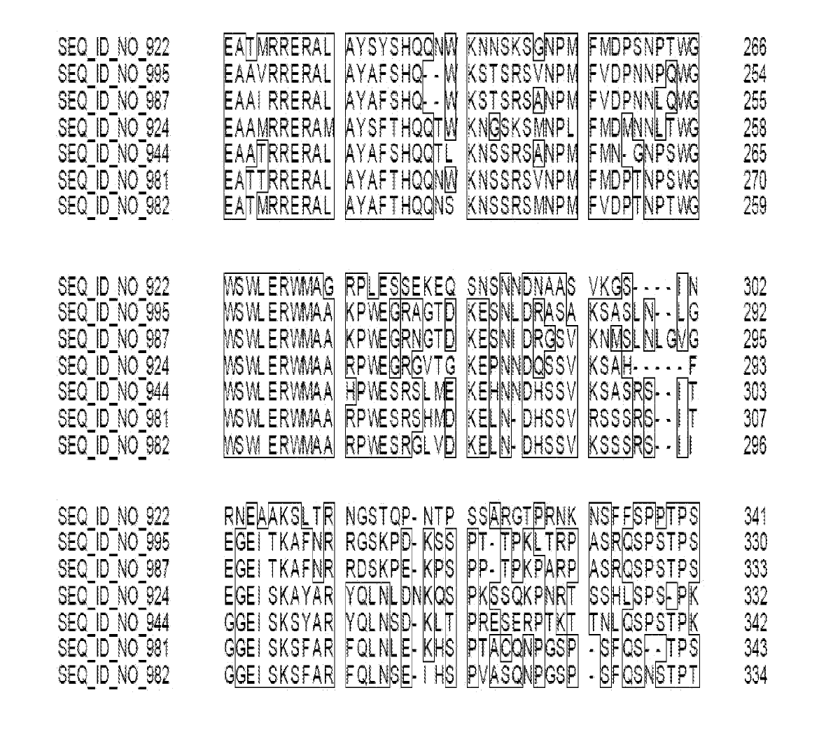

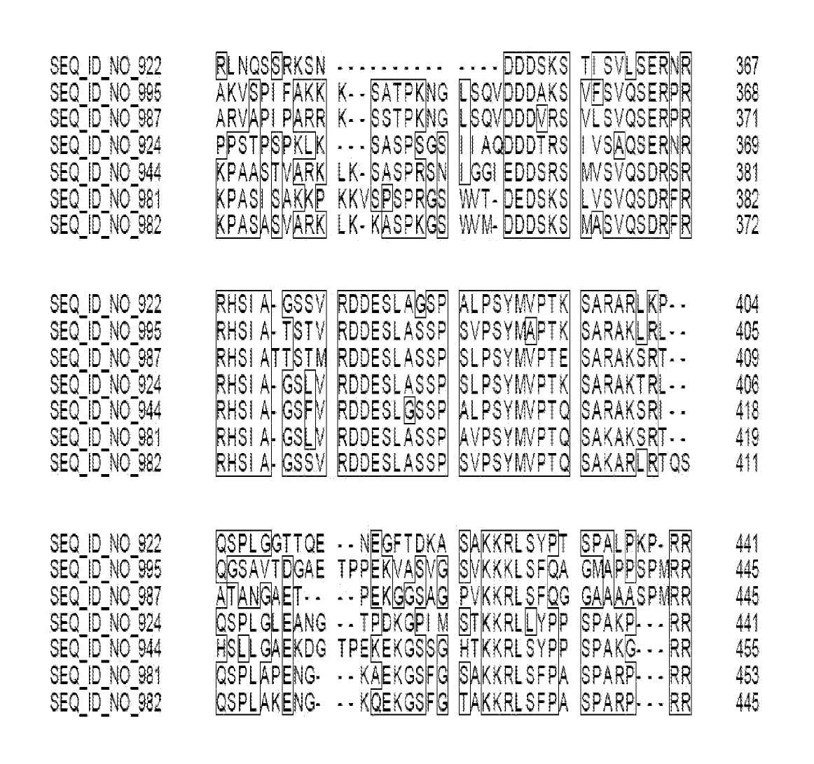

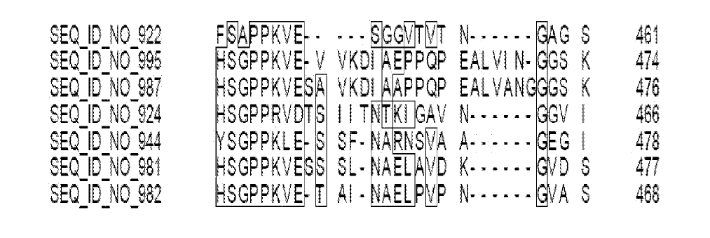

Examples of amino acid sequences of functional homologs of the polypeptide set forth in SEQ ID NO: 922 are provided in FIG. 16 and in the Sequence Listing. Such functional homologs include SEQ ID NO: 724, 806, 831, 866, 868, 869, 899, 924, 926, 928, 930, 932, 934, 936, 938, 940, 942, 944, 946, 948, 950, 952, 954, 956, 958, 960, 962, 964, 966, 968, 970, 972, 974, 975, 976, 977, 978, 979, 981, 982, 983, 985, 987, 989, 991, 993, 994, 995, and 996. In some cases, a functional homolog of SEQ ID NO: 922 has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO: 922.

The identification of conserved regions in a salinity and/or oxidative stress tolerance-modulating polypeptide facilitates production of variants of salinity and/or oxidative stress tolerance-modulating polypeptides. Variants of salinity and/or oxidative stress tolerance-modulating polypeptides typically have 10 or fewer conservative amino acid substitutions within the primary amino acid sequence, e.g., 7 or fewer conservative amino acid substitutions, 5 or fewer conservative amino acid substitutions, or between 1 and 5 conservative substitutions. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys, His and Arg; exchange of the hydrophobic residues Leu, Ile, Val; interchange of the small residues Ala, Ser, Thr, Met and Gly; and replacements among the aromatic residues Phe, Trp, Tyr. Guidance concerning which amino acid changes are likely to be phenotypically silent under particular circumstances is found in Bowie et al., Science 247:1306-1310 (1990).

A useful variant polypeptide can be constructed based on one of the alignments set forth in FIGS. 1 thru 16. Such a polypeptide includes the conserved regions, arranged in the order depicted in the Figure from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

C. Functional Homologues Identified by HMM

In some embodiments, useful salinity and/or oxidative stress tolerance-modulating polypeptides include those that fit a Hidden Markov Model based on the polypeptides set forth in any one of FIGS. 1-16. A Hidden Markov Model (HMM) is a statistical model of a consensus sequence for a group of functional homologs. See, Durbin et al., Biological Sequence Analysis: Probabilistic Models of Proteins and Nucleic Acids, Cambridge University Press, Cambridge, UK (1998). An HMM is generated by the program HMMER 2.3.2 with default program parameters, using the sequences of the group of functional homologs as input. The multiple sequence alignment is generated by ProbCons (Do et al., Genome Res., 15(2):330-40 (2005)) version 1.11 using a set of default parameters: -c, --consistency REPS of 2; -ir, --iterative-refinement REPS of 100; -pre, --pre-training REPS of 0. ProbCons is a public domain software program provided by Stanford University.

The default parameters for building an HMM (hmmbuild) are as follows: the default “architecture prior” (archpri) used by MAP architecture construction is 0.85, and the default cutoff threshold (idlevel) used to determine the effective sequence number is 0.62. HMMER 2.3.2 was released Oct. 3, 2003 under a GNU general public license, and is available from various sources on the World Wide Web Hmmbuild outputs the model as a text file.

The HMM for a group of functional homologs can be used to determine the likelihood that a candidate salinity and/or oxidative stress tolerance-modulating polypeptide sequence is a better fit to that particular HMM than to a null HMM generated using a group of sequences that are not structurally or functionally related. The likelihood that a subject polypeptide sequence is a better fit to an HMM than to a null HMM is indicated by the HMM bit score, a number generated when the candidate sequence is fitted to the HMM profile using the HMMER hmmsearch program. The following default parameters are used when running hmmsearch: the default E-value cutoff (E) is 10.0, the default bit score cutoff (T) is negative infinity, the default number of sequences in a database (Z) is the real number of sequences in the database, the default E-value cutoff for the per-domain ranked hit list (domE) is infinity, and the default bit score cutoff for the per-domain ranked hit list (domT) is negative infinity. A high HMM bit score indicates a greater likelihood that the subject sequence carries out one or more of the biochemical or physiological function(s) of the polypeptides used to generate the HMM. A high HMM bit score is at least 15, and often is higher.

As those of skill in the art would appreciate, the HMM scores provided in the sequence listing are merely exemplary. Since multiple sequence alignment algorithms, such as ProbCons, can only generate near-optimal results, slight variations of the model can arise due to factors such as the order in which sequences are processed for alignment. Nevertheless, HMM score variability is minor, and so the HMM scores in the sequence listing are representative of models made with the respective sequences.

The salinity and/or oxidative stress-modulating polypeptides discussed below fit the indicated HMM with an HMM bit score greater than 15 (e.g., greater than 20, 30, 40, 50, 60, 70, 80, 90, 100, 200, 300, 400, or 500). In some embodiments, the HMM bit score of a salinity and/or oxidative stress-modulating polypeptide discussed below is about 50%, 60%, 70%, 80%, 90%, or 95% of the HMM bit score of a functional homolog provided in the Sequence Listing. In some embodiments, a salinity and/or oxidative stress-modulating polypeptide discussed below fits the indicated HMM with an HMM bit score greater than 15, and has a domain indicative of a salinity and/or oxidative stress-modulating polypeptide. In some embodiments, a salinity and/or oxidative stress-modulating polypeptide discussed below fits the indicated HMM with an HMM bit score greater than 15, and has 85% or greater sequence identity (e.g., 75%, 80%, 85%, 90%, 95%, or 100% sequence identity) to an amino acid sequence shown in any one of FIGS. 1 thru 16 or to an amino acid sequence correlated in the Sequence Listing to a any one of FIGS. 1 thru 16.

In the Sequence Listing polypeptides are provided that have HMM bit scores greater than 170 when fitted to an HMM generated from the amino acid sequences set forth in FIG. 1. Such polypeptides include SEQ ID NO: 2, 4, 6, 7, and 9.

In the Sequence Listing polypeptides are provided that have HMM bit scores greater than 255 when fitted to an HMM generated from the amino acid sequences set forth in FIG. 2. Such polypeptides include SEQ ID NO: 11, 13, 15, 17, 19, 21, 23, 25, 27, 29, and 30.

In the Sequence Listing polypeptides are provided that have HMM bit scores greater than 65 or 100 when fitted to an HMM generated from the amino acid sequences set forth in FIG. 3. Such polypeptides include SEQ ID NO: 102, 103, 104, 106, 108, 110, 112, 114, 115, 116, 117, 118, 119, 120, 122, 124, 125, 127, 128, 129, 130, 132, 134, and 136.

In the Sequence Listing polypeptides are provided that have HMM bit scores greater than 175 when fitted to an HMM generated from the amino acid sequences set forth in FIG. 4. Such polypeptides include SEQ ID NO: 32, 33, 35, 37, 38, 40, 42, 44, 46, 48, 49, 51, 53, 54, 56, 58, 60, 62, 63, 64, 65, 66, 67, 68, 69, 71, 73, 75, 77, 79, 81, 83, 85, 87, 89, 91, 93, 95, 97, 98, 99, and 100.

In the Sequence Listing polypeptides are provided that have HMM bit scores greater than 1095 when fitted to an HMM generated from the amino acid sequences set forth in FIG. 5. Such polypeptides include SEQ ID NO: 138, 140, 142, 144, 146, and 147.

In the Sequence Listing polypeptides are provided that have HMM bit scores greater than 535 when fitted to an HMM generated from the amino acid sequences set forth in FIG. 6. Such polypeptides include SEQ ID NO: 149, 151, 155, 157, 159, 161, 163, 164, and 167.

In the Sequence Listing polypeptides are provided that have HMM bit scores greater than 120 when fitted to an HMM generated from the amino acid sequences set forth in FIG. 7. Such polypeptides include SEQ ID NO: 441, 442, 444, 445, 446, 447, 449, 450, 451, 452, 453, 454, 456, 457, 458, 459, 460, 461, 462, 463, 464, 465, 467, 468, 469, 470, 471, 472, 473, 474, 475, 477, 479, 480, 481, 482, 484, 486, 487, 489, 490, 491, 492, 493, 494, 496, 498, 500, 502, 503, 504, 505, 506, 507, 508, 509, 511, 512, 513, 514, 516, 517, 518, 519, 520, 522, 523, 524, 525, 527, 529, 531, 532, 533, 534, 536, 537, 538, 539, 540, 541, 543, 544, 546, 547, 549, 550, 551, 552, 553, 555, 556, 557, 559, 560, 562, 563, 564, 565, 566, 567, 569, 571, 572, 574, 576, 577, 578, 580, 582, 583, 585, 586, 588, 590, 591, 592, 593, 594, 595, 597, 598, 600, 601, 603, 605, 606, 608, 609, 610, 611, 613, 614, 615, 616, 617, 618, 620, 622, 624, 625, 626, 627, 629, 631, 633, 635, 637, 639, 640, 641, 642, 643, 644, 646, 648, 650, 651, 653, 654, 655, 657, 659, 660, 661, 662, 663, 664, 665, 666, 668, 669, 671, 673, 675, 677, 678, 680, 682, 684, 686, 688, and 690.

In the Sequence Listing polypeptides are provided that have HMM bit scores greater than 455 when fitted to an HMM generated from the amino acid sequences set forth in FIG. 8. Such polypeptides include SEQ ID NO: 172, 174, 175, 178, 179, 180, 182, 184, 186, 187, 188, 189, 191, 192, 194, 196, and 198.

In the Sequence Listing polypeptides are provided that have HMM bit scores greater than 180 when fitted to an HMM generated from the amino acid sequences set forth in FIG. 9. Such polypeptides include SEQ ID NO: 764, 765, 766, 767, 769, 770, 771, 773, 774, 775, 776, 777, 779, 780, 781, 782, 784, 785, 786, 788, 790, 792, 793, 795, 796, 797, 798, 801, 802, 804, 805, 808, 809, 810, 812, 813, 814, 815, 816, 817, 818, 819, 820, 822, 824, 825, 826, 827, 829, 832, 834, 836, 838, 840, 841, 843, 845, 846, 847, 848, 849, 850, 851, 852, 854, 855, 856, 857, 858, 860, 861, 862, 863, 864, 867, 870, 871, 872, 874, 876, 878, 880, 882, 884, 886, 888, and 890.

In the Sequence Listing polypeptides are provided that have HMM bit scores greater than 110 when fitted to an HMM generated from the amino acid sequences set forth in FIG. 10. Such polypeptides SEQ ID NO: 389, 392, 394, 396, 398, 400, 401, 402, 403, 405, 406, 407, 408, 410, 411, 412, 414, 416, 417, 418, 419, 421, 422, 424, 426, 428, 430, 431, 432, 433, 434, 436, 437, and 438.

In the Sequence Listing polypeptides are provided that have HMM bit scores greater than 75 or 280 when fitted to an HMM generated from the amino acid sequences set forth in FIG. 11. Such polypeptides include SEQ ID NO: 316, 318, 319, 320, 321, 323, 324, 331, 333, 335, 337, 339, 340, 344, 346, 348, 350, 351, 353, 355, 356, 357, 359, 361, 363, 365, 367, 369, 374, 375, 377, 378, 380, 382, 384, and 386.

In the Sequence Listing polypeptides are provided that have HMM bit scores greater than 15 or 130 when fitted to an HMM generated from the amino acid sequences set forth in FIG. 12. Such polypeptides include SEQ ID NO: 200, 202, 204, 206, 208, 211, 217, 219, 223, 225, 226, 228, 230, 232, 233, 235, and 237.

In the Sequence Listing polypeptides are provided that have HMM bit scores greater than 70 or 390 when fitted to an HMM generated from the amino acid sequences set forth in FIG. 13. Such polypeptides include SEQ ID NO: 697, 699, 703, 706, 707, 709, 711, 712, 714, 718, 720, 722, 723, 726, 728, 730, 732, 733, 735, 737, 739, 740, 741, 742, 743, 745, 746, 747, 749, 751, 752, 753, 755, 757, 759, and 761.

In the Sequence Listing polypeptides are provided that have HMM bit scores greater than 50 or 340 when fitted to an HMM generated from the amino acid sequences set forth in FIG. 14. Such polypeptides include SEQ ID NO: 239, 241, 243, 245, 247, 249, 250, 251, 253, 255, 257, 259, 261, 262, 264, 266, 270, 271, 272, 273, 275, 279, 281, 282, 284, 286, 288, 290, 291, 292, 293, 294, 295, 297, 299, 301, 302, 304, 306, and 308.

In the Sequence Listing polypeptides are provided that have HMM bit scores greater than 300 when fitted to an HMM generated from the amino acid sequences set forth in FIG. 15. Such polypeptides include SEQ ID NO: 892, 894, 896, 898, 901, 903, 905, 906, 907, 909, 911, 913, 914, 915, 916, 918, and 920.

In the Sequence Listing polypeptides are provided that have HMM bit scores greater than 25 or 550 when fitted to an HMM generated from the amino acid sequences set forth in FIG. 16. Such polypeptides include SEQ ID NO: 724, 806, 831, 866, 868, 869, 899, 922, 924, 926, 928, 930, 932, 934, 936, 938, 940, 942, 944, 946, 948, 950, 952, 954, 956, 958, 960, 962, 964, 966, 968, 970, 972, 974, 975, 976, 977, 978, 979, 981, 982, 983, 985, 987, 989, 991, 993, 994, 995, and 996.

D. Percent Identity

In some embodiments, a salinity and/or oxidative stress tolerance-modulating polypeptide has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to one of the amino acid sequences set forth in SEQ ID NOs: 2, 11, 102, 32, 138, 149, 441, 172, 764, 389, 316, 200, 697, 239, 892, 922, or 693. Polypeptides having such a percent sequence identity often have a domain indicative of a salinity and/or oxidative stress-modulating polypeptide and/or have an HMM bit score that is greater than 15, as discussed above. Examples of amino acid sequences of salinity and/or oxidative stress tolerance-modulating polypeptides having at least 80% sequence identity to one of the amino acid sequences set forth in SEQ ID NOs: 2, 11, 102, 32, 138, 149, 441, 172, 764, 389, 316, 200, 697, 239, 892, 922, or 693 are provided in FIGS. 1-16 and in the sequence listing.

“Percent sequence identity” refers to the degree of sequence identity between any given reference sequence, e.g., SEQ ID NOs: 2, 11, 102, 32, 138, 149, 441, 172, 764, 389, 316, 200, 697, 239, 892, 922, or 693, and a candidate salinity and/or oxidative stress-modulating sequence. A candidate sequence typically has a length that is from 80 percent to 200 percent of the length of the reference sequence, e.g., 82, 85, 87, 89, 90, 93, 95, 97, 99, 100, 105, 110, 115, 120, 130, 140, 150, 160, 170, 180, 190, or 200 percent of the length of the reference sequence. A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). Chema et al., Nucleic Acids Res., 31(13):3497-500 (2003).

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

In some cases, a salinity and/or oxidative stress tolerance-modulating polypeptide has an amino acid sequence with at least 50% sequence identity, e.g., 50%, 52%, 56%, 59%, 61%, 65%, 70%, 75%, 80%, 85%, 90%, 95%, 97%, 98%, or 99% sequence identity, to one or more of the amino acid sequence set forth in SEQ ID NO: 2, 11, 102, 32, 138, 149, 441, 172, 764, 389, 316, 200, 697, 239, 892, 922, or 693 Amino acid sequences of polypeptides having high sequence identity to the polypeptide set forth in SEQ ID NO: 2, 11, 102, 32, 138, 149, 441, 172, 764, 389, 316, 200, 697, 239, 892, 922, or 693 are provided in the Sequence Listing and in FIGS. 1-16.

E. Other Sequences

It should be appreciated that a salinity and/or oxidative stress tolerance-modulating polypeptide can include additional amino acids that are not involved in salinity and/or oxidative stress tolerance modulation, and thus such a polypeptide can be longer than would otherwise be the case. For example, a salinity and/or oxidative stress-tolerance modulating polypeptide can include a purification tag, a chloroplast transit peptide, an amyloplast transit peptide, a mitochondrial transit peptide, or a leader sequence added to the amino or carboxy terminus. In some embodiments, a salinity and/or oxidative stress-tolerance modulating polypeptide includes an amino acid sequence that functions as a reporter, e.g., a green fluorescent protein or yellow fluorescent protein.

### III. NUCLEIC ACIDS

Nucleic acids described herein include nucleic acids that are effective to modulate salinity and/or oxidative stress tolerance levels when transcribed in a plant or plant cell. Such nucleic acids include, without limitation, those that encode a salinity and/or oxidative stress tolerance-modulating polypeptide and those that can be used to inhibit expression of a salinity and/or oxidative stress tolerance-modulating polypeptide via a nucleic acid based method.

A. Nucleic Acids Encoding Salinity and/or Oxidative Stress Tolerance-Modulating Polypeptides

Nucleic acids encoding salinity and/or oxidative stress tolerance-modulating polypeptides are described herein. Examples of such nucleic acids include SEQ ID NOs: 1, 10, 101, 31, 137, 148, 440, 171, 763, 388, 315, 199, 696, 238, 891, 921, or 692, as described in more detail below.

A salinity and/or oxidative stress tolerance-modulating nucleic acid can comprise the nucleotide sequence set forth in SEQ ID NO: 1. Alternatively, a salinity and/or oxidative stress tolerance-modulating nucleic acid can be a variant of the nucleic acid having the nucleotide sequence set forth in SEQ ID NO: 1. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 1. Examples of such nucleotide sequences can be found in the sequence listing and include SEQ ID NO: 3, 5, and 8.

A salinity and/or oxidative stress tolerance-modulating nucleic acid can comprise the nucleotide sequence set forth in SEQ ID NO: 10. Alternatively, a salinity and/or oxidative stress tolerance-modulating nucleic acid can be a variant of the nucleic acid having the nucleotide sequence set forth in SEQ ID NO: 10. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 10. Examples of such nucleotide sequences can be found in the sequence listing and include SEQ ID NO: 12, 14, 16, 18, 20, 22, 24, 26, and 28.

A salinity and/or oxidative stress tolerance-modulating nucleic acid can comprise the nucleotide sequence set forth in SEQ ID NO: 101. Alternatively, a salinity and/or oxidative stress tolerance-modulating nucleic acid can be a variant of the nucleic acid having the nucleotide sequence set forth in SEQ ID NO: 101. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 101. Examples of such nucleotide sequences can be found in the sequence listing and include SEQ ID NO: 105, 107, 109, 111, 113, 121, 123, 126, 131, 133, and 135.

A salinity and/or oxidative stress tolerance-modulating nucleic acid can comprise the nucleotide sequence set forth in SEQ ID NO: 31. Alternatively, a salinity and/or oxidative stress tolerance-modulating nucleic acid can be a variant of the nucleic acid having the nucleotide sequence set forth in SEQ ID NO: 31. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 31. Examples of such nucleotide sequences can be found in the sequence listing and include SEQ ID NO: 34, 36, 39, 41, 43, 45, 47, 50, 52, 55, 57, 59, 61, 70, 72, 74, 76, 78, 80, 82, 84, 86, 88, 90, 92, 94, and 96.

A salinity and/or oxidative stress tolerance-modulating nucleic acid can comprise the nucleotide sequence set forth in SEQ ID NO: 137. Alternatively, a salinity and/or oxidative stress tolerance-modulating nucleic acid can be a variant of the nucleic acid having the nucleotide sequence set forth in SEQ ID NO: 137. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 137. Examples of such nucleotide sequences can be found in the sequence listing and include SEQ ID NO: 137, 139, 141, 143, and 145.

A salinity and/or oxidative stress tolerance-modulating nucleic acid can comprise the nucleotide sequence set forth in SEQ ID NO: 148. Alternatively, a salinity and/or oxidative stress tolerance-modulating nucleic acid can be a variant of the nucleic acid having the nucleotide sequence set forth in SEQ ID NO: 148. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 148. Examples of such nucleotide sequences can be found in the sequence listing and include SEQ ID NO: 148, 150, 152, 156, 158, 160, 166, and 168.

A salinity and/or oxidative stress tolerance-modulating nucleic acid can comprise the nucleotide sequence set forth in SEQ ID NO: 440. Alternatively, a salinity and/or oxidative stress tolerance-modulating nucleic acid can be a variant of the nucleic acid having the nucleotide sequence set forth in SEQ ID NO: 440. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 440. Examples of such nucleotide sequences can be found in the sequence listing and include SEQ ID NO: 443, 448, 455, 466, 476, 478, 483, 485, 488, 495, 497, 499, 501, 510, 515, 521, 526, 528, 530, 535, 542, 545, 548, 554, 558, 561, 568, 570, 573, 575, 579, 581, 584, 587, 589, 596, 599, 602, 604, 607, 612, 619, 621, 623, 628, 630, 632, 634, 636, 638, 645, 647, 649, 652, 656, 658, 667, 670, 672, 674, 676, 679, 681, 683, 685, 687, and 689.

A salinity and/or oxidative stress tolerance-modulating nucleic acid can comprise the nucleotide sequence set forth in SEQ ID NO: 171. Alternatively, a salinity and/or oxidative stress tolerance-modulating nucleic acid can be a variant of the nucleic acid having the nucleotide sequence set forth in SEQ ID NO: 171. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 171. Examples of such nucleotide sequences can be found in the sequence listing and include SEQ ID NO: 181, 183, 185, 190, 193, 195, and 197.

A salinity and/or oxidative stress tolerance-modulating nucleic acid can comprise the nucleotide sequence set forth in SEQ ID NO: 763. Alternatively, a salinity and/or oxidative stress tolerance-modulating nucleic acid can be a variant of the nucleic acid having the nucleotide sequence set forth in SEQ ID NO: 763. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 763. Examples of such nucleotide sequences can be found in the sequence listing and include SEQ ID NO: 768, 772, 778, 783, 787, 789, 791, 794, 799, 800, 803, 807, 811, 821, 823, 828, 830, 833, 835, 837, 839, 842, 844, 853, 859, 873, 875, 877, 879, 881, 883, 885, 887, and 889.

A salinity and/or oxidative stress tolerance-modulating nucleic acid can comprise the nucleotide sequence set forth in SEQ ID NO: 388. Alternatively, a salinity and/or oxidative stress tolerance-modulating nucleic acid can be a variant of the nucleic acid having the nucleotide sequence set forth in SEQ ID NO: 388. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 388. Examples of such nucleotide sequences can be found in the sequence listing and include SEQ ID NO: 390, 391, 393, 395, 397, 399, 404, 409, 413, 415, 420, 423, 425, 427, 429, and 435.

A salinity and/or oxidative stress tolerance-modulating nucleic acid can comprise the nucleotide sequence set forth in SEQ ID NO: 315. Alternatively, a salinity and/or oxidative stress tolerance-modulating nucleic acid can be a variant of the nucleic acid having the nucleotide sequence set forth in SEQ ID NO: 315. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 315. Examples of such nucleotide sequences can be found in the sequence listing and include SEQ ID NO: 317, 322, 330, 332, 334, 336, 338, 343, 345, 347, 349, 352, 354, 358, 360, 362, 364, 366, 368, 376, 379, 381, 383, and 385.

A salinity and/or oxidative stress tolerance-modulating nucleic acid can comprise the nucleotide sequence set forth in SEQ ID NO: 199. Alternatively, a salinity and/or oxidative stress tolerance-modulating nucleic acid can be a variant of the nucleic acid having the nucleotide sequence set forth in SEQ ID NO: 199. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 199. Examples of such nucleotide sequences can be found in the sequence listing and include SEQ ID NO: 207, 205, 203, 234, 236, 216, 210, 218, 231, 201, 229, 222, 227, and 224.

A salinity and/or oxidative stress tolerance-modulating nucleic acid can comprise the nucleotide sequence set forth in SEQ ID NO: 696. Alternatively, a salinity and/or oxidative stress tolerance-modulating nucleic acid can be a variant of the nucleic acid having the nucleotide sequence set forth in SEQ ID NO: 696. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 696. Examples of such nucleotide sequences can be found in the sequence listing and include SEQ ID NO: 734, 748, 738, 731, 710, 736, 729, 760, 756, 758, 754, 717, 698, 713, 727, 744, 702, 750, 725, 721, 719, and 708.

A salinity and/or oxidative stress tolerance-modulating nucleic acid can comprise the nucleotide sequence set forth in SEQ ID NO: 238. Alternatively, a salinity and/or oxidative stress tolerance-modulating nucleic acid can be a variant of the nucleic acid having the nucleotide sequence set forth in SEQ ID NO: 238. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 238. Examples of such nucleotide sequences can be found in the sequence listing and include SEQ ID NO: 283, 307, 269, 256, 260, 278, 248, 252, 254, 296, 242, 274, 244, 280, 285, 300, 303, 305, 298, 240, 263, 258, 287, 289, 265, and 246.

A salinity and/or oxidative stress tolerance-modulating nucleic acid can comprise the nucleotide sequence set forth in SEQ ID NO: 891. Alternatively, a salinity and/or oxidative stress tolerance-modulating nucleic acid can be a variant of the nucleic acid having the nucleotide sequence set forth in SEQ ID NO: 891. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 891. Examples of such nucleotide sequences can be found in the sequence listing and include SEQ ID NO: 893, 895, 897, 900, 902, 904, 908, 910, 912, 917, and 919.

A salinity and/or oxidative stress tolerance-modulating nucleic acid can comprise the nucleotide sequence set forth in SEQ ID NO: 921. Alternatively, a salinity and/or oxidative stress tolerance-modulating nucleic acid can be a variant of the nucleic acid having the nucleotide sequence set forth in SEQ ID NO: 921. For example, a salinity and/or oxidative stress tolerance-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO: 921. Examples of such nucleotide sequences can be found in the sequence listing and include SEQ ID NO: 830, 923, 925, 927, 929, 931, 933, 935, 937, 939, 941, 943, 945, 947, 949, 951, 953, 955, 957, 959, 961, 963, 965, 967, 969, 971, 973, 980, 984, 986, 988, 990, and 992.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

B. Use of Nucleic Acids to Modulate Expression of Polypeptides

i. Expression of a Salinity and/or Oxidative Stress Tolerance-Modulating Polypeptide

A nucleic acid encoding one of the salinity and/or oxidative stress tolerance-modulating polypeptides described herein can be used to express the polypeptide in a plant species of interest, typically by transforming a plant cell with a nucleic acid having the coding sequence for the polypeptide operably linked in sense orientation to one or more regulatory regions. It will be appreciated that because of the degeneracy of the genetic code, a number of nucleic acids can encode a particular salinity and/or oxidative stress tolerance-modulating polypeptide; i.e., for many amino acids, there is more than one nucleotide triplet that serves as the codon for the amino acid. Thus, codons in the coding sequence for a given salinity and/or oxidative stress tolerance-modulating polypeptide can be modified such that optimal expression in a particular plant species is obtained, using appropriate codon bias tables for that species.

In some cases, expression of a salinity and/or oxidative stress tolerance-modulating polypeptide inhibits one or more functions of an endogenous polypeptide. For example, a nucleic acid that encodes a dominant negative polypeptide can be used to inhibit protein function. A dominant negative polypeptide typically is mutated or truncated relative to an endogenous wild type polypeptide, and its presence in a cell inhibits one or more functions of the wild type polypeptide in that cell, i.e., the dominant negative polypeptide is genetically dominant and confers a loss of function. The mechanism by which a dominant negative polypeptide confers such a phenotype can vary but often involves a protein-protein interaction or a protein-DNA interaction. For example, a dominant negative polypeptide can be an enzyme that is truncated relative to a native wild type enzyme, such that the truncated polypeptide retains domains involved in binding a first protein but lacks domains involved in binding a second protein. The truncated polypeptide is thus unable to properly modulate the activity of the second protein. See, e.g., US 2007/0056058. As another example, a point mutation that results in a non-conservative amino acid substitution in a catalytic domain can result in a dominant negative polypeptide. See, e.g., US 2005/032221. As another example, a dominant negative polypeptide can be a transcription factor that is truncated relative to a native wild type transcription factor, such that the truncated polypeptide retains the DNA binding domain(s) but lacks the activation domain(s). Such a truncated polypeptide can inhibit the wild type transcription factor from binding DNA, thereby inhibiting transcription activation.

ii Inhibition of Expression of a salinity and/or oxidative stress tolerance-Modulating Polypeptide

Polynucleotides and recombinant constructs described herein can be used to inhibit expression of a salinity and/or oxidative stress tolerance-modulating polypeptide in a plant species of interest. See, e.g., Matzke and Birchler, Nature Reviews Genetics 6:24-35 (2005); Akashi et al., Nature Reviews Mol. Cell Biology 6:413-422 (2005); Mittal, Nature Reviews Genetics 5:355-365 (2004); Dorsett and Tuschl, Nature Reviews Drug Discovery 3: 318-329 (2004); and Nature Reviews RNA interference collection, October 2005 at nature.com/reviews/focus/mai. Typically, at least a fragment of a nucleic acid encoding salinity and/or oxidative stress tolerance-modulating polypeptides and/or its complement is expressed. A fragment is typically at least 20 nucleotides long, as needed for the methods noted below. A number of nucleic acid based methods, including antisense RNA, ribozyme directed RNA cleavage, post-transcriptional gene silencing (PTGS), e.g., RNA interference (RNAi), and transcriptional gene silencing (TGS) are known to inhibit gene expression in plants. Antisense technology is one well-known method. In this method, a nucleic acid segment from a gene to be repressed is cloned and operably linked to a regulatory region and a transcription termination sequence so that the antisense strand of RNA is transcribed. The recombinant construct is then transformed into plants, as described herein, and the antisense strand of RNA is produced. The nucleic acid segment need not be the entire sequence of the gene to be repressed, but typically will be substantially complementary to at least a portion of the sense strand of the gene to be repressed. Generally, higher homology can be used to compensate for the use of a shorter sequence. Typically, a sequence of at least 30 nucleotides is used, e.g., at least 40, 50, 80, 100, 200, 500 nucleotides or more.

In another method, a nucleic acid can be transcribed into a ribozyme, or catalytic RNA, that affects expression of an mRNA. See, U.S. Pat. No. 6,423,885. Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contains a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants”, Edited by Turner, P. C., Humana Press Inc., Totowa, N.J. RNA endoribonucleases which have been described, such as the one that occurs naturally in Tetrahymena thermophila, can be useful. See, for example, U.S. Pat. Nos. 4,987,071 and 6,423,885.

PTGS, e.g., RNAi, can also be used to inhibit the expression of a gene. For example, a construct can be prepared that includes a sequence that is transcribed into an RNA that can anneal to itself, e.g., a double stranded RNA having a stem-loop structure. In some embodiments, one strand of the stem portion of a double stranded RNA comprises a sequence that is similar or identical to the sense coding sequence of a salinity and/or oxidative stress tolerance-modulating polypeptide, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand of the coding sequence of the salinity and/or oxidative stress tolerance-modulating polypeptide, and can have a length that is shorter, the same as, or longer than the corresponding length of the sense sequence. In some cases, one strand of the stem portion of a double stranded RNA comprises a sequence that is similar or identical to the 3′ or 5′ untranslated region of an mRNA encoding a salinity and/or oxidative stress tolerance-modulating polypeptide, and the other strand of the stem portion of the double stranded RNA comprises a sequence that is similar or identical to the sequence that is complementary to the 3′ or 5′ untranslated region, respectively, of the mRNA encoding the salinity and/or oxidative stress tolerance-modulating polypeptide. In other embodiments, one strand of the stem portion of a double stranded RNA comprises a sequence that is similar or identical to the sequence of an intron in the pre-mRNA encoding a salinity and/or oxidative stress tolerance-modulating polypeptide, and the other strand of the stem portion comprises a sequence that is similar or identical to the sequence that is complementary to the sequence of the intron in the pre-mRNA. The loop portion of a double stranded RNA can be from 3 nucleotides to 5,000 nucleotides, e.g., from 3 nucleotides to 25 nucleotides, from 15 nucleotides to 1,000 nucleotides, from 20 nucleotides to 500 nucleotides, or from 25 nucleotides to 200 nucleotides. The loop portion of the RNA can include an intron. A double stranded RNA can have zero, one, two, three, four, five, six, seven, eight, nine, ten, or more stem-loop structures. A construct including a sequence that is operably linked to a regulatory region and a transcription termination sequence, and that is transcribed into an RNA that can form a double stranded RNA, is transformed into plants as described herein. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330, and 20030180945.

Constructs containing regulatory regions operably linked to nucleic acid molecules in sense orientation can also be used to inhibit the expression of a gene. The transcription product can be similar or identical to the sense coding sequence of a salinity and/or oxidative stress tolerance-modulating polypeptide. The transcription product can also be unpolyadenylated, lack a 5′ cap structure, or contain an unspliceable intron. Methods of inhibiting gene expression using a full-length cDNA as well as a partial cDNA sequence are known in the art. See, e.g., U.S. Pat. No. 5,231,020.

In some embodiments, a construct containing a nucleic acid having at least one strand that is a template for both sense and antisense sequences that are complementary to each other is used to inhibit the expression of a gene. The sense and antisense sequences can be part of a larger nucleic acid molecule or can be part of separate nucleic acid molecules having sequences that are not complementary. The sense or antisense sequence can be a sequence that is identical or complementary to the sequence of an mRNA, the 3′ or 5′ untranslated region of an mRNA, or an intron in a pre-mRNA encoding a salinity and/or oxidative stress tolerance-modulating polypeptide. In some embodiments, the sense or antisense sequence is identical or complementary to a sequence of the regulatory region that drives transcription of the gene encoding a salinity and/or oxidative stress tolerance-modulating polypeptide. In each case, the sense sequence is the sequence that is complementary to the antisense sequence.

The sense and antisense sequences can be any length greater than about 12 nucleotides (e.g., 13, 14, 15, 16, 17, 18, 19, 20, 21, 22, 23, 24, 25, 26, 27, 28, 29, 30, or more nucleotides). For example, an antisense sequence can be 21 or 22 nucleotides in length. Typically, the sense and antisense sequences range in length from about 15 nucleotides to about 30 nucleotides, e.g., from about 18 nucleotides to about 28 nucleotides, or from about 21 nucleotides to about 25 nucleotides.

In some embodiments, an antisense sequence is a sequence complementary to an mRNA sequence encoding a salinity and/or oxidative stress tolerance-modulating polypeptide described herein. The sense sequence complementary to the antisense sequence can be a sequence present within the mRNA of the salinity and/or oxidative stress tolerance-modulating polypeptide. Typically, sense and antisense sequences are designed to correspond to a 15-30 nucleotide sequence of a target mRNA such that the level of that target mRNA is reduced.

In some embodiments, a construct containing a nucleic acid having at least one strand that is a template for more than one sense sequence (e.g., 2, 3, 4, 5, 6, 7, 8, 9, 10 or more sense sequences) can be used to inhibit the expression of a gene. Likewise, a construct containing a nucleic acid having at least one strand that is a template for more than one antisense sequence (e.g., 2, 3, 4, 5, 6, 7, 8, 9, 10 or more antisense sequences) can be used to inhibit the expression of a gene. For example, a construct can contain a nucleic acid having at least one strand that is a template for two sense sequences and two antisense sequences. The multiple sense sequences can be identical or different, and the multiple antisense sequences can be identical or different. For example, a construct can have a nucleic acid having one strand that is a template for two identical sense sequences and two identical antisense sequences that are complementary to the two identical sense sequences. Alternatively, an isolated nucleic acid can have one strand that is a template for (1) two identical sense sequences 20 nucleotides in length, (2) one antisense sequence that is complementary to the two identical sense sequences 20 nucleotides in length, (3) a sense sequence 30 nucleotides in length, and (4) three identical antisense sequences that are complementary to the sense sequence 30 nucleotides in length. The constructs provided herein can be designed to have any arrangement of sense and antisense sequences. For example, two identical sense sequences can be followed by two identical antisense sequences or can be positioned between two identical antisense sequences.

A nucleic acid having at least one strand that is a template for one or more sense and/or antisense sequences can be operably linked to a regulatory region to drive transcription of an RNA molecule containing the sense and/or antisense sequence(s). In addition, such a nucleic acid can be operably linked to a transcription terminator sequence, such as the terminator of the nopaline synthase (nos) gene. In some cases, two regulatory regions can direct transcription of two transcripts: one from the top strand, and one from the bottom strand. See, for example, Yan et al., Plant Physiol., 141:1508-1518 (2006). The two regulatory regions can be the same or different. The two transcripts can form double-stranded RNA molecules that induce degradation of the target RNA. In some cases, a nucleic acid can be positioned within a T-DNA or plant-derived transfer DNA (P-DNA) such that the left and right T-DNA border sequences, or the left and right border-like sequences of the P-DNA, flank or are on either side of the nucleic acid. See, US 2006/0265788. The nucleic acid sequence between the two regulatory regions can be from about 15 to about 300 nucleotides in length. In some embodiments, the nucleic acid sequence between the two regulatory regions is from about 15 to about 200 nucleotides in length, from about 15 to about 100 nucleotides in length, from about 15 to about 50 nucleotides in length, from about 18 to about 50 nucleotides in length, from about 18 to about 40 nucleotides in length, from about 18 to about 30 nucleotides in length, or from about 18 to about 25 nucleotides in length.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Summerton and Weller, 1997, Antisense Nucleic Acid Drug Dev., 7:187-195; Hyrup et al., Bioorgan. Med. Chem., 4:5-23 (1996). In addition, the deoxyphosphate backbone can be replaced with, for example, a phosphorothioate or phosphorodithioate backbone, a phosphoroamidite, or an alkyl phosphotriester backbone.

C. Constructs/Vectors

Recombinant constructs provided herein can be used to transform plants or plant cells in order to modulate salinity and/or oxidative stress tolerance levels. A recombinant nucleic acid construct can comprise a nucleic acid encoding a salinity and/or oxidative stress tolerance-modulating polypeptide as described herein, operably linked to a regulatory region suitable for expressing the salinity and/or oxidative stress tolerance-modulating polypeptide in the plant or cell. Thus, a nucleic acid can comprise a coding sequence that encodes any of the salinity and/or oxidative stress tolerance-modulating polypeptides as set forth in SEQ ID NOs: 2, 11, 102, 32, 138, 149, 441, 172, 764, 389, 316, 200, 697, 239, 892, 922, 693 or homologs thereof, including, but not limited to, any of the amino acid sequences set forth in the sequence listing. Examples of nucleic acids encoding salinity and/or oxidative stress tolerance-modulating polypeptides are set forth in SEQ ID NOs: 1, 3, 5, 8, 10, 12, 14, 16, 18, 20, 22, 24, 26, 28, 31, 34, 36, 39, 41, 43, 45, 47, 50, 52, 55, 7, 59, 61, 70, 72, 74, 76, 78, 80, 82, 84, 86, 88, 90, 92, 94, 96, 101, 105, 107, 109, 111, 113, 121, 123, 126, 131, 133, 135, 137, 139, 141, 145, 148, 150, 152, 156, 158, 160, 166, 168, 170, 171, 173, 177, 181, 183, 185, 190, 193, 195, 197, 199, 201, 203, 205, 207, 210, 216, 218, 222, 224, 227, 229, 231, 234, 236, 238, 240, 242, 244, 246, 248, 252, 254, 256, 258, 260, 263, 265, 269, 274, 278, 280, 283, 285, 287, 289, 296, 298, 300, 303, 305, 307, 315, 317, 322, 330, 332, 334, 336, 338, 343, 345, 347, 349, 352, 354, 358, 360, 362, 364, 366, 368, 376, 379, 381, 383, 385, 387, 388, 391, 393, 395, 397, 399, 404, 409, 413, 415, 420, 423, 425, 427, 429, 435, 439, 440, 443, 448, 455, 466, 476, 478, 483, 485, 488, 495, 497, 499, 501, 510, 515, 521, 526, 528, 530, 535, 542, 545, 548, 554, 558, 561, 568, 570, 573, 575, 579, 581, 584, 587, 589, 596, 599, 602, 604, 607, 612, 619, 621, 623, 628, 630, 632, 634, 636, 638, 645, 647, 649, 652, 656, 658, 667, 670, 672, 674, 676, 679, 681, 683, 685, 687, 689, 691, 692, 695, 696, 698, 702, 708, 710, 713, 717, 719, 721, 725, 727, 729, 731, 734, 736, 738, 744, 748, 750, 754, 756, 758, 760, 762, 763, 768, 772, 778, 783, 787, 789, 791, 794, 799, 800, 803, 807, 811, 821, 823, 828, 830, 833, 835, 837, 839, 842, 844, 853, 859, 865, 873, 875, 877, 879, 881, 883, 885, 887, 889, 891, 893, 895, 897, 900, 902, 904, 908, 910, 912, 917, 919, 921, 923, 925, 927, 929, 931, 933, 935, 937, 939, 941, 943, 945, 947, 949, 951, 953, 955, 957, 959, 961, 963, 965, 967, 969, 971, 973, 980, 984, 986, 988, 990, or 992.

The salinity and/or oxidative stress tolerance-modulating polypeptide encoded by a recombinant nucleic acid can be a native salinity and/or oxidative stress tolerance-modulating polypeptide, or can be heterologous to the cell. In some cases, the recombinant construct contains a nucleic acid that inhibits expression of a salinity and/or oxidative stress tolerance-modulating polypeptide, operably linked to a regulatory region. Examples of suitable regulatory regions are described in the section entitled “Regulatory Regions.”

Vectors containing recombinant nucleic acid constructs such as those described herein also are provided. Suitable vector backbones include, for example, those routinely used in the art such as plasmids, viruses, artificial chromosomes, BACs, YACs, or PACs. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a plant cell. For example, a marker can confer biocide resistance, such as resistance to an antibiotic (e.g., kanamycin, G418, bleomycin, or hygromycin), or an herbicide (e.g., glyphosate, chlorsulfuron or phosphinothricin). In addition, an expression vector can include a tag sequence designed to facilitate manipulation or detection (e.g., purification or localization) of the expressed polypeptide. Tag sequences, such as luciferase, β-glucuronidase (GUS), green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

D. Regulatory Regions

The choice of regulatory regions to be included in a recombinant construct depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning regulatory regions relative to the coding sequence. Transcription of a nucleic acid can be modulated in a similar manner.

Some suitable promoters initiate transcription only, or predominantly, in certain cell types. The choice of regulatory regions to be included in a recombinant construct depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning regulatory regions relative to the coding sequence. Transcription of a nucleic acid can be modulated in a similar manner

Some suitable regulatory regions initiate transcription only, or predominantly, in certain cell types. Methods for identifying and characterizing regulatory regions in plant genomic DNA are known, including, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996).

Examples of various classes of regulatory regions are described below. Some of the regulatory regions indicated below as well as additional regulatory regions are described in more detail in U.S. Patent Application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 11/408,791; 11/414,142; 10/950,321; 11/360,017; PCT/US05/011105; PCT/US05/23639; PCT/US05/034308; PCT/US05/034343; and PCT/US06/038236; PCT/US06/040572; and PCT/US07/62762, each of which is hereby expressly incorporated by reference in its entirety.

Additional examples of regulatory regions are described in U.S. Application Ser. No. 61/025,697, which is hereby expressly incorporated by reference in its entirety.

For example, the sequences of regulatory regions p326, YP0144, YP0190, p13879, YP0050, p32449, 21876, YP0158, YP0214, YP0380, PT0848, PT0633, YP0128, YP0275, PT0660, PT0683, PT0758, PT0613, PT0672, PT0688, PT0837, YP0092, PT0676, PT0708, YP0396, YP0007, YP0111, YP0103, YP0028, YP0121, YP0008, YP0039, YP0115, YP0119, YP0120, YP0374, YP0101, YP0102, YP0110, YP0117, YP0137, YP0285, YP0212, YP0097, YP0107, YP0088, YP0143, YP0156, PT0650, PT0695, PT0723, PT0838, PT0879, PT0740, PT0535, PT0668, PT0886, PT0585, YP0381, YP0337, PT0710, YP0356, YP0385, YP0384, YP0286, YP0377, PD1367, PT0863, PT0829, PT0665, PT0678, YP0086, YP0188, YP0263, PT0743 and YP0096 are set forth in the sequence listing of PCT/US06/040572; the sequence of regulatory region PT0625 is set forth in the sequence listing of PCT/US05/034343; the sequences of regulatory regions PT0623, YP0388, YP0087, YP0093, YP0108, YP0022 and YP0080 are set forth in the sequence listing of U.S. patent application Ser. No. 11/172,703; the sequence of regulatory region PRO924 is set forth in the sequence listing of PCT/US07/62762; and the sequences of regulatory regions p530c10, pOsFIE2-2, pOsMEA, pOsYp102, and pOsYp285 are set forth in the sequence listing of PCT/US06/038236.

It will be appreciated that a regulatory region may meet criteria for one classification based on its activity in one plant species, and yet meet criteria for a different classification based on its activity in another plant species.

i. Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326, YP0144, YP0190, p13879, YP0050, p32449, 21876, YP0158, YP0214, YP0380, PT0848, and PT0633 promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

ii. Root Promoters

Root-active promoters confer transcription in root tissue, e.g., root endodermis, root epidermis, or root vascular tissues. In some embodiments, root-active promoters are root-preferential promoters, i.e., confer transcription only or predominantly in root tissue. Root-preferential promoters include the YP0128, YP0275, PT0625, PT0660, PT0683, and PT0758 promoters. Other root-preferential promoters include the PT0613, PT0672, PT0688, and PT0837 promoters, which drive transcription primarily in root tissue and to a lesser extent in ovules and/or seeds. Other examples of root-preferential promoters include the root-specific subdomains of the CaMV 35S promoter (Lam et al., Proc. Natl. Acad. Sci. USA, 86:7890-7894 (1989)), root cell specific promoters reported by Conkling et al., Plant Physiol., 93:1203-1211 (1990), and the tobacco RD2 promoter.

iii. Maturing Endosperm Promoters

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin promoter (Bustos et al., Plant Cell, 1(9):839-853 (1989)), the soybean trypsin inhibitor promoter (Riggs et al., Plant Cell, 1(6):609-621 (1989)), the ACP promoter (Baerson et al., Plant Mol. Biol., 22(2):255-267 (1993)), the stearoyl-ACP desaturase promoter (Slocombe et al., Plant Physiol., 104(4):167-176 (1994)), the soybean α′ subunit of β-conglycinin promoter (Chen et al., Proc. Natl. Acad. Sci. USA, 83:8560-8564 (1986)), the oleosin promoter (Hong et al., Plant Mol. Biol., 34(3):549-555 (1997)), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell Biol., 13:5829-5842 (1993)), the beta-amylase promoter, and the barley hordein promoter. Other maturing endosperm promoters include the YP0092, PT0676, and PT0708 promoters.

iv. Ovary Tissue Promoters

Promoters that are active in ovary tissues such as the ovule wall and mesocarp can also be useful, e.g., a polygalacturonidase promoter, the banana TRX promoter, the melon actin promoter, YP0396, and PT0623. Examples of promoters that are active primarily in ovules include YP0007, YP0111, YP0092, YP0103, YP0028, YP0121, YP0008, YP0039, YP0115, YP0119, YP0120, and YP0374.

v. Embryo Sac/Early Endosperm Promoters

To achieve expression in embryo sac/early endosperm, regulatory regions can be used that are active in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, GenBank No. U93215); Arabidopsis atmycl (see, Urao (1996) Plant Mol. Biol., 32:571-57; Conceicao (1994) Plant, 5:493-505); Arabidopsis FIE (GenBank No. AF129516); Arabidopsis MEA; Arabidopsis FIS2 (GenBank No. AF096096); and FIE 1.1 (U.S. Pat. No. 6,906,244). Other promoters that may be suitable include those derived from the following genes: maize MAC1 (see, Sheridan (1996) Genetics, 142:1009-1020); maize Cat3 (see, GenBank No. L05934; Abler (1993) Plant Mol. Biol., 22:10131-1038). Other promoters include the following Arabidopsis promoters: YP0039, YP0101, YP0102, YP0110, YP0117, YP0119, YP0137, DME, YP0285, and YP0212. Other promoters that may be useful include the following rice promoters: p530c10, pOsFIE2-2, pOsMEA, pOsYp102, and pOsYp285.

vi. Embryo Promoters

Regulatory regions that preferentially drive transcription in zygotic cells following fertilization can provide embryo-preferential expression. Most suitable are promoters that preferentially drive transcription in early stage embryos prior to the heart stage, but expression in late stage and maturing embryos is also suitable. Embryo-preferential promoters include the barley lipid transfer protein (Ltp1) promoter (Plant Cell Rep (2001) 20:647-654), YP0097, YP0107, YP0088, YP0143, YP0156, PT0650, PT0695, PT0723, PT0838, PT0879, and PT0740.

vii. Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcbl*2 promoter (Cerdan et al., Plant Mol. Biol., 33:245-255 (1997)), the Arabidopsis thaliana SUC2 sucrose-H+ symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535, PT0668, PT0886, YP0144, YP0380 and PT0585.

viii. Vascular Tissue Promoters

Examples of promoters that have high or preferential activity in vascular bundles include YP0087, YP0093, YP0108, YP0022, and YP0080. Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

ix. Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380, PT0848, YP0381, YP0337, PT0633, YP0374, PT0710, YP0356, YP0385, YP0396, YP0388, YP0384, PT0688, YP0286, YP0377, PD1367, and PD0901. Examples of nitrogen-inducible promoters include PT0863, PT0829, PT0665, and PT0886. Examples of shade-inducible promoters include PRO924 and PT0678. An example of a promoter induced by salt is rd29A (Kasuga et al. (1999) Nature Biotech 17: 287-291).

x. Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

xi. Stem Promoters

A stem promoter may be specific to one or more stem tissues or specific to stem and other plant parts. Stem promoters may have high or preferential activity in, for example, epidermis and cortex, vascular cambium, procambium, or xylem. Examples of stem promoters include YP0018 which is disclosed in US20060015970 and CryIA(b) and CryIA(c) (Braga et al., Journal of New Seeds 5:209-221 (2003)).

xii. Other Promoters

Other classes of promoters include, but are not limited to, shoot-preferential, callus-preferential, trichome cell-preferential, guard cell-preferential such as PT0678, tuber-preferential, parenchyma cell-preferential, and senescence-preferential promoters. Promoters designated YP0086, YP0188, YP0263, PT0758, PT0743, PT0829, YP0119, and YP0096, as described in the above-referenced patent applications, may also be useful.

xiii. Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, for example, more than one regulatory region can be operably linked to the sequence of a polynucleotide encoding a salt and/or oxidative stress tolerance modulating polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

Alternatively, misexpression can be accomplished using a two component system, whereby the first component consists of a transgenic plant comprising a transcriptional activator operatively linked to a promoter and the second component consists of a transgenic plant that comprise a nucleic acid molecule of the invention operatively linked to the target-binding sequence/region of the transcriptional activator. The two transgenic plants are crossed and the nucleic acid molecule of the invention is expressed in the progeny of the plant. In another alternative embodiment of the present invention, the misexpression can be accomplished by having the sequences of the two component system transformed in one transgenic plant line.

In some embodiments, a regulatory region may comprise Ceres Promoter PD2995/Ceres Promoter PD2263 (SEQ ID NO: 390), Ceres Promoter PD2999/Ceres Promoter PD2258 (SEQ ID NO: 700), Ceres Promoter PD3006/Ceres Promoter YP0050 (SEQ ID NO: 715), Ceres Promoter PD3141 (SEQ ID NO: 701), Ceres Promoter PD3147/Ceres Promoter YP2663 (SEQ ID NO: 705), Ceres Promoter PT0633 (SEQ ID NO: 143), Ceres Promoter PT0746 (SEQ ID NO: 212), Ceres Promoter PT0758 (SEQ ID NO: 176), Ceres Promoter PT0822 (SEQ ID NO: 716), Ceres Promoter PT0839 (SEQ ID NO: 373), Ceres Promoter PT0888 (SEQ ID NO: 209), Ceres Promoter PT0960 (SEQ ID NO: 213), Ceres Promoter PT0998 (SEQ ID NO: 220), Ceres Promoter PT1026 (SEQ ID NO: 221), Ceres Promoter YP0286 (SEQ ID NO: 268), Ceres Promoter YP0337 (SEQ ID NO: 276), Ceres Promoter YP0381 (SEQ ID NO: 277), Ceres Promoter YP0388 (SEQ ID NO: 309), Ceres Promoter YP1692 (SEQ ID NO: 310), Ceres Promoter YP1894 (SEQ ID NO: 311), Ceres Promoter YP1976 (SEQ ID NO: 312), Ceres Promoter YP2016 (SEQ ID NO: 313), Ceres Promoter YP2097 (SEQ ID NO: 314), Ceres Promoter YP2219 (SEQ ID NO: 267), Ceres Promoter YP2538 (SEQ ID NO: 325), Ceres Promoter YP2552 (SEQ ID NO: 326), Ceres Promoter YP2563 (SEQ ID NO: 327), Ceres Promoter YP2571 (SEQ ID NO: 328), Ceres Promoter YP2573 (SEQ ID NO: 215), Ceres Promoter YP2585 (SEQ ID NO: 214), Ceres Promoter YP2590 (SEQ ID NO: 329), Ceres Promoter YP2606 (SEQ ID NO: 341), Ceres Promoter YP2608 (SEQ ID NO: 342), Ceres Promoter YP2680 (SEQ ID NO: 704), Ceres Promoter YP2683 (SEQ ID NO: 370), Ceres Promoter YP2816 (SEQ ID NO: 371), or Ceres Promoter YP2832 (SEQ ID NO: 372).

### IV. TRANSGENIC PLANTS AND PLANT CELLS

### A. Transformation

The invention also features transgenic plant cells and plants comprising at least one recombinant nucleic acid construct described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. As used herein, a transgenic plant also refers to progeny of an initial transgenic plant, as long as the progeny inherits the transgene. Seeds produced by a transgenic plant can be grown and then selfed (or outcrossed and selfed) to obtain seeds homozygous for the nucleic acid construct.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium. A solid medium can be, for example, Murashige and Skoog (MS) medium containing agar and a suitable concentration of an auxin, e.g., 2,4-dichlorophenoxyacetic acid (2,4-D), and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous salinity and/or oxidative stress tolerance-modulating polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880; 5,204,253; 6,329,571 and 6,013,863. If a cell or cultured tissue is used as the recipient tissue for transformation, plants can be regenerated from transformed cultures if desired, by techniques known to those skilled in the art.

### B. Screening/Selection

A population of transgenic plants can be screened and/or selected for those members of the population that have a trait or phenotype conferred by expression of the transgene. For example, a population of progeny of a single transformation event can be screened for those plants having a desired level of expression of a salinity and/or oxidative stress tolerance-modulating polypeptide or nucleic acid. Physical and biochemical methods can be used to identify expression levels. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known. As an alternative, a population of plants comprising independent transformation events can be screened for those plants having a desired trait, such as a modulated level of salinity and/or oxidative stress tolerance. Selection and/or screening can be carried out over one or more generations, and/or in more than one geographic location. In some cases, transgenic plants can be grown and selected under conditions which induce a desired phenotype or are otherwise necessary to produce a desired phenotype in a transgenic plant. In addition, selection and/or screening can be applied during a particular developmental stage in which the phenotype is expected to be exhibited by the plant. Selection and/or screening can be carried out to choose those transgenic plants having a statistically significant difference in a salinity and/or oxidative stress tolerance level relative to a control plant that lacks the transgene. Selected or screened transgenic plants have an altered phenotype as compared to a corresponding control plant, as described in the “Transgenic Plant Phenotypes” section herein.

A population of transgenic plants can be screened and/or selected for those members of the population that have a trait or phenotype conferred by expression of the transgene. For example, a population of progeny of a single transformation event can be screened for those plants having a desired level of expression of a saline and/or oxidative stress tolerance-modulating polypeptide and/or nucleic acid. Physical and biochemical methods can be used to identify expression levels. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known. As an alternative, a population of plants comprising independent transformation events can be screened for those plants having a desired trait, such as a modulated level of saline and/or oxidative stress tolerance. Selection and/or screening can be carried out over one or more generations, and/or in more than one geographic location. In some cases, transgenic plants can be grown and selected under conditions which induce a desired phenotype or are otherwise necessary to produce a desired phenotype in a transgenic plant. In addition, selection and/or screening can be applied during a particular developmental stage in which the phenotype is expected to be exhibited by the plant. Selection and/or screening can be carried out to choose those transgenic plants having a statistically significant difference in a saline and/or oxidative stress tolerance level relative to a control plant that lacks the transgene. Selected or screened transgenic plants have an altered phenotype as compared to a corresponding control plant, as described in the “Transgenic Plant Phenotypes” section herein.

C. Plant Species

The polynucleotides and vectors described herein can be used to transform a number of monocotyledonous and dicotyledonous plants and plant cell systems, including species from one of the following families: Acanthaceae, Alliaceae, Alstroemeriaceae, Amaryllidaceae, Apocynaceae, Arecaceae, Asteraceae, Berberidaceae, Bixaceae, Brassicaceae, Bromeliaceae, Cannabaceae, Caryophyllaceae, Cephalotaxaceae, Chenopodiaceae, Colchicaceae, Cucurbitaceae, Dioscoreaceae, Ephedraceae, Erythroxylaceae, Euphorbiaceae, Fabaceae, Lamiaceae, Linaceae, Lycopodiaceae, Malvaceae, Melanthiaceae, Musaceae, Myrtaceae, Nyssaceae, Papaveraceae, Pinaceae, Plantaginaceae, Poaceae, Rosaceae, Rubiaceae, Salicaceae, Sapindaceae, Solanaceae, Taxaceae, Theaceae, or Vitaceae.

Suitable species may include members of the genus Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

Suitable species include Panicum spp. or hybrids thereof, Sorghum spp. or hybrids thereof, sudangrass, Miscanthus spp. or hybrids thereof, Saccharum spp. or hybrids thereof, Erianthus spp., Populus spp., Andropogon gerardii (big bluestem), Pennisetum purpureum (elephant grass) or hybrids thereof (e.g., Pennisetum purpureum×Pennisetum typhoidum), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed) or hybrids thereof, Secale cereale (rye), Salix spp. (willow), Eucalyptus spp. (eucalyptus), Triticosecale (Triticum—wheat X rye), Tripsicum dactyloides (Eastern gammagrass), Leymus cinereus (basin wildrye), Leymus condensatus (giant wildrye), and bamboo.

In some embodiments, a suitable species can be a wild, weedy, or cultivated sorghum species such as, but not limited to, Sorghum almum, Sorghum amplum, Sorghum angustum, Sorghum arundinaceum, Sorghum bicolor (such as bicolor, guinea, caudatum, kafir, and durra), Sorghum brachypodum, Sorghum bulbosum, Sorghum burmahicum, Sorghum controversum, Sorghum drummondii, Sorghum ecarinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum propinquum, Sorghum purpureosericeum, Sorghum stipoideum, Sorghum sudanensese, Sorghum timorense, Sorghum trichocladum, Sorghum versicolor, Sorghum virgatum, Sorghum vulgare, or hybrids such as Sorghum×almum, Sorghum×sudangrass or Sorghum×drummondii.

Suitable species also include Helianthus annuus (sunflower), Carthamus tinctorius (safflower), Jatropha curcas (jatropha), Ricinus communis (castor), Elaeis guineensis (palm), Linum usitatissimum (flax), and Brassica juncea.

Suitable species also include Beta vulgaris (sugarbeet), and Manihot esculenta (cassava).

Suitable species also include Lycopersicon esculentum (tomato), Lactuca sativa (lettuce), Musa paradisiaca (banana), Solanum tuberosum (potato), Brassica oleracea (broccoli, cauliflower, brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), and Solanum melongena (eggplant).

Suitable species also include Papaver somniferum (opium poppy), Papaver orientale, Taxus baccata, Taxus brevifolia, Artemisia annua, Cannabis sativa, Camptotheca acuminate, Catharanthus roseus, Vinca rosea, Cinchona officinalis, Colchicum autumnale, Veratrum californica, Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (=Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, and Tanacetum parthenium.

Suitable species also include Parthenium argentatum (guayule), Hevea spp. (rubber), Mentha spicata (mint), Mentha piperita (mint), Bixa orellana, and Alstroemeria spp.

Suitable species also include Rosa spp. (rose), Dianthus caryophyllus (carnation), Petunia spp. (petunia) and Poinsettia pulcherrima (poinsettia).

Suitable species also include Nicotiana tabacum (tobacco), Lupinus albus (lupin), Uniola paniculata (oats), bentgrass (Agrostis spp.), Populus tremuloides (aspen), Pinus spp. (pine), Abies spp. (fir), Acer spp. (maple, Hordeum vulgare (barley), Poa pratensis (bluegrass), Lolium spp. (ryegrass) and Phleum pratense (timothy).

Thus, the methods and compositions can be used over a broad range of plant species, including species from the dicot genera Brassica, Carthamus, Glycine, Gossypium, Helianthus, Jatropha, Parthenium, Populus, and Ricinus; and the monocot genera Elaeis, Festuca, Hordeum, Lolium, Oryza, Panicum, Pennisetum, Phleum, Poa, Saccharum, Secale, Sorghum, Triticosecale, Triticum, and Zea. In some embodiments, a plant is a member of the species Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), or Pennisetum glaucum (pearl millet).

In certain embodiments, the polynucleotides and vectors described herein can be used to transform a number of monocotyledonous and dicotyledonous plants and plant cell systems, wherein such plants are hybrids of different species or varieties of a specific species (e.g., Saccharum sp. X Miscanthus sp., Saccharum sp. X Sorghum sp., Panicum virgatum×Panicum amarum, Panicum virgatum×Panicum amarulum, and Pennisetum purpureum×Pennisetum typhoidum).

D. Transgenic Plant Phenotypes

In some embodiments, a plant in which expression of a salinity and/or oxidative stress modulating polypeptide is modulated can have increased levels of tolerance to salinity and/or oxidative stress. For example, a salinity and/or oxidative stress-modulating polypeptide described herein can be expressed in a transgenic plant, resulting in increased levels of tolerance to salinity and/or oxidative stress. The salinity and/or oxidative stress tolerance levels can be increased by at least 0.25 percent, e.g., 0.25, 0.50, 0.75, 1, 2, 3, 4, 5, 6, 7, 8, 9, 10, 11, 12, 13, 14, 15, 16, 17, 18, 19, 20, 25, 30, 35, 40, 45, 50, 55, 60, 70, 80, 90, 100, 200 or more than 200 percent, as compared to those levels in a corresponding control plant that does not express the transgene. For example, plants as described herein show, under oxidative conditions, increased photosynthetic efficiency and increased seedling area as compared to a plant of the same species that is not genetically modified for substantial vegetative growth. Examples of increases in biomass production include increases of at least 5%, at least 20%, or even at least 50%, when compared to an amount of biomass production by a wild-type plant of the same species under identical conditions. For example, plants transformed with the sequences described herein can exhibit increases in SGI, seedling area and/or SAGI values of at least 5%, at least 10%, at least 25%, at least 50%, at least 75%, at least 100%, at least 200%, at least 300%, at least 400%, or even at least 500%. In some embodiments, plants described herein comprising the transgene corresponding to SEQ ID NO: 316 or 764 show, under normal growing conditions, increased biomass, increased photosynthetic efficiency, increased ry weight, and/or increased seedling area as compared to a plant of the same species that is not genetically modified for substantial vegetative growth.

In some embodiments, a plant in which expression of a salinity and/or oxidative stress tolerance-modulating polypeptide is modulated can be exposed to salinity and/or oxidative stress conditions for one or more periods of time that may vary depending on climatic conditions. For example, for periods of about ½ hour, 1 hour, 3 hours, 6 hours, 12 hours, 1 day, 3 days, 5 days, 10 days, 1 month, 3 months, 6 months, 12 months, or the entire lifespan of such a plant.

The nucleic acid molecules and polypeptides described herein are of interest because when the nucleic acid molecules are mis-expressed (i.e., when expressed at a non-natural location or in an increased or decreased amount relative to wild-type) they produce plants that exhibit improved salt tolerance and/or oxidation tolerance as compared to wild-type plants, as evidenced in part by the results of various experiments disclosed below. In particular, plants transformed with the nucleic acid molecules and polypeptides described herein can have any of a number of modified characteristics as compared to wild-type plants. Examples of modified characteristics include photosynthetic efficiency, seedling area, and biomass as it may be measured by plant height, leaf or rosette area, or dry mass. The modified characteristics may be observed and measured at different plant developmental stages, e.g. seed, seedling, bolting, senescense, etc. Often, salt or oxidative tolerance can be expressed as ratios or combinations of measurements, such as salt growth index values, or salicylic acid growth index values. For example, plants transformed with the sequences described herein can exhibit increases in SGI, seedling area and/or SAGI values of at least 5%, at least 10%, at least 25%, at least 50%, at least 75%, at least 100%, at least 200%, at least 300%, at least 400%, or even at least 500%. These traits can be used to exploit or maximize plant products. For example, the nucleic acid molecules and polypeptides described herein are used to increase the expression of genes that cause the plant to have improved biomass, growth rate and/or seedling vigor in saline and/or oxidative conditions, in comparison to wild type plants under the same conditions.

Because the disclosed sequences and methods increase vegetative growth and growth rate in saline and/or oxidative conditions, the disclosed methods can be used to enhance plant growth in plants grown in saline and/or oxidative conditions. For example, plants described herein show, under saline and/or oxidative conditions, increased photosynthetic efficiency and increased seedling area as compared to a plant of the same species that is not genetically modified for substantial vegetative growth. Examples of increases in biomass production include increases of at least 5%, at least 20%, or even at least 50%, when compared to an amount of biomass production by a wild-type plant of the same species under identical conditions.

Typically, a difference in the amount of tolerance to salinity and/or oxidative stress in a transgenic plant or cell relative to a control plant or cell is considered statistically significant at p≦0.05 with an appropriate parametric or non-parametric statistic, e.g., Chi-square test, Student's t-test, Mann-Whitney test, or F-test. In some embodiments, a difference in the amount of tolerance to salinity and/or oxidative stress is statistically significant at p<0.05, p<0.01, p<0.005, or p<0.001.

The phenotype of a transgenic plant is evaluated relative to a control plant. A plant is said “not to express” a polypeptide when the plant exhibits less than 10%, e.g., less than 9%, 8%, 7%, 6%, 5%, 4%, 3%, 2%, 1%, 0.5%, 0.1%, 0.01%, or 0.001%, of the amount of polypeptide or mRNA encoding the polypeptide exhibited by the plant of interest. Expression can be evaluated using methods including, for example, RT-PCR, Northern blots, S1 RNase protection, primer extensions, Western blots, protein gel electrophoresis, immunoprecipitation, enzyme-linked immunoassays, chip assays, and mass spectrometry. It should be noted that if a polypeptide is expressed under the control of a tissue-preferential or broadly expressing promoter, expression can be evaluated in the entire plant or in a selected tissue. Similarly, if a polypeptide is expressed at a particular time, e.g., at a particular time in development or upon induction, expression can be evaluated selectively at a desired time period.

### V. PLANT BREEDING

Genetic polymorphisms are discrete allelic sequence differences in a population. Typically, an allele that is present at 1% or greater is considered to be a genetic polymorphism. The discovery that polypeptides disclosed herein can modulate salinity and/or oxidative stress tolerance content is useful in plant breeding, because genetic polymorphisms exhibiting a degree of linkage with loci for such polypeptides are more likely to be correlated with variation in a salinity and/or oxidative stress tolerance trait. For example, genetic polymorphisms linked to the loci for such polypeptides are more likely to be useful in marker-assisted breeding programs to create lines having a desired modulation in the salinity and/or oxidative stress tolerance traits.

Thus, one aspect of the invention includes methods of identifying whether one or more genetic polymorphisms are associated with variation in a salinity and/or oxidative stress tolerance trait. Such methods involve determining whether genetic polymorphisms in a given population exhibit linkage with the locus for one of the polypeptides depicted in FIGS. 1 thru 6 and/or a functional homolog thereof, such as, but not limited to, those identified in the Sequence Listing. The correlation is measured between variation in the salinity and/or oxidative stress tolerance traits in plants of the population and the presence of the genetic polymorphism(s) in plants of the population, thereby identifying whether or not the genetic polymorphism(s) are associated with variation for the traits. If the presence of a particular allele is statistically significantly correlated with a desired modulation in the salinity and/or oxidative stress tolerance traits, the allele is associated with variation for one or both of the traits and is useful as a marker for one or more of the traits. If, on the other hand, the presence of a particular allele is not significantly correlated with the desired modulation, the allele is not associated with variation for one or more of the traits and is not useful as a marker.

Such methods are applicable to populations containing the naturally occurring endogenous polypeptide rather than an exogenous nucleic acid encoding the polypeptide, i.e., populations that are not transgenic for the exogenous nucleic acid. It will be appreciated, however, that populations suitable for use in the methods may contain a transgene for another, different trait, e.g., herbicide resistance.

Genetic polymorphisms that are useful in such methods include simple sequence repeats (SSRs, or microsatellites), rapid amplification of polymorphic DNA (RAPDs), single nucleotide polymorphisms (SNPs), amplified fragment length polymorphisms (AFLPs) and restriction fragment length polymorphisms (RFLPs). SSR polymorphisms can be identified, for example, by making sequence specific probes and amplifying template DNA from individuals in the population of interest by PCR. For example, PCR techniques can be used to enzymatically amplify a genetic marker associated with a nucleotide sequence conferring a specific trait (e.g., nucleotide sequences described herein). PCR can be used to amplify specific sequences from DNA as well as RNA, including sequences from total genomic DNA or total cellular RNA. When using RNA as a source of template, reverse transcriptase can be used to synthesize complementary DNA (cDNA) strands. Various PCR methods are described, for example, in PCR Primer: A Laboratory Manual, Dieffenbach and Dveksler, eds., Cold Spring Harbor Laboratory Press, 1995.

Generally, sequence information from polynucleotides flanking the region of interest or beyond is employed to design oligonucleotide primers that are identical or similar in sequence to opposite strands of the template to be amplified. Primers are typically 14 to 40 nucleotides in length, but can range from 10 nucleotides to hundreds of nucleotides in length. Template and amplified DNA is repeatedly denatured at a high temperature to separate the double strand, then cooled to allow annealing of primers and the extension of nucleotide sequences through the microsatellite, resulting in sufficient DNA for detection of PCR products. If the probes flank an SSR in the population, PCR products of different sizes will be produced. See, e.g., U.S. Pat. No. 5,766,847.

PCR products can be qualitative or quantitatively analyzed using several techniques. For example, PCR products can be stained with a fluorescent molecule (e.g., PicoGreen® or OliGreen®) and detected in solution using spectrophotometry or capillary electrophoresis. In some cases, PCR products can be separated in a gel matrix (e.g., agarose or polyacrylamide) by electrophoresis, and size-fractionated bands comprising PCR products can be visualized using nucleic acid stains. Suitable stains can fluoresce under UV light (e.g., Ethidium bromide, GR Safe, SYBR® Green, or SYBR® Gold). The results can be visualized via transillumination or epi-illumination, and an image of the fluorescent pattern can be acquired using a camera or scanner, for example. The image can be processed and analyzed using specialized software (e.g., ImageJ) to measure and compare the intensity of a band of interest against a standard loaded on the same gel.

Alternatively, SSR polymorphisms can be identified by using PCR product(s) as a probe against Southern blots from different individuals in the population. See, U. H. Refseth et al., (1997) Electrophoresis 18: 1519. Briefly, PCR products are separated by length through gel electrophoresis and transferred to a membrane. SSR-specific DNA probes, such as oligonucleotides labeled with radioactive, fluorescent, or chromogenic molecules, are applied to the membrane and hybridize to bound PCR products with a complementary nucleotide sequence. The pattern of hybridization can be visualized by autoradiography or by development of color on the membrane, for example.

In some cases, PCR products can be quantified using a real-time thermocycler detection system. For example, Quantitative real-time PCR can use a fluorescent dye that forms a DNA-dye-complex (e.g., SYBR® Green), or a fluorophore-containing DNA probe, such as single-stranded oligonucleotides covalently bound to a fluorescent reporter or fluorophore (e.g. 6-carboxyfluorescein or tetrachlorofluorescin) and quencher (e.g., tetramethylrhodamine or dihydrocyclopyrroloindole tripeptide minor groove binder). The fluorescent signal allows detection of the amplified product in real time, thereby indicating the presence of a sequence of interest, and allowing quantification of the copy number of a sequence of interest in cellular DNA or expression level of a sequence of interest from cellular mRNA.

The identification of RFLPs is discussed, for example, in Alonso-Blanco et al. (Methods in Molecular Biology, vol. 82, “Arabidopsis Protocols”, pp. 137-146, J. M. Martinez-Zapater and J. Salinas, eds., c. 1998 by Humana Press, Totowa, N.J.); Burr (“Mapping Genes with Recombinant Inbreds”, pp. 249-254, in Freeling, M. and V. Walbot (Ed.), The Maize Handbook, c. 1994 by Springer-Verlag New York, Inc.: New York, N.Y., USA; Berlin Germany; Burr et al. Genetics (1998) 118: 519; and Gardiner, J. et al., (1993) Genetics 134: 917). For example, to produce a RFLP library enriched with single- or low-copy expressed sequences, total DNA can be digested with a methylation-sensitive enzyme (e.g., PstI). The digested DNA can be separated by size on a preparative gel. Polynucleotide fragments (500 to 2000 bp) can be excised, eluted and cloned into a plasmid vector (e.g., pUC18). Southern blots of plasmid digests can be probed with total sheared DNA to select clones that hybridize to single- and low-copy sequences. Additional restriction endonucleases can be tested to increase the number of polymorphisms detected.

The identification of AFLPs is discussed, for example, in EP 0 534 858 and U.S. Pat. No. 5,878,215. In general, total cellular DNA is digested with one or more restriction enzymes. Restriction halfsite-specific adapters are ligated to all restriction fragments and the fragments are selectively amplified with two PCR primers that have corresponding adaptor and restriction site specific sequences. The PCR products can be visualized after size-fractionation, as described above.

In some embodiments, the methods are directed to breeding a plant line. Such methods use genetic polymorphisms identified as described above in a marker assisted breeding program to facilitate the development of lines that have a desired alteration in the salinity and/or oxidative stress tolerance trait(s). Once a suitable genetic polymorphism is identified as being associated with variation for the trait, one or more individual plants are identified that possess the polymorphic allele correlated with the desired variation. Those plants are then used in a breeding program to combine the polymorphic allele with a plurality of other alleles at other loci that are correlated with the desired variation. Techniques suitable for use in a plant breeding program are known in the art and include, without limitation, backcrossing, mass selection, pedigree breeding, bulk selection, crossing to another population and recurrent selection. These techniques can be used alone or in combination with one or more other techniques in a breeding program. Thus, each identified plants is selfed or crossed a different plant to produce seed which is then germinated to form progeny plants. At least one such progeny plant is then selfed or crossed with a different plant to form a subsequent progeny generation. The breeding program can repeat the steps of selfing or outcrossing for an additional 0 to 5 generations as appropriate in order to achieve the desired uniformity and stability in the resulting plant line, which retains the polymorphic allele. In most breeding programs, analysis for the particular polymorphic allele will be carried out in each generation, although analysis can be carried out in alternate generations if desired.

In some cases, selection for other useful traits is also carried out, e.g., selection for fungal resistance or bacterial resistance. Selection for such other traits can be carried out before, during or after identification of individual plants that possess the desired polymorphic allele.

### VI. ARTICLES OF MANUFACTURE

Transgenic plants provided herein have various uses in the agricultural and energy production industries. For example, transgenic plants described herein can be used to make animal feed and food products. Such plants, however, are often particularly useful as a feedstock for energy production.

Transgenic plants described herein often produce higher yields of grain and/or biomass per hectare, relative to control plants that lack the exogenous nucleic acid. In some embodiments, such transgenic plants provide equivalent or even increased yields of grain and/or biomass per hectare relative to control plants when grown under conditions of reduced inputs such as fertilizer and/or water. Thus, such transgenic plants can be used to provide yield stability at a lower input cost and/or under environmentally stressful conditions such as drought. In some embodiments, plants described herein have a composition that permits more efficient processing into free sugars, and subsequently ethanol, for energy production. In some embodiments, such plants provide higher yields of ethanol, butanol, dimethyl ether, other biofuel molecules, and/or sugar-derived co-products per kilogram of plant material, relative to control plants. By providing higher yields at an equivalent or even decreased cost of production relative to controls, the transgenic plants described herein improve profitability for farmers and processors as well as decrease costs to consumers.

Seeds from transgenic plants described herein can be conditioned and bagged in packaging material by means known in the art to form an article of manufacture. Packaging material such as paper and cloth are well known in the art. A package of seed can have a label, e.g., a tag or label secured to the packaging material, a label printed on the packaging material, or a label inserted within the package, that describes the nature of the seeds therein.

Enhanced salintiy and/or oxidative stress tolerance gives the opportunity to grow crops in saline or oxidative stress conditions without stunted growth and diminished yields due to salt-induced ion imbalance, disruption of water homeostasis, inhibition of metabolism, damage to membranes, and/or cell death. The ability to grow plants in saline or oxidative stress conditions would result in an overall expansion of arable land and increased output of land currently marginally productive due to elevated salinity or oxidative stress conditions.

Seed or seedling vigor is an important characteristic that can greatly influence successful growth of a plant, such as crop plants. Adverse environmental conditions, such as saline and/or oxidative conditions, can affect a plant growth cycle, germination of seeds and seedling vigor (i.e. vitality and strength under such conditions can differentiate between successful and failed plant growth). Seedling vigor has often been defined to comprise the seed properties that determine “the potential for rapid, uniform emergence and development of normal seedlings under a wide range of field conditions”. Hence, it would be advantageous to develop plant seeds with increased vigor, particularly in elevated salinity and/or in oxidative stress conditions.

For example, increased seedling vigor would be advantageous for cereal plants such as rice, maize, wheat, etc. production. For these crops, germination and growth can often be slowed or stopped by salination and/or oxidation. Genes associated with increased seed vigor under saline and/or oxidative stress conditions have therefore been sought for producing improved plant varieties. (Walia et al. (2005) Plant Physiology 139:822-835).

The invention will be further described in the following examples, which do not limit the scope of the invention described in the claims.

### VII. EXAMPLES

### Example 1

### Agrobacterium-Mediated Transformation of Arabidopsis

Host Plants and Transgenes: Wild-type Arabidopsis thaliana Wassilewskija (WS) plants were independently transformed with Ti plasmids containing clones encoding polypeptides at SEQ ID NOs: 2, 11, 102, 32, 138, 149, 441, 172, 764, 389, 316, 200, 697, 239, 892, 922, and 693. Examples include Ceres SEEDLINE ID no. ME24091, Ceres SEEDLINE ID no. ME11932, Ceres SEEDLINE ID no. ME02927, Ceres SEEDLINE ID no. ME08052, Ceres SEEDLINE ID no. ME02441, Ceres SEEDLINE ID no. ME03047, Ceres SEEDLINE ID no. ME03280, Ceres SEEDLINE ID no. ME08564, Ceres SEEDLINE ID no. ME01395, Ceres SEEDLINE ID no. ME06336, Ceres LOCUS ID no. At4g00150, Ceres LOCUS ID no. At3g45100, Ceres CLONE ID no. 547495, Ceres CLONE ID no. 560692, Ceres CLONE ID no. 22538, Ceres CLONE ID no. 210210, and Ceres SEEDLINE ID no. ME07302.

Unless otherwise indicated, each Ceres Clone and/or Seedline derived from a Clone is in the sense orientation relative to either the 35S promoter in a Ti plasmid. A Ti plasmid vector useful for these constructs, CRS 338, contains the Ceres-constructed, plant selectable marker gene phosphinothricin acetyltransferase (PAT), which confers herbicide resistance to transformed plants. The following is a list of nucleic acids that were isolated from Arabidopsis thaliana plants, Clone 151087 (ME06336) (SEQ ID NO: 439), AT3G20370 (ME03280) (SEQ ID NO: 387), At2g25920 (ME11932) (SEQ ID NO: 891), At4g00150 (ME17768) (SEQ ID NO: 148), At3g45100 (ME10409) (SEQ ID NO: 137), Clone 38456 (ME03047) (SEQ ID NO: 315), Clone 118036 (ME07302) (SEQ ID NO: 691), Clone 109156 (ME02441) (SEQ ID NO: 199), Clone 26652 (ME02927) (SEQ ID NO: 238), Clone 106263 (ME24091) (SEQ ID NO: 921), and Clone 22538 (ME03365) (SEQ ID NO: 10). The nucleic acids designated Clone 234510 (ME06336) (SEQ ID NO: 439), Clone 210210 (ME12933) (SEQ ID NO: 1) and Clone 311714 (ME08052) (SEQ ID NO: 695) were isolated from the species Zea mays. The nucleic acids designated Clone 554359 (ME08564) (SEQ ID NO: 762) and Clone 560692 (ME08322) (SEQ ID NO: 101) were isolated from the species Glycine max.

The following symbols are used in the Examples with respect to Arabidopsis transformation: T₁: first generation transformant; T₂: second generation, progeny of self-pollinated T₁ plants; T₃: third generation, progeny of self-pollinated T₂ plants; T₄: fourth generation, progeny of self-pollinated T₃ plants. Independent transformations are referred to as events.

Transgenic Arabidopsis lines containing Clones are designated by identifiers beginning with “ME” as indicated above. The presence of each vector containing a nucleic acid described above in the respective transgenic Arabidopsis line transformed with the vector is confirmed by Finale™ resistance, PCR amplification from green leaf tissue extract, and/or sequencing of PCR products. As controls, wild-type Arabidopsis ecotype Ws plants are transformed with the empty vector SR00559.

Preparation of Soil Mixture: 24 L Sunshine Mix #5 soil (Sun Gro Horticulture, Ltd., Bellevue, Wash.) is mixed with 16 L Therm-O-Rock vermiculite (Therm-O-Rock West, Inc., Chandler, Ariz.) in a cement mixer to make a 60:40 soil mixture. To the soil mixture is added 2 Tbsp Marathon 1% granules (Hummert, Earth City, Mo.), 3 Tbsp OSMOCOTE® 14-14-14 (Hummert, Earth City, Mo.) and 1 Tbsp Peters fertilizer 20-20-20 (J.R. Peters, Inc., Allentown, Pa.), which are first added to 3 gallons of water and then added to the soil and mixed thoroughly. Generally, 4-inch diameter pots are filled with soil mixture. Pots are then covered with 8-inch squares of nylon netting.

Planting: Using a 60 mL syringe, 35 mL of the seed mixture is aspirated. 25 drops are added to each pot. Clear propagation domes are placed on top of the pots that are then placed under 55% shade cloth and subirrigated by adding 1 inch of water.

Plant Maintenance: 3 to 4 days after planting, lids and shade cloth are removed. Plants are watered as needed. After 7-10 days, pots are thinned to 20 plants per pot using forceps. After 2 weeks, all plants are subirrigated with Peters fertilizer at a rate of 1 Tsp per gallon of water. When bolts are about 5-10 cm long, they are clipped between the first node and the base of stem to induce secondary bolts. Dipping infiltration is performed 6 to 7 days after clipping.

Preparation of Agrobacterium: To 150 mL fresh YEB is added 0.1 mL each of carbenicillin, spectinomycin and rifampicin (each at 100 mg/ml stock concentration). Agrobacterium starter blocks are obtained (96-well block with Agrobacterium cultures grown to an OD600 of approximately 1.0) and inoculated one culture vessel per construct by transferring 1 mL from appropriate well in the starter block. Cultures are then incubated with shaking at 27° C. Cultures are spun down after attaining an OD600 of approximately 1.0 (about 24 hours). 200 mL infiltration media is added to resuspend Agrobacterium pellets. Infiltration media is prepared by adding 2.2 g MS salts, 50g sucrose, and 5 μL 2 mg/ml benzylaminopurine to 900 ml water.

Dipping Infiltration: The pots are inverted and submerged for 5 minutes so that the aerial portion of the plant is in the Agrobacterium suspension. Plants are allowed to grow normally and seed is collected.

### Example 2

### Saline Condition Screening

Saline condition screening: Screening is routinely performed by high-salt agar plate assay and also by high-salt soil assay. Traits assessed in high-salt conditions include: seedling area, photosynthesis efficiency, salt growth index and regeneration ability.

Seedling area: the total leaf area of a young plant about 2 weeks old.

Photosynthesis efficiency (Fv/Fm): Seedling photosynthetic efficiency, or electron transport via photosystem II, is estimated by the relationship between Fm, the maximum fluorescence signal and the variable fluorescence, Fv. Here, a reduction in the optimum quantum yield (Fv/Fm) indicates stress, and so can be used to monitor the performance of transgenic plants compared to non-transgenic plants under salt stress conditions.

Salt growth index=seedling area×photosynthesis efficiency (Fv/Fm).

Regeneration ability: the ability of a plant to regenerate shoots in saline soil after stems are cut off and the soil is irrigated with 200 mM NaCl solution.

Transformant identification: PCR is used to amplify the cDNA insert in one randomly chosen T2 plant. This PCR product was then sequenced to confirm the sequence in the plants.

Identification of Tolerant Plant to Salt Stress: A superpool of seeds is screened for transgenic plants that show enhanced tolerance to SA, as detailed below, and high salt.

Assessing Tolerance to Salt Stress: Generally, between four and ten independently transformed plant lines are selected and qualitatively evaluated for their tolerance to salt stress in the T₁ generation. Two or three of the transformed lines that qualitatively show the strongest tolerance to salt stress in the T₁ generation are selected for further evaluation in the T₂ and T₃ generations. This evaluation involves sowing seeds from the selected transformed plant lines on MS agar plates containing either 100 mM or 150 mM NaCl and incubating the seeds for 5 to 14 days to allow for germination and growth. For example, five T₂ events may be compared to wild-type Ws for salt stress tolerance on salt plates. Three events, 01, -03 and -04 may be selected based on the measurement of seedling area on 36 plants of each event as compared to the control, Ws. Further evaluation of salt tolerance in -01, -03 and -04 might be performed with T₂ and T₃ generations.

Calculating SGI: After germination and growth, seedling area and photosynthesis efficiency of transformed lines and a wild-type control are determined. From these measurements, the Salt Growth Index (SGI) is calculated and compared between wild-type and transformed seedlings. The SGI calculation is made by multiplying seedling area with photosynthesis efficiency measurements taken from two replicates of 36 seedlings for each transformed line and a wild-type control and performing a t-test.

Determining Transgene Copy Number: T₂ generation transformed plants are tested on BASTATM plates in order to determine the transgene copy number of each transformed line. A BASTATM resistant:BASTATM sensitive segregation ratio of 15:1 generally indicates two copies of the transgene, and such a segregation ratio of 3:1 generally indicates one copy of the transgene.

### Example 3

### Oxidative Stress Conditions Screening

Under normal growth conditions, Arabidopsis rosette contains about 0.5 μg/g fresh weight of free SA. In response to stress conditions or pathogen attacks, the free SA levels can reach as high as 10 μg/g fresh weight, which is approximately equivalent to 60 μM. The exogenous application of 100-500 μM SA to Arabidopsis leaves by spraying is able to induce strong defense responses without triggering obvious necrotic lesion formation. Once the SA concentration increases to 5 mM or above, the cell death in form of necrotic lesions will appear on the sprayed leaves. If SA is applied through growth media, Arabidopsis is more sensitive to SA-induced oxidative stress, probably because of continuous absorption. The addition of 100-150 μM SA to growth media significant reduces plant growth but does not kills the plants in wild type Arabidopsis Ws. Therefore we use this range of SA to screen for enhanced oxidative stress tolerance.

### Salicylic Acid Screening:

Screening is routinely performed by agar plate assay using 100 μM or 150 μM exogenous sodium salicylate. Media contains 1/2X MS (Sigma), 150 μM sodium salicylate (Sigma), 0.5 g MES hydrate (Sigma) and 0.7% phytagar (EM Science), adjusted to pH 5.7 using 10N KOH.

To screen superpools, seeds are surface sterilized in 30% bleach solution for 5 minutes and then rinsed repeatedly with sterile water. Approximately 2500 seeds are sown on media plates in a monolayer at a density of 850 seeds per plate. Wild-type and positive controls are grown on comparable plates. Plates are wrapped with vent tape and placed at 4° C. in the dark for three days to stratify. At the end of this time, plates are transferred to a Conviron growth chamber set at 22° C., 16:8 hour light:dark cycle, 70% humidity with a combination of incandescent and fluorescent lamps emitting a light intensity of ˜100 μEinsteins.

Seedlings are screened daily starting at 6 days. Seedlings that grow larger and stay greener compared to WS control plants are selected as positive candidates and transferred to soil for recovery and seed set.

Candidate plants are re-screened by placing 36 seeds from each candidate together with a WS control on the same sodium salicylate plate. Plates are treated as described above and seedling screening begun after at 4 days after germination. Leaf tissue is harvested from confirmed tolerant candidates for DNA extraction and amplification of the transgene by PCR.

Alternatively, superpool seeds are sown directly on soil and sprayed with 10 mM SA. Leaf tissue is harvested from tolerant candidate plants to isolate DNA for PCR amplification of the transgene and subsequent sequencing of the PCR product.

Traits assessed under sodium salicylate conditions include: seedling area, photosynthesis efficiency, salicylic acid growth index (SAG) and regeneration ability.

Seedling area: the total leaf area of a young plant about 2 weeks old.

Photosynthesis efficiency (Fv/Fm): Seedling photosynthetic efficiency, or electron transport via photosystem II, is estimated by the relationship between Fm, the maximum fluorescence signal and the variable fluorescence, Fv. Here, a reduction in the optimum quantum yield (Fv/Fm) indicates stress, and so can be used to monitor the performance of transgenic plants compared to non-transgenic plants under oxidative stress conditions.

Salicylic Acid Growth (SAG) Index=seedling area (cm²)×photosynthesis efficiency (Fv/Fm).

PCR is used to amplify the cDNA insert in one randomly chosen T₂ plant. This PCR product is then sequenced to confirm the sequence in the plants.

Assessing Tolerance to Oxidative Stress: Initially, All available independently transformed T₂ plant lines are qualitatively evaluated for their tolerance to oxidative stress as compared to wild-type controls. The positive transgenic lines that qualitatively show the strongest tolerance to oxidative stress are selected for further evaluation in the T₂ and T₃ generations using internal non-transgenic segregants as controls. This evaluation involves sowing seeds from the selected transformed plant lines on MS agar plates containing 100 μM or 150 μM sodium salicylate and incubating the seeds for at least 4 days to allow for germination and growth and transgene status analysis.

Calculating SAG: After germination and growth, seedling area and photosynthesis efficiency of transformed lines and a wild-type control are determined. From these measurements, the Salicylic Acid Growth Index (SAG) is calculated and compared between wild-type and transformed seedlings. The SAG calculation is made by multiplying seedling area with photosynthesis efficiency measurements taken from two replicates of 36 seedlings for each transformed line and a wild-type control and performing a t-test.

Determining Transgene Copy Number: T₂ generation transformed plants are tested on BASTATM plates in order to determine the transgene copy number of each transformed line. A BASTATM resistant:BASTATM sensitive segregation ratio of 15:1 generally indicates two copies of the transgene, and such a segregation ratio of 3:1 generally indicates one copy of the transgene.

In some cases, validation is perfomed using media that is further supplemented with 100 uM SNP.

### L-arginine Screening:

Screening superpools for L-arginine (NO) tolerance: about twenty-five hundred seeds of Superpools were sown on MS agar plates containing 10 mM L-arginine (pH 9.0). One-week-old seedlings are screened for better growth on L-arginine media. The tolerant candidates are pulled out and transferred to soil and the seeds are collected from each candidate. The T3 progeny of tolerant candidates are tested on L-arginine media together with Ws controls. Leaf tissue is harvested from the most tolerant candidates to isolate DNA for PCR amplification of the transgene. Sequencing the PCR product is used to reveal which candidates are derived from ME lines.

Prevalidation and validation assays: For prevalidation, individual T₂ events of an ME line and wild-type Ws control are grown on the same plate containing 10 mM L-arginine (pH 5.7) in a small square format to compare their tolerance to L-arginine-induced oxidative stress. Thirteen seeds are sown in each square and the plate placed under normal growth conditions. Images of seedlings are taken at one week old. The positive events are selected based on the visual evaluation of growth performance in comparison to wild-type control.

In the validation assay, plants of the positive T₂ or T₃ events of an ME line and wild-type Ws plants are grown on MS agar plates containing 10 mM L-arginine (pH 5.7) for 7-14 days in a pre-determined configuration. The plants are scanned using an EPSON color scanner and chlorophyll fluorescence imager. The seedling area is measured for each plant. After scanning, the plants are transferred to fresh MS plates without L-arginine in the same configuration for recovery. One week after the transfer, the recovered plants are sprayed with Finale™ and transgenic status of each plant scored based on the sensitivity to Finale™. Two plates are used as independent replicates for each event per generation.

Sequence confirmation: The transgene in an ME line is amplified and sequenced from both sense and antisense orientations.

### Example 4

### ME01359 (Ceres Clone 151087; SEQ ID No.172)

ME01395 (from Arabidopsis thaliana) showed enhanced tolerance to oxidative stress induced by L-arginine. Wild-type Ws seedlings showed stunted growth on plates containing 10 mM L-arginine, whereas the transgenic plants showed significantly better growth.

ME01395 was identified from a superpool screen for L-arginine tolerance. Superpool 1 was screened for plants that showed enhanced tolerance to L-arginine using the assay described in Example 2. The progeny of the candidates were tested for L-arginine tolerance in a small square format. The selected tolerant candidates were sequenced and one of them matched the transgene sequence in ME01395. Prevalidation revealed four positive events of ME01395 in L-arginine tolerance. Five T₂ events of ME01395 were compared to wild-type Ws for L-arginine tolerance in a small square format. Four events (ME01395-01, -02, -04 and -05) showed visually better growth under L-arginine stress as compared to Ws. ME01395-02, -04 and -05 were selected for further validation assay in T₂ and T₃ generations.

ME01395 showed significantly better tolerance to L-arginine than wild-type Ws. ME01395-02, -04 and -05 T₂ plants were assayed in a validation configuration. The transgenic plants were compared to the internal non-transgenic plants as well as external controls that were grown on the same L-arginine plate. For each event, two independent replicates (36 seedlings each) were analyzed. Seedling area was measured for each plant. As shown in Table 1, the differences between transgenic and pooled non-transgenic plants are significant. These results indicate that the enhanced L-arginine stress tolerance is mediated by the transgene.

Functional analysis of the transgene in L-arginine-induced stress tolerance confirmed that the transgene caused the enhanced L-arginine tolerance, as T₃ lines of ME01395-02, -04 and -05 were further validated. As shown in Table 1, transgenic plants of all tested lines showed better growth than pooled non-transgenic plants under L-arginine stress. These results confirm that the observed L-arginine stress tolerance in ME01395 is mediated by the transgene.

TABLE 1
Validation assay of ME01395 on Arginine
tolerance in two generations.

| | | | | Seedling area of | | | | |
|---|---|---|---|---|---|---|---|---|
| | Seedling area | | | pooled non- | | | | |
| | of transgenics | | | transgenics | | | t-test | Increase |
| ME Events | Avg | SE | N | Avg | SE | N | (p-value) | (%) |
| ME01395- | 1.984 | 0.126 | 30 | 0.939 | 0.119 | 37 | 4.1E−08 | 211.4% |
| 02T₂ | | | | | | | | |
| ME01395- | 2.181 | 0.123 | 26 | 0.860 | 0.101 | 46 | 2.7E−12 | 253.4% |
| 04T₂ | | | | | | | | |
| ME01395- | 2.155 | 0.127 | 26 | 0.777 | 0.082 | 46 | 7.5E−14 | 277.3% |
| 05T₂ | | | | | | | | |
| ME01395- | 0.685 | 0.060 | 32 | 0.297 | 0.065 | 33 | 2.2E−05 | 230.8% |
| 02T₃ | | | | | | | | |
| ME01395- | 0.798 | 0.046 | 35 | 0.560 | 0.068 | 37 | 2.5E−03 | 142.5% |
| 04T₃ | | | | | | | | |
| ME01395- | 0.792 | 0.056 | 51 | 0.297 | 0.064 | 20 | 8.5E−08 | 267.2% |
| 05T₃ | | | | | | | | |

Events ME01395-02, -04 and -05 segregated for Finale™ resistance in the T₂ generation. ME01395 segregated 1:1 (R:S) for Finale™, whereas ME01395-04 and -05 segregated 3:1 (R:S), suggesting that there is likely one copy of the transgene in each event. Events -02, -04 and -05 of ME01395 are either hemizygous or homozygous for the transgene and exhibited no statistically relevant negative phenotypes in the traits being investigated compared to empty vector control SR00559 under standard growth conditions.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 5

### ME06336 (Ceres Clone 234510; SEQ ID No.441)

ME06336 (from Zea mays) showed enhanced tolerance to oxidative stress induced by salicylic acid (SA). Wild-type Ws seedlings showed necrotic lesions and stunted growth on plates containing 100 or 150 mM SA, whereas the transgenic plants showed significantly better growth.

ME06336 was identified from superpool screen for SA tolerance. Superpool 51 was screened for transgenic plants that show enhanced tolerance to SA (see Example 2). One candidate was sequenced and the transgene sequence matched ME06336. Two events of ME06336 showed significantly better tolerance to SA than wild-type Ws. In a prevalidation assay, five events of ME06336 (T₂) together with wild-type controls were tested on SA plates for oxidative stress tolerance. Two events, -12 and -13, showed significantly better growth on SA plates as compared to the control. The selected two positive events were further validated in T₂ and T₃ generations.

T₂ and T₃ validation assays confirmed the SA stress tolerance in ME06336. To confirm that the transgene causes enhanced SA tolerance, T₂ and T₃ generations of the two positive events, ME06336-12 and -13, were further validated. The transgenic plants were compared to the internal non-transgenic plants as well as external controls that were grown on the same SA plate. For each event by generation, two independent replicates (36 seedlings each) were analyzed. For each plant, seedling area was measured to reflect the growth rate under SA-induced oxidative stress (100 μM SA). As shown in Table 2, the T₂ transgenic plants of ME06336-12 and -13 grew significantly larger than pooled non-transgenic plants. In T₃ generation, three individual lines were tested for each event, and transgenic plants in all tested lines except ME06336-13-02 showed better tolerance to SA stress as compared to pooled non-transgenic controls. These results indicate that the enhanced SA stress tolerance is mediated by the transgene.

TABLE 2
Validation assay of ME06336 on SA tolerance in two generations

| | | | | Seedling area | | | | |
|---|---|---|---|---|---|---|---|---|
| | Seedling area | | | of pooled | | | | |
| | of transgenics | | | non-transgenics | | | t-test | Increase |
| ME Events | Avg | SE | N | Avg | SE | N | (p-value) | (%) |
| ME06336- | 0.556 | 0.035 | 37 | 0.355 | 0.041 | 35 | 0.0002 | 156.4% |
| 12T₂ | | | | | | | | |
| ME06336- | 0.498 | 0.038 | 42 | 0.331 | 0.035 | 30 | 0.0010 | 150.6% |
| 13T₂ | | | | | | | | |
| ME06336- | 1.089 | 0.059 | 58 | 0.504 | 0.151 | 12 | 0.0003 | 216.0% |
| 12T₃-01 | | | | | | | | |
| ME06336- | 0.439 | 0.047 | 53 | 0.203 | 0.054 | 18 | 0.0008 | 216.1% |
| 12T₃-05 | | | | | | | | |
| ME06336- | 0.579 | 0.051 | 42 | 0.365 | 0.053 | 28 | 0.0025 | 158.5% |
| 12T₃-06 | | | | | | | | |
| ME06336- | 0.397 | 0.057 | 38 | 0.387 | 0.058 | 27 | 0.4509 | 102.6% |
| 13T₃-02 | | | | | | | | |
| ME06336- | 0.627 | 0.062 | 44 | 0.442 | 0.078 | 28 | 0.0334 | 141.9% |
| 13T₃-03 | | | | | | | | |
| ME06336- | 0.817 | 0.064 | 39 | 0.483 | 0.076 | 30 | 0.0006 | 169.1% |
| 13T₃-04 | | | | | | | | |

Events ME06336-12 and -13 segregated for Finale™ resistance in the T₃ generation. Both segregate 3:1 (R:S) for Finale™, suggesting that there is only one copy of the transgene in each event. Events -12 and -13 of ME06336 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, or rosette area 7 days post-bolting.

### Example 6

### ME08564 (Ceres Clone 554359; SEQ ID No. 764)

ME08564 (from Glycine max) showed enhanced tolerance to oxidative stress induced by salicylic acid (SA) in Events -01 and -02. Wild-type Ws seedlings showed necrotic lesions and stunted growth on plates containing 100 to 150 μM SA, whereas the transgenic plants showed significantly better growth.

ME08564 was identified from superpool screen for SA tolerance. Superpool 66 was screened for transgenic plants that show enhanced tolerance to SA. The progeny of candidate SP66-2 was further tested for SA tolerance and confirmed to be positive. SP66-2 was sequenced and its transgene matched ME08564. Two events of ME08564 showed significantly better tolerance to SA than wild-type Ws. In a prevalidation assay, four events of ME08564 (T₂) together with wild-type controls were tested on SA plates for oxidative stress tolerance. Two events, -01 and -02, showed significantly better growth on SA plates as compared to the control. The selected two positive events were further validated in T₂ and T₃ generations.

T₂ and T₃ validation assays confirmed the SA stress tolerance in ME08564. To confirm that the transgene caused enhanced SA tolerance, T₂ and T₃ generations of the two positive events, ME08564-01 and -02, were further validated. The transgenic plants were compared to the internal non-transgenic plants, as well as external controls that were grown on the same SA plate. For each event by generation, two independent replicates (36 seedlings each) were analyzed. For each plant, seedling area was measured to reflect the growth rate under SA-induced oxidative stress (125 μM SA). As shown in Table 3, the T₂ transgenic plants of ME08564-01 and -02 grew significantly larger than pooled non-transgenic plants and the increases were 379.6 and 519.9%, respectively. The differences were repeated in T₃ generation. As compared to the pooled non-transgenic controls, the seedling areas of T₃ ME08564-01 and -02 plants increased 157.2 and 157.4%, respectively. These results indicated that the enhanced SA stress tolerance is mediated by the transgene.

TABLE 3
Validation assay of ME08564 on SA tolerance in two generations.

| | Seedling area | | | Seedling area | | | | |
|---|---|---|---|---|---|---|---|---|
| | of | | | of pooled | | | | |
| | transgenics | | | non-transgenics | | | t-test | Increase |
| ME Events | Avg | SE | N | Avg | SE | N | (p-value) | (%) |
| ME08564- | 0.470 | 0.046 | 42 | 0.124 | 0.034 | 24 | 3.9E−08 | 379.6% |
| 01T₂ | | | | | | | | |
| ME08564- | 0.595 | 0.045 | 47 | 0.115 | 0.030 | 20 | 9.3E−11 | 519.9% |
| 02T₂ | | | | | | | | |
| ME08564- | 0.627 | 0.062 | 43 | 0.399 | 0.069 | 22 | 0.008 | 157.2% |
| 01T₃ | | | | | | | | |
| ME08564- | 0.919 | 0.066 | 50 | 0.584 | 0.135 | 15 | 0.015 | 157.4% |
| 02T₃ | | | | | | | | |

Events ME08564-01 and -02 segregated for Finale™ resistance in the T₂ generation. Both segregate 3:1 (R:S) for Finale™, suggesting that there is only one copy of the transgene in each event. Events -01 and -02 of ME08564 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 7

### ME03280 (Ceres clone AT3G20370; SEQ ID No. 389)

ME03280 showed enhanced tolerance to oxidative stress induced by L-arginine Wild-type Ws seedlings showed stunted growth on plates containing 10 mM L-arginine, whereas the transgenic plants showed significantly better growth.

ME03280 was identified from a superpool screen for L-Arginine tolerance. Superpool 21 was screened for plants that showed enhanced tolerance to L-arginine. The progeny of the candidates were tested for L-Arginine tolerance in a small square format. The selected tolerant candidates were sequenced and one of them matched the transgene sequence in ME03280.

Prevalidation revealed four positive events of ME03280 mL-Arginine tolerance. Five T₂ events of ME03280 were compared to wild-type Ws for L-Arginine tolerance in a small square format. Four events (ME03280-01, -03, -04 and -05) showed visually better growth under L-Arginine stress as compared to Ws. The positive events were selected for further validation assay in T₂ and T₃ generations.

ME03280 showed significantly better tolerance to L-Arginine than wild-type Ws. ME03280-01, -03, -04 and -05 T₂ plants were assayed in a validation configuration. The transgenic plants were compared to the internal non-transgenic plants as well as external controls that were grown on the same L-Arginine plate. For each event, two independent replicates (36 seedlings each) were analyzed. Seedling area was measured for each plant. As shown in Table 1, the differences between transgenic and pooled non-transgenic plants are significant. These results indicate that the enhanced-L-arginine stress tolerance is mediated by the transgene.

To confirm that the transgene causes the enhanced-L-arginine tolerance, T₃ lines of ME03280-01, -04 and -05 were further validated. As shown in Table 4, transgenic plants of all tested lines showed better growth than pooled non-transgenic plants under L-Arginine-induced stress. These results confirm that the observed better L-arginine stress tolerance in ME03280 is mediated by the transgene.

TABLE 4
Validation assay of ME03280 on Arginine tolerance in two generations

| | Seedling area | | | Seedling | | | | |
|---|---|---|---|---|---|---|---|---|
| | of | | | area of pooled | | | | |
| | transgenics | | | non-transgenics | | | t-test | Increase |
| ME Events | Avg | SE | N | Avg | SE | N | (p-value) | (%) |
| ME03280- | 3.087 | 0.189 | 33 | 2.404 | 0.298 | 30 | 2.75E−02 | 128.4% |
| 01T₂ | | | | | | | | |
| ME03280- | 3.706 | 0.225 | 40 | 1.674 | 0.278 | 29 | 5.73E−08 | 221.4% |
| 03T₂ | | | | | | | | |
| ME03280- | 2.779 | 0.244 | 26 | 1.476 | 0.226 | 25 | 3.67E04 | 188.2% |
| 04T₂ | | | | | | | | |
| ME03280- | 2.845 | 0.167 | 32 | 2.148 | 0.225 | 35 | 1.06E−02 | 132.5% |
| 05T₂ | | | | | | | | |
| ME03280- | 3.900 | 0.245 | 30 | 2.120 | 0.283 | 41 | 2.05E−06 | 184.0% |
| 01T₃-02 | | | | | | | | |
| ME03280- | 3.218 | 0.218 | 36 | 1.763 | 0.264 | 34 | 4.28E−05 | 182.5% |
| 01T₃-03 | | | | | | | | |
| ME03280- | 3.472 | 0.191 | 35 | 1.616 | 0.312 | 36 | 1.48E−07 | 214.8% |
| 04T₃-01 | | | | | | | | |
| ME03280- | 3.282 | 0.246 | 32 | 1.234 | 0.215 | 26 | 6.41E−07 | 266.0% |
| 04T₃-02 | | | | | | | | |
| ME03280- | 3.568 | 0.278 | 26 | 2.050 | 0.281 | 46 | 3.80E−05 | 174.1% |
| 05T₃-01 | | | | | | | | |
| ME03280- | 3.530 | 0.208 | 41 | 3.023 | 0.262 | 31 | 6.72E−02 | 116.8% |
| 05T₃-02 | | | | | | | | |

Events ME03280-01 and -05 s segregated for Finale™ resistance in the T₂ generation. Both segregate 3:1 (R:S) for Finale™, suggesting that there is only one copy of the transgene in each event. Events -01 and -05 of ME03280 exhibited no statistically relevant negative phenotypes for the tested traits.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 8

### ME03047 (Ceres Clone 38456; SEQ ID No. 316)

ME03047 showed enhanced tolerance to oxidative stress induced by L-arginine. Wild-type Ws seedlings showed stunted growth on plates containing 10 mM L-arginine, whereas the transgenic plants grew significantly better.

ME03047 was a homolog of a candidate gene identified from a superpool screen for L-arginine tolerance. Superpool 20 was screened for plants that showed enhanced tolerance to L-arginine. The progeny of the candidates were tested for L-arginine tolerance in a small square format. The selected tolerant candidates were sequenced and one of them matched the transgene sequence in ME02927.

Prevalidation revealed four positive events of ME03047 mL-arginine tolerance. Five T₂ events of ME03047 were compared to wild-type Ws for L-arginine tolerance in a small square format. Four events (ME03047-01, -03 and -05) showed visually better growth under L-arginine stress as compared to Ws. The positive events were selected for further validation assay in T₂ and T₃ generations.

ME03047 showed significantly better tolerance to L-arginine than wild-type Ws. ME03047-01, -03 and -05 T₂ plants were assayed in a validation configuration. The transgenic plants were compared to the internal non-transgenic plants as well as external controls that were grown on the same L-arginine plate. For each event, two independent replicates (36 seedlings each) were analyzed. Seedling area was measured for each plant. As shown in Table 1, the differences between transgenic and pooled non-transgenic plants are significant. These results indicated that the enhanced-L-arginine stress tolerance is mediated by the transgene.

The functional confirmation of the transgene in L-arginine-induced stress tolerance. To confirm that the transgene causes the enhanced-L-arginine tolerance, two individual T₃ lines of each positive event were further validated. As shown in Table 5, transgenic plants of all tested lines showed better growth than pooled non-transgenic plants under L-arginine-induced stress. These results confirm that the observed better L-arginine stress tolerance in ME03047 is mediated by the transgene.

TABLE 5
Validation assay of ME03047 on arginine tolerance in two generations.

| | Seedling area | | | Seedling area of | | | | |
|---|---|---|---|---|---|---|---|---|
| | of | | | pooled non- | | | | |
| | transgenics | | | transgenics | | | t-test | Increase |
| ME Events | Avg | SE | N | Avg | SE | N | (p-value) | (%) |
| ME03047- | 3.453 | 0.136 | 36 | 2.218 | 0.244 | 34 | 1.87E−05 | 155.7% |
| 01T₂ | | | | | | | | |
| ME03047- | 3.144 | 0.184 | 32 | 1.518 | 0.228 | 32 | 3.18E−07 | 207.1% |
| 03T₂ | | | | | | | | |
| ME03047- | 4.193 | 0.166 | 38 | 2.520 | 0.341 | 28 | 1.99E−05 | 166.4% |
| 05T₂ | | | | | | | | |
| ME03047- | 4.778 | 0.153 | 37 | 2.935 | 0.357 | 35 | 5.36E−06 | 162.8% |
| 01T₃-01 | | | | | | | | |
| ME03047- | 5.048 | 0.281 | 35 | 1.775 | 0.315 | 37 | 2.60E−11 | 284.5% |
| 01T₃-02 | | | | | | | | |
| ME03047- | 4.278 | 0.150 | 39 | 3.096 | 0.313 | 33 | 5.50E−04 | 138.2% |
| 03T₃-01 | | | | | | | | |
| ME03047- | 4.044 | 0.227 | 38 | 2.264 | 0.328 | 34 | 1.49E−05 | 178.6% |
| 03T₃-02 | | | | | | | | |
| ME03047- | 4.576 | 0.181 | 35 | 2.995 | 0.320 | 37 | 2.78E−05 | 152.8% |
| 05T₃-01 | | | | | | | | |
| ME03047- | 5.184 | 0.224 | 39 | 4.214 | 0.362 | 33 | 1.28E−02 | 123.0% |
| 05T₃-02 | | | | | | | | |

Events ME03047-01 and -05 segregated for Finale™ resistance in the T₂ generation. Both segregate 3:1 (R:S) for Finale™, suggesting that there is only one copy of the transgene in each event. Events -01 and -05 of ME03047 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 9

### ME02441 (Ceres Clone 109156; SEQ ID No. 200)

ME02441 showed enhanced tolerance to oxidative stress induced by L-arginine ME02441 was identified from a superpool screen for L-arginine tolerance. Superpool 06 was screened for plants that showed enhanced tolerance to L-arginine. The progeny of the candidates were tested for L-arginine tolerance in a small square format. The selected tolerant candidates were sequenced and one of them matched the transgene sequence in ME02441.

Prevalidation revealed two positive events of ME02441 mL-arginine tolerance. Five T₂ events of ME02441 were compared to wild-type Ws for L-arginine tolerance in a small square format. Three events (ME02441-02, -04 and -05) showed visually better growth under L-arginine stress as compared to Ws. The positive events were selected for further validation assay in T₂ and T3₂ generations.

ME02441 showed significantly better tolerance to L-arginine than wild-type Ws. ME02441-02, -04 and -05 T₂ plants were assayed in a validation configuration. The transgenic plants were compared to the internal non-transgenic plants as well as external controls that were grown on the same L-arginine plate. For each event, two independent replicates (36 seedlings each) were analyzed. Seedling area was measured for each plant. As shown in Table 6, the differences between transgenic and pooled non-transgenic plants were significant. These results indicate that the enhanced-L-arginine stress tolerance is mediated by the transgene.

The functional confirmation of the transgene in L-arginine-induced stress tolerance. To confirm that the transgene caused the enhanced L-arginine tolerance, three independent T₃ lines of ME02441-02, -04 and -05 were further validated. As shown in Table 6, transgenic plants of ME02441-02-1, -02-02, -02-03, -05-01 and -05-03 showed better growth than pooled non-transgenic plants under L-arginine-induced stress. These results confirmed that the observed better L-arginine stress tolerance in ME02441 is mediated by the transgene.

TABLE 6
Validation assay of ME02441 on arginine tolerance in two generations.

| | | | | Seedling area of | | | | |
|---|---|---|---|---|---|---|---|---|
| | Seedling area of | | | pooled non- | | | | |
| | transgenics | | | transgenics | | | t-test | Increase |
| ME Events | Avg | SE | N | Avg | SE | N | (p-value) | (%) |
| ME02441-02T₂ | 1.30 | 0.072 | 38 | 0.95 | 0.082 | 34 | 0.0010 | 36.89% |
| ME02441-04T₂ | 1.38 | 0.078 | 37 | 1.00 | 0.102 | 34 | 0.0021 | 38.05% |
| ME02441-05T₂ | 1.23 | 0.077 | 43 | 0.91 | 0.093 | 28 | 0.0050 | 35.31% |
| ME02441-02T₃-01 | 5.89 | 0.328 | 33 | 4.05 | 0.400 | 30 | 0.0004 | 45.54% |
| ME02441-02T₃-02 | 5.03 | 0.273 | 33 | 4.11 | 0.320 | 32 | 0.0158 | 22.48% |
| ME02441-02T₃-03 | 5.23 | 0.296 | 39 | 3.63 | 0.354 | 31 | 0.0004 | 44.13% |
| ME02441-04T₃-01 | 4.51 | 0.328 | 36 | 5.21 | 0.515 | 27 | 0.1281 | −13.43% |
| ME02441-04T₃-02 | 4.86 | 0.418 | 25 | 4.78 | 0.272 | 50 | 0.4345 | 1.73% |
| ME02441-04T₃-03 | 4.16 | 0.395 | 19 | 4.71 | 0.336 | 30 | 0.1454 | −11.75% |
| ME02441-05T₃-01 | 6.57 | 0.443 | 23 | 4.45 | 0.590 | 11 | 0.0035 | 47.79% |
| ME02441-05T₃-02 | 5.65 | 0.498 | 39 | 4.76 | 0.546 | 28 | 0.1168 | 18.68% |
| ME02441-05T₃-03 | 4.30 | 0.324 | 28 | 2.67 | 0.521 | 7 | 0.0061 | 60.95% |

Events ME02441-02 and -05 segregated for Finale™ resistance in the T₂ generation. ME02441-02 segregates 2:1 (R:S) for Finale™ and ME02441-05 segregates 3:1 (R:S), suggesting that there is likely one copy of the transgene in each event, but the transgene inheritance was effected by integration site. Events ME02441-02 and -05 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 10

### ME08052 (Ceres Clone 311714; SEQ ID No. 697)

ME08052 showed enhanced tolerance to oxidative stress induced by salicylic acid (SA) in Events -02 and -05 of both T₂ and T₃ generation. ME08052 was identified from superpool screen for SA tolerance. Superpool 62 was screened for transgenic plants that show enhanced tolerance to SA. The progeny of candidate SP62-03 was further tested for SA tolerance and confirmed to be positive. SP62-03 was sequenced and its transgene matched ME08052.

Multiple events of ME08052 showed significantly better tolerance to SA than wild-type Ws. In a prevalidation assay, five events of ME08052 (T₂) together with wild-type controls were tested on SA plates for oxidative stress tolerance. Four events, -02, -03, 04 and -05, showed better growth on SA plates as compared to the control; Event -02 and -05 displayed especially strong tolerance phenotypes. The selected positive events were further validated in T₂ and T₃ generations.

T₂ and T₃ validation assays confirmed the SA stress tolerance in ME08052. To confirm that the transgene caused enhanced SA tolerance, T₂ and T₃ generations of the two positive events, ME08052-02 and -05, were further validated. The transgenic plants were compared to the internal non-transgenic plants, as well as external controls that were grown on the same SA plate. For each event by generation, two independent replicates (36 seedlings each) were analyzed. For each plant, seedling area was measured to reflect the growth rate under SA-induced oxidative stress (100 μM SA). As shown in Table 7, the T₂ transgenic plants of ME08052-02 and -05 grew significantly larger than pooled non-transgenic plants and the increases were 37.07 and 46.37%, respectively. The differences were repeated in T₃ generation. As compared to the pooled non-transgenic controls, the seedling areas of T₃ ME08052-02 and -05 plants increased 54.21 and 36.08%, respectively. These results indicated that the enhanced SA stress tolerance is mediated by the transgene.

TABLE 7
Validation assay of ME08052 on SA tolerance in two generations.

| | Seedling area | | | Seedling area | | | | |
|---|---|---|---|---|---|---|---|---|
| | of | | | of pooled | | | | |
| | transgenics | | | non-transgenics | | | t-test | Increase |
| ME Events | Avg | SE | N | Avg | SE | N | (p-value) | (%) |
| ME08052- | 0.62 | 0.048 | 38 | 0.45 | 0.054 | 33 | 0.0116 | 37.07% |
| 02T₂ | | | | | | | | |
| ME08052- | 0.78 | 0.040 | 39 | 0.53 | 0.064 | 31 | 0.0008 | 46.37% |
| 05T₂ | | | | | | | | |
| ME08052- | 1.15 | 0.056 | 42 | 0.75 | 0.094 | 30 | 0.0002 | 54.21% |
| 02T₃ | | | | | | | | |
| ME08052- | 0.98 | 0.069 | 42 | 0.72 | 0.081 | 29 | 0.0085 | 36.08% |
| 05T₃ | | | | | | | | |

Events ME08052-02 and -05 segregated for Finale™ resistance in the T₂ generation. Both segregated 3:1 (R:S) for Finale™, suggesting that there is only one copy of the transgene in each event. Events -02 and -05 of ME08052 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 11

### ME02927 (Ceres Clone 26652; SEQ ID No. 239)

ME02927 showed enhanced tolerance to oxidative stress induced by L-arginine ME02927 was identified from a superpool screen for L-arginine tolerance. Superpool 20 was screened for plants that showed enhanced tolerance to L-arginine. The progeny of the candidates were tested for L-arginine tolerance in a small square format. The selected tolerant candidates were sequenced and one of them matched the transgene sequence in ME02927.

Prevalidation revealed two positive events of ME02927 in L-arginine tolerance. Four T₂ events of ME02927 were compared to wild-type Ws for L-arginine tolerance in a small square format. Three events (ME02927-03 and -04) showed visually better growth under L-arginine stress as compared to Ws. The positive events were selected for further validation assay in T₂ and T₃ generations.

ME02927 showed significantly better tolerance to L-arginine than wild-type Ws. ME02927-03 and -04 T₂ plants were assayed in a validation configuration. The transgenic plants were compared to the internal non-transgenic plants as well as external controls that were grown on the same L-arginine plate. For each event, two independent replicates (36 seedlings each) were analyzed. Seedling area was measured for each plant. As shown in Table 8, the differences between transgenic and pooled non-transgenic plants are significant. These results indicate that the enhanced L-arginine stress tolerance is mediated by the transgene.

To confirm that the transgene caused the enhanced L-arginine tolerance, T₃ lines of ME02927-03 and -04 were further validated. As shown in Table 8, transgenic plants of ME02927-03-01, -03-02, -03-03 and -04-02 showed better growth than pooled non-transgenic plants under L-arginine-induced stress. These results confirmed that the observed better L-arginine stress tolerance in ME02927 is mediated by the transgene.

TABLE 8
Validation assay of ME02927 on arginine tolerance in two generations.

| | Seedling area | | | Seedling area of | | | | |
|---|---|---|---|---|---|---|---|---|
| | of | | | pooled non- | | | t-test | |
| ME | transgenics | | | transgenics | | | (p- | Increase |
| Events | Avg | SE | N | Avg | SE | N | value) | (%) |
| ME02927- | 1.224 | 0.053 | 39 | 0.873 | 0.098 | 32 | 1.17E−03 | 40.30% |
| 03T₂ | | | | | | | | |
| ME02927- | 4.869 | 0.199 | 40 | 3.910 | 0.382 | 32 | 0.0146 | 24.52% |
| 03T₃-01 | | | | | | | | |
| ME02927- | 4.191 | 0.200 | 33 | 3.216 | 0.311 | 38 | 0.0052 | 30.33% |
| 03T₃-02 | | | | | | | | |
| ME02927- | 4.299 | 0.274 | 28 | 3.242 | 0.337 | 43 | 0.0088 | 32.59% |
| 03T₃-03 | | | | | | | | |
| ME02927- | 0.775 | 0.051 | 43 | 0.372 | 0.080 | 25 | 3.42E−05 | 108.14% |
| 04T₂ | | | | | | | | |
| ME02927- | 3.661 | 0.273 | 33 | 2.404 | 0.323 | 36 | 0.0021 | 52.27% |
| 04T₃-02 | | | | | | | | |

Events ME02927-03 and -04 segregated for Finale™ resistance in the T₂ generation. Both segregate 3:1 (R:S) for Finale™, suggesting that there is only one copy of the transgene in each event. Events WO2927-03 and -04 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 12

### ME11932 (Ceres Clone At2g25920; SEQ ID No. 892)

ME11932 showed enhanced tolerance to oxidative stress induced by salicylic acid (SA) in Events -03 and -05 of both T₂ and T₃ generation. ME11932 was identified from superpool screen for SA tolerance. Superpool 90 was screened for transgenic plants that show enhanced tolerance to SA. The progeny of candidate SP90-11 was further tested for SA tolerance and confirmed to be positive. SP90-11 was sequenced and its transgene matched ME11932.

Multiple events of ME11932 showed significantly better tolerance to SA than wild-type Ws. In a prevalidation assay, five events of ME11932 (T₂) together with wild-type controls were tested on SA plates for oxidative stress tolerance. Two events, -03 and -05, showed significantly better growth on SA plates as compared to the control. The selected positive events were further validated in T₂ and T₃ generations.

T₂ and T₃ validation assays confirmed the SA stress tolerance in ME11932. To confirm that the transgene caused enhanced SA tolerance, T₂ and T₃ generations of the two positive events, ME11932-03 and -05, were further validated. The transgenic plants were compared to the internal non-transgenic plants, as well as external controls that were grown on the same SA plate. For each event by generation, two independent replicates (36 seedlings each) were analyzed. For each plant, seedling area was measured to reflect the growth rate under SA-induced oxidative stress (100 μM SA). As shown in Table 9, the T₂ transgenic plants of ME11932-03 and -05 grew significantly larger than pooled non-transgenic plants and the increases were 131.88 and 62.05%, respectively. The differences were repeated in T₃ generation. As compared to the pooled non-transgenic controls, the seedling areas of T₃ ME11932-03 and -05 plants increased 531.61 and 146.02%, respectively. These results indicated that the enhanced SA stress tolerance is mediated by the transgene.

TABLE 9
Validation assay of ME11932 on SA tolerance in two generations.

| | Seedling | | | Seedling area | | | | |
|---|---|---|---|---|---|---|---|---|
| | area of | | | of pooled | | | | |
| ME | transgenics | | | non-transgenics | | | t-test | Increase |
| Events | Avg | SE | N | Avg | SE | N | (p-value) | (%) |
| ME11932- | 0.90 | 0.065 | 42 | 0.39 | 0.119 | 16 | 0.0002 | 131.88% |
| 03T₂ | | | | | | | | |
| ME11932- | 0.54 | 0.072 | 39 | 0.33 | 0.099 | 23 | 0.0478 | 62.05% |
| 05T₂ | | | | | | | | |
| ME11932- | 0.48 | 0.066 | 25 | 0.08 | 0.026 | 8 | 1.47E−06 | 531.61% |
| 03T₃ | | | | | | | | |
| ME11932- | 0.44 | 0.073 | 32 | 0.18 | 0.056 | 26 | 0.0032 | 146.02% |
| 05T₃ | | | | | | | | |

Events ME11932-03 and -05 segregated for Finale™ resistance in the T₂ generation. ME11932-03 segregates 15:1 (R:S) for Finale™, whereas W11932-05 segregates 5:1, suggesting that there are two copies of the transgene in Event -03 and probably one copy in Event -05. Events -03 and -05 of ME11932 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 13

### ME24091 (Ceres Clone 106263; SEQ ID No. 922)

ME24091 showed enhanced tolerance to oxidative stress induced by salicylic acid (SA) in Events -05, -06, -07 and -09 of both T₂ and T₃ generations.

Multiple events of ME24091 showed significantly better tolerance to SA than wild-type Ws. In a prevalidation assay, ten events of ME24091 (T₂) together with wild-type controls were tested on SA plates for oxidative stress tolerance. All ten events showed better growth on SA plates as compared to the control. The selected positive events were further validated in T₂ and T₃ generations.

T₂ and T₃ validation assays confirmed the SA stress tolerance in ME24091. To confirm that the transgene caused enhanced SA tolerance, all ten T₂ events were validated. The transgenic plants were compared to the internal non-transgenic plants, as well as external controls that were grown on the same SA plate. For each event, two independent replicates (36 seedlings each) were analyzed. For each plant, seedling area was measured to reflect the growth rate under SA-induced oxidative stress (100 μM SA). As shown in Table 10, the T₂ transgenic plants of ME24091-02, -04, -05, -06, -07, -08, -09 and -10 grew significantly larger than pooled non-transgenic plants and the increases were 98.0, 141.5, 43.7, 148.1, 86.6, 88.9, 185.4 and 80.5%, respectively. The positive events were validated again in T₃ generation. The enhanced SA tolerance was confirmed in ME24091-05, -06, -07 and -07. As compared to the pooled non-transgenic controls, the seedling areas of T₃ plants in positive events increased 144.9, 118.2, 162.6 and 63.0%, respectively. These results indicated that the enhanced SA stress tolerance is mediated by the transgene.

TABLE 10
Validation assay of ME24091 on SA tolerance in two generations

| | Seedling | | | Seedling area | | | | |
|---|---|---|---|---|---|---|---|---|
| | area of | | | of pooled | | | | |
| ME | transgenics | | | non-transgenics | | | t-test | Increase |
| Events | Avg | SE | N | Avg | SE | N | (p-value) | (%) |
| ME24091- | 0.55 | 0.049 | 42 | 0.38 | 0.073 | 27 | 0.030 | 43.70% |
| 05T₂ | | | | | | | | |
| ME24091- | 0.71 | 0.049 | 39 | 0.29 | 0.064 | 23 | 1.14E−06 | 148.12% |
| 06T₂ | | | | | | | | |
| ME24091- | 0.69 | 0.054 | 37 | 0.37 | 0.074 | 25 | 0.000 | 86.59% |
| 07T₂ | | | | | | | | |
| ME24091- | 0.81 | 0.056 | 38 | 0.29 | 0.067 | 22 | 5.89E−08 | 185.42% |
| 09T₂ | | | | | | | | |
| ME24091- | 0.38 | 0.068 | 21 | 0.15 | 0.051 | 30 | 0.006 | 144.90% |
| 05T₃ | | | | | | | | |
| ME24091- | 0.49 | 0.073 | 19 | 0.22 | 0.063 | 22 | 0.005 | 118.19% |
| 06T₃ | | | | | | | | |
| ME24091- | 0.49 | 0.053 | 33 | 0.19 | 0.050 | 22 | 5.32E−05 | 162.61% |
| 07T₃ | | | | | | | | |
| ME24091- | 0.45 | 0.055 | 36 | 0.28 | 0.051 | 31 | 0.012 | 62.95% |
| 09T₃ | | | | | | | | |

Events of ME24091-05, -06, -07 and -09 segregated for Finale™ resistance in the T₂ generation. ME24091-05 segregates 3:1 (R:S) for Finale™, whereas ME24091-06, -07 and -09, segregate 1:1, 2:1 and 5:1, respectively, suggesting that there is likely one copy of the transgene in Events -05, -06, and -07, but two copies in Event -09. The inheritance of the transgene appears to be affected by integration site. Events -05, -06 and -07 of ME24091 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 14

### ME07302 (Ceres Clone 118036; SEQ ID No. 693)

ME07302 showed enhanced tolerance to oxidative stress induced by L-arginine Wild-type Ws seedlings showed stunted growth on plates containing 10 mM L-arginine, whereas the transgenic plants showed significantly better growth. ME07302 was identified from a superpool screen for L-arginine tolerance. Superpool 57 was screened for plants that showed enhanced tolerance to L-arginine. The progeny of the candidates were tested for L-arginine tolerance in a small square format. The selected tolerant candidates were sequenced and one of them matched the transgene sequence in ME07302.

Prevalidation revealed two positive events of ME07302 for L-arginine tolerance. Five T₂ events of ME07302 were compared to wild-type Ws for L-arginine tolerance in a small square format. Two events (ME07302-02, and -04) showed visually better growth under L-arginine stress as compared to Ws. The positive events were selected for further validation assay in T₂ and T₃ generations.

The functional confirmation of the transgene in L-arginine-induced stress tolerance. To confirm that the transgene caused the enhanced L-arginine tolerance, T₃ lines of ME07302-02 and -04 were further validated. As shown in Table 11, transgenic plants of all tested lines showed better growth than pooled non-transgenic plants under L-arginine-induced stress. These results confirmed that the observed better L-arginine stress tolerance in ME07302 is mediated by the transgene.

TABLE 11
Validation assay of ME07302 for L-arginine tolerance in two generations.

| | Seedling | | | Seedling area of | | | | |
|---|---|---|---|---|---|---|---|---|
| | area of | | | pooled non- | | | | |
| ME | transgenics | | | transgenics | | | t-test | Increase |
| Events | Avg | SE | N | Avg | SE | N | (p-value) | (%) |
| ME07302- | 0.781 | 0.026 | 51 | 0.480 | 0.073 | 21 | 0.000119 | 162.91% |
| 02T₂ | | | | | | | | |
| ME07302- | 0.597 | 0.014 | 54 | 0.433 | 0.060 | 18 | 0.004764 | 137.95% |
| 04T₂ | | | | | | | | |
| ME07302- | 4.050 | 0.175 | 36 | 1.800 | 0.297 | 36 | 4.45E−09 | 225.05% |
| 02T₃ | | | | | | | | |
| ME07302- | 4.603 | 0.154 | 54 | 2.481 | 0.423 | 17 | 6.03E−06 | 185.56% |
| 04T₃ | | | | | | | | |

Events ME07302-02 and -04 segregated for Finale™ resistance in the T₂ generation. Both segregate 3:1 (R:S) for Finale™, suggesting that there is only one copy of the transgene in each event. Events ME07302-02 and -04 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 15

### ME03365 (Ceres Clone 22538; SEQ ID No. 11)

ME03365 showed enhanced stress tolerance to high salt. ME03365 was identified from a superpool screen for salt stress tolerance. Superpool 22 was screened for transgenic plants that showed enhanced tolerance to high salt stress. One of the candidate plants was sequenced and the transgene sequence matched ME03365. Two events of ME03365 showed significantly better tolerance to high salt stress than wild-type Ws. Three T₂ events (-03, -04, -05) together with wild-type controls were tested on salt plates for tolerance. Two events (ME03365-03 and -04) showed obviously better growth on salt plates compared to the controls.

To confirm that the transgene caused enhanced salt stress tolerance, the transgenic plants were compared to the internal non-transgenic plants, as well as external controls that were grown on the same salt plate. Plants from T₂ and T₃ generations of ME03365-03 and -04 were tested further for salt tolerance. For each event by generation, two independent replicates (36 seedlings each) were analyzed. As shown in Table 12, the SGI value of transgenic plants increased by 62.5 and 41.0%, respectively, as compared to the pooled non-transgenic plants in T₂ generation.

In the T₃ generation, three individual lines of each event were tested. Five of them showed significantly better tolerance to high salt stress than non-transgenic controls. The SGI value of T₃ positive lines increased by 55.1, 93.9, 27.3, 324.8, 182.0 and 81.8%, respectively, as compared to the pooled non-transgenic controls. The differences between transgenic and non-transgenic plants are significant in ME03365-03 and -04 (T₂ generation) and ME03365-03-01, -03-02, -04-01, -04-02 and -04-03 (T₃ generation).

TABLE 12
Results of ME03365 on salt tolerance assay in two generations.

| | | | | SGI of pooled | | | | | |
|---|---|---|---|---|---|---|---|---|---|
| | SGI* of | | | non- | | | | | |
| | transgenics | | | transgenics | | | t-Test | | SGI |
| ME Events | Avg | SE | N | Avg | SE | N | t-value | t_{0.05} | increase |
| ME03365-03T₂ | 2.734 | 0.313 | 29 | 1.682 | 0.193 | 35 | 2.860 | 1.67 | 62.51% |
| ME03365-03T₃-01 | 3.565 | 0.241 | 20 | 2.298 | 0.260 | 24 | 3.575 | 1.68 | 55.14% |
| ME03365-03T₃-02 | 2.580 | 0.495 | 10 | 1.331 | 0.154 | 39 | 2.409 | 1.68 | 93.88% |
| ME03365-03T₃-03 | 2.332 | 0.260 | 26 | 1.831 | 0.322 | 24 | 1.209 | 1.68 | 27.30% |
| ME03365-04T₂ | 3.201 | 0.288 | 22 | 2.270 | 0.227 | 42 | 2.538 | 1.67 | 41.03% |
| ME03365-04T₃-01 | 2.933 | 0.404 | 15 | 0.690 | 0.129 | 41 | 5.288 | 1.68 | 324.76% |
| ME03365-04T₃-02 | 2.464 | 0.589 | 4 | 0.874 | 0.188 | 24 | 2.573 | 1.71 | 181.98% |
| ME03365-04T₃-03 | 3.065 | 0.344 | 23 | 1.686 | 0.241 | 43 | 3.282 | 1.67 | 81.75% |

*SGI (Salt Growth Index) = seedling area × Fv/Fm (photosynthesis efficiency)

Events ME03365-03 segregated for Finale™ in the T₂ generation. Both events segregated ˜1:1 for Finale™, suggesting that there is only one copy of the transgene with segregation distortion toward non-transgenics. Events -03 and -04 of ME03365 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 16

### ME08322 (Ceres Clone 560692; SEQ ID No. 102)

ME08322 showed enhanced stress tolerance to high salt. ME08322 was identified from a superpool screen for salt stress tolerance. Superpool 64 was screened for transgenic plants that show enhanced tolerance to high salt stress. One of the candidate plants was sequenced and the transgene sequence matches ME08322. Two events of ME08322 showed significantly better tolerance to high salt stress than wild-type Ws. Five T₂ events (-01 to -05) together with wild-type controls were tested on salt plates for tolerance. Two events (ME08322-01 and -05) showed obviously better growth on salt plates as compared to the control.

Functional confirmation of the transgene in salt stress tolerance. To confirm that the transgene caused enhanced salt stress tolerance, the transgenic plants were compared to the internal non-transgenic plants, as well as external controls that were grown on the same salt plate. Plants from T₂ and T₃ generations of ME08322-01 and -05 were tested further for salt tolerance. For each event by generation, two independent replicates (36 seedlings each) were analyzed. As shown in Table 13, the SGI value of transgenic plants increased by 24.3 and 40.6%, respectively, as compared to the pooled non-transgenic plants in T₂ generation.

In the T₃ generation, three individual lines of each event were tested. All of them showed significantly better tolerance to high salt stress than non-transgenic controls. The SGI value of T₃ positive lines increased by 45.3, 39.5, 65.0, 31.5, 29.4 and 177.7%, respectively, as compared to the pooled non-transgenic controls. The differences between transgenic and non-transgenic plants are significant in ME08322-01 and -05 (T₂ generation) and ME08322-01-01, -01-02, -01-03, -05-01, -05-02 and -05-03 (T₃ generation).

TABLE 13
Results of ME08322 on salt tolerance assay in two generations.

| | | | | SGI of pooled | | | | |
|---|---|---|---|---|---|---|---|---|
| | SGI* of | | | non- | | | | |
| ME | transgenics | | | transgenics | | | t-Test | SGI |
| Events | Avg | SE | N | Avg | SE | N | P-value | increase |
| ME08322- | 3.81 | 0.264 | 39 | 3.07 | 0.363 | 32 | 0.051 | 24.30% |
| 01T₂ | | | | | | | | |
| ME08322- | 3.99 | 0.315 | 32 | 2.84 | 0.371 | 32 | 0.010 | 40.58% |
| 05T₂ | | | | | | | | |
| ME08322- | 3.69 | 0.358 | 25 | 2.54 | 0.390 | 27 | 0.017 | 45.34% |
| 01T₂-01 | | | | | | | | |
| ME08322- | 2.97 | 0.325 | 27 | 2.13 | 0.304 | 25 | 0.032 | 39.46% |
| 01T₂-02 | | | | | | | | |
| ME08322- | 2.95 | 0.267 | 41 | 1.79 | 0.265 | 25 | 0.001 | 64.95% |
| 01T₂-03 | | | | | | | | |
| ME08322- | 2.87 | 0.208 | 42 | 2.18 | 0.303 | 30 | 0.033 | 31.49% |
| 05T₃-01 | | | | | | | | |
| ME08322- | 2.98 | 0.243 | 40 | 2.30 | 0.278 | 32 | 0.036 | 29.35% |
| 05T₃-02 | | | | | | | | |
| ME08322- | 3.62 | 0.191 | 48 | 1.30 | 0.197 | 24 | 1.42E−12 | 177.69% |
| 05T₃-03 | | | | | | | | |

*SGI (Salt Growth Index) = seedling area × Fv/Fm (photosynthesis efficiency)

Events ME08322-01 and -05 segregated for Finale™ resistance in the T₂ generation. Both events segregated 3:1 for Finale™, suggesting that there is only one copy of the transgene. Events -01 and -05 of ME08322 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 17

### ME08655 (Ceres Clone 547495; SEQ ID No. 32)

ME08655 showed enhanced stress tolerance to high salt. ME08655 was identified from a superpool screen for salt stress tolerance. Superpool 67 was screened for transgenic plants that show enhanced tolerance to high salt stress. One of the candidate plants was sequenced and the transgene sequence matches ME08655.

Three events of ME08655 showed significantly better tolerance to high salt stress than wild-type Ws. Five T₂ events (-01 to -05) together with wild-type controls were tested on salt plates for tolerance. Three events (ME08655-01, -02 and -05) showed obviously better growth on salt plates as compared to the control.

Functional confirmation of the transgene in salt stress tolerance. To confirm that the transgene caused enhanced salt stress tolerance, the transgenic plants were compared to the internal non-transgenic plants, as well as external controls that were grown on the same salt plate. Plants from T₂ generation of ME08655-01, -02 and -05 were validated for salt tolerance. For each event, two independent replicates (36 seedlings each) were analyzed. As shown in Table 14, the SGI value of transgenic plants increased by 27.7, 25.7 and 48.3%, respectively, as compared to the pooled non-transgenic plants in T₂ generation. The differences were significant for ME08655-02 and -05, but not for -01. The two positive events were further validated in the T₃ generation. Three individual lines of each event were tested. Four of them showed significantly better tolerance to high salt stress than non-transgenic controls. The SGI value of T₃ positive lines increased by 20.7, 29.0, 18.2 and 37.1%, respectively, as compared to the pooled non-transgenic controls.

TABLE 14
Results of ME08655 on salt tolerance assay in two generations.

| | | | | SGI of pooled | | | | |
|---|---|---|---|---|---|---|---|---|
| | SGI* of | | | non- | | | | |
| | transgenics | | | transgenics | | | t-Test | SGI |
| ME Events | Avg | SE | N | Avg | SE | N | p-value | increase |
| ME08655-02T₂ | 3.80 | 0.292 | 26 | 3.02 | 0.313 | 34 | 0.037 | 25.71% |
| ME08655- | 3.80 | 0.246 | 38 | 3.88 | 0.270 | 32 | 0.415 | −2.03% |
| 02T₃-01 | | | | | | | | |
| ME08655- | 3.75 | 0.209 | 32 | 3.49 | 0.201 | 39 | 0.179 | 07.70% |
| 02T₃-02 | | | | | | | | |
| ME08655- | 3.79 | 0.197 | 39 | 3.14 | 0.260 | 32 | 0.025 | 20.73% |
| 02T₃-03 | | | | | | | | |
| ME08655-05T₂ | 4.22 | 0.359 | 31 | 2.84 | 0.338 | 30 | 0.004 | 48.43% |
| ME08655- | 3.90 | 0.326 | 31 | 3.02 | 0.262 | 40 | 0.020 | 29.01% |
| 05T₃-01 | | | | | | | | |
| ME08655- | 3.74 | 0.241 | 34 | 3.17 | 0.249 | 35 | 0.051 | 18.19% |
| 05T₃-02 | | | | | | | | |
| ME08655- | 3.47 | 0.238 | 32 | 2.53 | 0.226 | 37 | 0.003 | 37.14% |
| 05T₃-03 | | | | | | | | |

*SGI (Salt Growth Index) = seedling area × Fv/Fm (photosynthesis efficiency)

Events ME08655-02 and -05 segregated for Finale™ resistance in the T₂ generation. ME08655-02 segregated ˜2:1 for Finale™, and ME08655-05 1:1, suggesting that there is likely one copy of the transgene with a segregation distortion towards non-transgenics. Events -02 and -05 of ME08655 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 18

### ME10409 (Ceres Clone At3g45100; SEQ ID No. 138)

ME10409 shows enhanced stress tolerance to high salt. ME10409 was identified from a superpool screen for salt stress tolerance. Superpool 77 was screened for transgenic plants that show enhanced tolerance to high salt stress. One of the candidate plants was sequenced and the transgene sequence matches ME10409.

Three events of ME10409 showed significantly better tolerance to high salt stress than wild-type Ws. Five T₂ events (-01 to -05) together with wild-type controls were tested on salt plates for tolerance. Three events (ME10409-01, -02 and -04) showed obviously better growth on salt plates as compared to the control.

Functional confirmation of the transgene in salt stress tolerance. To confirm that the transgene caused enhanced salt stress tolerance, the transgenic plants were compared to the internal non-transgenic plants, as well as external controls that were grown on the same salt plate. Plants of ME10409-01 and -04 from T₂ and T₃ generations were validated for salt tolerance. ME10409-02 was not tested because the seeds were unavailable. In the validation assay for each event by generation, two independent replicates (36 seedlings each) were analyzed. As shown in Table 15, the SGI value of transgenic plants increased by 50.3 and 71.1%, respectively, as compared to the pooled non-transgenic plants in T₂ generation. The differences were significant for ME10409-01 and -04. In T₃ generation, three individual lines of each event were tested. Four of them showed significantly better tolerance to high salt stress than non-transgenic controls. The SGI value of T₃ positive lines increased by 107.1, 50.0, 21.0 and 36.8%, respectively, as compared to the pooled non-transgenic controls. The differences between transgenic and non-transgenic plants in ME10409-01-01 and -01-03 and ME10409-04-02 and -04-03 were significant.

TABLE 15
Results of ME10409 on salt tolerance assay in two generations.

| | | | | SGI of pooled | | | | | |
|---|---|---|---|---|---|---|---|---|---|
| | SGI* of | | | non- | | | | | |
| | transgenics | | | transgenics | | | t-Test | | SGI |
| ME Events | Avg | SE | N | Avg | SE | N | P-value | P0.05 | increase |
| ME10409-01T₂ | 2.796 | 0.266 | 33 | 1.861 | 0.187 | 39 | 2.88 | 1.67 | 50.28% |
| ME10409-01T₃-01 | 4.473 | 0.300 | 35 | 2.159 | 0.298 | 30 | 5.47 | 1.67 | 107.12% |
| ME10409-01T₃-02 | 4.234 | 0.183 | 31 | 3.618 | 0.290 | 36 | 1.80 | 1.67 | 17.03% |
| ME10409-01T₃-03 | 3.478 | 0.215 | 39 | 2.319 | 0.265 | 31 | 3.40 | 1.67 | 49.99% |
| ME10409-04T₂ | 2.716 | 0.286 | 29 | 1.588 | 0.173 | 38 | 3.38 | 1.67 | 71.06% |
| ME10409-04T₃-01 | 4.164 | 0.270 | 32 | 3.544 | 0.365 | 34 | 1.36 | 1.67 | 17.49% |
| ME10409-04T₃-02 | 4.359 | 0.360 | 20 | 3.603 | 0.273 | 42 | 1.68 | 1.67 | 20.99% |
| ME10409-04T₃-03 | 4.117 | 0.323 | 24 | 3.010 | 0.340 | 42 | 2.36 | 1.67 | 36.76% |

*SGI (Salt Growth Index) = seedling area × Fv/Fm (photosynthesis efficiency)

Events ME10409-01 segregated for Finale™ in the T₂ generation. W10409-01 segregated ˜2:1 and ME10409-04 1:1 for Finale™ indicating some segregation distortion. Events -01 and -04 of ME10409 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 19

### ME17768 (Ceres Clone At4g00150; SEQ ID No. 149)

ME17768 showed enhanced stress tolerance to high salt. ME17768 was identified from a superpool screen for salt stress tolerance. Superpool 114 was screened for transgenic plants that show enhanced tolerance to high salt stress. One of the candidate plants was sequenced and the transgene sequence matched ME17768.

Three events of ME17768 showed better tolerance to high salt stress than wild-type Ws. Four T₂ events (-01 to -04) together with wild-type controls were tested on salt plates for tolerance. Three events (ME17768-02, -03 and -04) showed obviously better growth on salt plates as compared to the control.

Functional confirmation of the transgene in salt stress tolerance. To confirm that the transgene caused enhanced salt stress tolerance, the transgenic plants were compared to the internal non-transgenic plants, as well as external controls that were grown on the same salt plate. Plants from T₂ generation of ME17768-02, -03 and -04 validated for salt tolerance. For each event, two independent replicates (36 seedlings each) were analyzed. As shown in Table 16, the SGI value of transgenic plants increased by 325.1, 67.0 and 79.7% in ME17768-02, -03 and -04, respectively, as compared to the pooled non-transgenic plants in T₂ generation.

In T₃ generation, plants of ME17768-02 and -03 were further validated. Both showed significantly better tolerance to high salt stress than non-transgenic controls. The SGI value increased by 144.7 and 21.6%, respectively, as compared to the pooled non-transgenic controls. The differences between transgenic and non-transgenic plants are significant in all validated events.

TABLE 16
Results of ME17768 on salt tolerance assay in two generations.

| | | | | SGI of pooled | | | | |
|---|---|---|---|---|---|---|---|---|
| | SGI* of | | | non- | | | | |
| ME | transgenics | | | transgenics | | | t-Test | SGI |
| Events | Avg | SE | N | Avg | SE | N | P-value | increase |
| ME17768- | 1.02 | 0.188 | 10 | 0.24 | 0.144 | 7 | 0.002 | 325.14% |
| 02T₂ | | | | | | | | |
| ME17768- | 3.84 | 0.192 | 36 | 1.57 | 0.314 | 29 | 2.67E−08 | 144.70% |
| 02T₃ | | | | | | | | |
| ME17768- | 0.98 | 0.094 | 15 | 0.59 | 0.119 | 8 | 0.008 | 67.04% |
| 03T₂ | | | | | | | | |
| ME17768- | 3.60 | 0.202 | 42 | 2.96 | 0.297 | 30 | 0.040 | 21.60% |
| 03T₃ | | | | | | | | |

*SGI (Salt Growth Index) = seedling area × Fv/Fm (photosynthesis efficiency)

Events ME17768-02 and -03 segregated for Finale™ resistance in the T₂ generation. ME17768-02 segregated 8:1 for Finale™, suggesting that there are likely two copies of the transgene in this event. ME17768-03 segregated 3:1 for Finale™, suggesting that there is only one copy of the transgene in the event. Events -02 and -03 of ME17768 exhibited no statistically relevant negative phenotypes compared to the empty vector control SR00559.

There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 20

### ME12933 (Ceres Clone 210210; SEQ ID No. 2)

ME12933 showed enhanced stress tolerance to high salt. ME12933 was identified from a superpool screen for salt stress tolerance. Superpool 97 was screened for transgenic plants that show enhanced tolerance to high salt stress. One of the candidate plants was sequenced and the transgene sequence matches ME12933.

Two events of ME12933 showed significantly better tolerance to high salt stress than wild-type Ws. Five T₂ events (-01 to -05) together with wild-type controls were tested on salt plates for tolerance. Two events (ME12933-01 and -05) showed obviously better growth on salt plates as compared to the control.

Functional confirmation of the transgene in salt stress tolerance. To confirm that the transgene caused enhanced salt stress tolerance, the transgenic plants were compared to the internal non-transgenic plants, as well as external controls that were grown on the same salt plate. Plants from T₂ and T₃ generations of ME12933-01 and -05 were tested further for salt tolerance. For each event by generation, two independent replicates (36 seedlings each) were analyzed. As shown in Table 17, the SGI value of transgenic plants increased by 78.6 and 62.2%, respectively, as compared to the pooled non-transgenic plants in T₂ generation. In the T₃ generation, three individual lines of each event were tested. Four of them showed significantly better tolerance to high salt stress than non-transgenic controls. The SGI value of T₃ positive lines increased by 21.3, 47.6, 32.9 and 19.4%, respectively, as compared to the pooled non-transgenic controls. The differences between transgenic and non-transgenic plants are significant in ME12933-01 and -05 (T₂ generation) and ME12922-01-01, -01-03, -05-02 and -05-03 (T₃ generation).

TABLE 17
Results of ME12933 on salt tolerance assay in two generations.

| | | | | SGI of pooled | | | | | |
|---|---|---|---|---|---|---|---|---|---|
| | SGI* of | | | non- | | | t-Test | | |
| ME | transgenics | | | transgenics | | | t- | | SGI |
| Events | Avg | SE | N | Avg | SE | N | value | t_{0.05} | increase |
| ME12933- | 2.895 | 0.231 | 35 | 1.621 | 0.224 | 33 | 3.96 | 1.67 | 78.6% |
| 01 T₂ | | | | | | | | | |
| ME12933- | 2.830 | 0.235 | 33 | 1.745 | 0.223 | 38 | 3.35 | 1.67 | 62.2% |
| 05 T₂ | | | | | | | | | |
| ME12933- | 4.204 | 0.223 | 40 | 3.466 | 0.323 | 29 | 1.88 | 1.67 | 21.3% |
| 01-01 | | | | | | | | | |
| ME12933- | 4.069 | 0.258 | 37 | 3.432 | 0.340 | 26 | 1.49 | 1.67 | 18.6% |
| 01-02 | | | | | | | | | |
| ME12933- | 4.307 | 0.239 | 38 | 2.917 | 0.311 | 32 | 3.54 | 1.67 | 47.6% |
| 01-03 | | | | | | | | | |
| ME12933- | 3.747 | 0.225 | 38 | 3.845 | 0.335 | 33 | −0.24 | 1.67 | −2.6% |
| 05-01 | | | | | | | | | |
| ME12933- | 3.450 | 0.175 | 55 | 2.596 | 0.425 | 17 | 1.86 | 1.67 | 32.9% |
| 05-02 | | | | | | | | | |
| ME12933- | 3.935 | 0.259 | 35 | 3.295 | 0.256 | 32 | 1.76 | 1.67 | 19.4% |
| 05-03 | | | | | | | | | |

*SGI (Salt Growth Index) = seedling area × Fv/Fm (photosynthesis efficiency)

T₂ plants of ME12933-01 and -05 were tested on Finale™ plates for the number of copies of the transgene. ME12933-01 segregated 3:1 (R:S) for Finale™, suggesting that there is only one copy of the transgene in this event. ME12933-05 segregated 1:1 (R:S) for Finale™ indicating some segregation distortion.

Events -01 and -05 of ME12933 exhibited no statistically relevant negative phenotypes compared to the empty vector control SR00559. There was no detectable reduction in germination rate, no morphology/architecture difference as compared to non-transgenic plants was observed, and there were no observable or statistical differences between experimentals and controls for days to flowering, rosette area 7 days post-bolting, or fertility (silique number and seed fill).

### Example 21

### Determination of Functional Homologs by Reciprocal BLAST

A candidate sequence was considered a functional homolog of a reference sequence if the candidate and reference sequences encoded proteins having a similar function and/or activity. A process known as Reciprocal BLAST (Rivera et al., Proc. Natl. Acad. Sci. USA, 95:6239-6244 (1998)) was used to identify potential functional homolog sequences from databases consisting of all available public and proprietary peptide sequences, including NR from NCBI and peptide translations from Ceres clones.

Before starting a Reciprocal BLAST process, a specific reference polypeptide was searched against all peptides from its source species using BLAST in order to identify polypeptides having BLAST sequence identity of 80% or greater to the reference polypeptide and an alignment length of 85% or greater along the shorter sequence in the alignment. The reference polypeptide and any of the aforementioned identified polypeptides were designated as a cluster.

The BLASTP version 2.0 program from Washington University at Saint Louis, Mo., USA was used to determine BLAST sequence identity and E-value. The BLASTP version 2.0 program includes the following parameters: 1) an E-value cutoff of 1.0e-5; 2) a word size of 5; and 3) the -postsw option. The BLAST sequence identity was calculated based on the alignment of the first BLAST HSP (High-scoring Segment Pairs) of the identified potential functional homolog sequence with a specific reference polypeptide. The number of identically matched residues in the BLAST HSP alignment was divided by the HSP length, and then multiplied by 100 to get the BLAST sequence identity. The HSP length typically included gaps in the alignment, but in some cases gaps were excluded.

The main Reciprocal BLAST process consists of two rounds of BLAST searches; forward search and reverse search. In the forward search step, a reference polypeptide sequence, “polypeptide A,” from source species SA was BLASTed against all protein sequences from a species of interest. Top hits were determined using an E-value cutoff of 10⁻⁵ and a sequence identity cutoff of 35%. Among the top hits, the sequence having the lowest E-value was designated as the best hit, and considered a potential functional homolog or ortholog. Any other top hit that had a sequence identity of 80% or greater to the best hit or to the original reference polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog.

Functional homologs were identified by manual inspection of potential functional homolog sequences. Representative functional homologs for SEQ ID Nos. 2, 11, 102, 32, 138, 149, 441, 172, 764, 389, 316, 200, 697, 239, 892, 922, and 693 are shown in FIGS. 1-16 and in the Sequence Listing.

### Example 22

### Determination of Functional Homologs by Hidden Markov Models

Hidden Markov Models (HMMs) were generated by the program HMMER 2.3.2. To generate each HMM, the default HMMER 2.3.2 program parameters, configured for glocal alignments, were used.

An HMM was generated using the sequences shown in FIG. 1 as input. These sequences were input into the model and the HMM bit score for each sequence is shown in the Sequence Listing. Additional sequences were input into the model, and the HMM bit scores for the additional sequences are shown in the Sequence Listing. The results indicate that these additional sequences are functional homologs of SEQ ID NO: 2.

HMMs were also generated using the sequences shown in FIGS. 2-16 as input. These sequences were input into the respective models and the corresponding HMM bit score for each sequence is shown in the Sequence Listing. Additional sequences were input into the models, and the HMM bit scores for the additional sequences are shown in the Sequence Listing. The results indicate that these additional sequences are functional homologs of the groups in FIGS. 2-16.

### REFERENCES

The following references are cited in the Specification. Each of the references from the patent and periodical literature cited herein is hereby expressly incorporated in its entirety by such citation.
Zhang et al. (2004) Plant Physiol. 135:615.
Salomon et al. (1984) EMBO J. 3:141.
Herrera-Estrella et al. (1983) EMBO J. 2:987.
Escudero et al. (1996) Plant J. 10:355.
Ishida et al. (1996) Nature Biotechnology 14:745.
May et al. (1995) Bio/Technology 13:486)

Armaleo et al. (1990) Current Genetics 17:97.
Smith. T. F. and Waterman, M. S. (1981) Adv. App. Math. 2:482.
Needleman and Wunsch (1970) J. Mol. Biol. 48:443.
Pearson and Lipman (1988) Proc. Natl. Acad. Sci. (USA) 85: 2444.
Yamauchi et al. (1996) Plant Mol. Biol. 30:321-9.
Xu et al. (1995) Plant Mol. Biol. 27:237.
Yamamoto et al. (1991) Plant Cell 3:371.
P. Tijessen, “Hybridization with Nucleic Acid Probes” In Laboratory Techniques in Biochemistry and Molecular Biology, P. C. vand der Vliet, ed., c. 1993 by Elsevier, Amsterdam.
Bonner et al., (1973) J. Mol. Biol. 81:123.
Sambrook et al., Molecular Cloning: A Laboratory Manual, Second Edition, Cold Spring Harbor Laboratory Press, 1989, New York.
Shizuya et al. (1992) Proc. Natl. Acad. Sci. USA, 89: 8794-8797.
Hamilton et al. (1996) Proc. Natl. Acad. Sci. USA, 93: 9975-9979.
Burke et al. (1987) Science, 236:806-812.
Sternberg N. et al. (1990) Proc Natl Acad Sci U S A., 87:103-7.
Bradshaw et al. (1995) Nucl Acids Res, 23: 4850-4856.
Frischauf et al. (1983) J. Mol Biol, 170: 827-842.
Huynh et al., Glover N M (ed) DNA Cloning: A practical Approach, Vol. 1 Oxford: IRL Press (1985).
Walden et al. (1990) Mol Cell Biol 1: 175-194.
Vissenberg et al. (2005) Plant Cell Physiol 46:192.
Husebye et al. (2002) Plant Physiol 128:1180.
Plesch et al. (2001) Plant J 28:455.
Weising et al. (1988) Ann. Rev. Genet., 22:421.
Christou (1995) Euphytica, v. 85, n. 1-3:13-27.
Newell (2000)
Griesbach (1987) Plant Sci. 50:69-77.
Fromm et al. (1985) Proc. Natl. Acad. Sci. USA 82:5824.
Paszkowski et al. (1984) EMBO J. 3:2717.
Klein et al. (1987) Nature 327:773.
Willmitzer, L. (1993) Transgenic Plants. In: iotechnology, A Multi-Volume Comprehensive treatise (H. J. Rehm, G. Reed, A. Puler, P. Stadler, eds., Vol. 2, 627-659, VCH Weinheim-New York-Basel-Cambridge).
Crit. Rev. Plant. Sci. 4:1-46.
Fromm et al. (1990) Biotechnology 8:833-844.
Cho et al. (2000) Planta 210:195-204.
Brootghaerts et al. (2005) Nature 433:629-633.
Lincoln et al. (1998) Plant Mol. Biol. Rep. 16:1-4.
Lacomme et al. (2001), “Genetically Engineered Viruses” (C. J. A. Ring and E. D. Blair, Eds). Pp. 59-99, BIOS Scientific Publishers, Ltd. Oxford, UK.
Huh G H, Damsz B, Matsumoto T K, Reddy M P, Rus A M, Ibeas J I, Narasimhan M L, Bressan R A, Hasegawa P M, 2002, Salt causes ion disequilibrium-induced programmed cell death in yeast and plants. Plant J 29(5):649-59.
Kang D K, Li X M, Ochi K, Horinouchi S, 1999, Possible involvement of cAMP in aerial mycelium formation and secondary metabolism in Streptomyces griseus. Microbiology, 145 (Pt 5):1161-72.
Kerk D, Bulgrien J, Smith D W, Gribskov M, 2003, Arabidopsis proteins containing similarity to the universal stress protein domain of bacteria. Plant Physiol. 131(3):1209-19.
Zhu J K, 2001, Cell signaling under salt, water and cold stresses. Curr Opin Plant Biol. 4(5):401-6.
Susstrunk U, Pidoux J, Taubert S, Ullmann A, Thompson C J, 1998, Pleiotropic effects of cAMP on germination, antibiotic biosynthesis and morphological development in Streptomyces coelicolor. Mol Microbiol 30(1):33-46.
Davletova S, Schlauch K, Coutu J, Mittler R., 2005, The zinc-finger protein Zat12 plays a central role in reactive oxygen and abiotic stress signaling in Arabidopsis. Plant Physiol 139(2):847-56.
Fowler S G, Cook D, Thomashow M F., 2005, Low temperature induction of Arabidopsis CBF1, 2, and 3 is gated by the circadian clock. Plant Physiol 137(3):961-8.
Nachin L, Nannmark U, Nystom T (2005) Differential roles of the universal stress proteins of Escherichia coli in oxidative stress resistance, adhesion and motility J Bacteriol 187(18):6265-72.
Rizhsky L, Davletova S, Liang H, Mittler R, 2004, The zinc finger protein Zat12 is required for cytosolic ascorbate peroxidase 1 expression during oxidative stress in Arabidopsis. J Biol Chem. 19; 279(12):11736-43.—
Vogel J T, Zarka D G, Van Buskirk H A, Fowler S G, Thomashow M F, 2005, Roles of the CBF2 and ZAT12 transcription factors in configuring the low temperature transcriptome of Arabidopsis. Plant J. 41(2):195-211.
Sanchez-Barrena M J, Martinez-Ripoll M, Zhu J K, Albert A., 2005, The structure of the Arabidopsis thaliana SOS3: molecular mechanism of sensing calcium for salt stress response J Mol Biol. 345(5):1253-64.
Griffen, H. G, and Gasson, M. J. (1995) The Gene (aroK) Encoding Shikimate Kinase I from E. Coli. DNA Seq., 5(3):195-197.
Susstrunk et al. (1998) Mol Microbiol, 30(1):33-46
Kang et al. (1999) Microbiology, 145:1161-72.
Sauter M, Rzewuski G, Marwedel T, Lorbiecke R (2002) The novel ethylene-regulated gene OsUsp1 from rice encodes a member of a plant protein family related to prokaryotic universal stress proteins. J Exp Bot 53 (379):2325-31.
Kasuga et al. (1999) Nature Biotech 17: 287-291.
Rus et al. (2001) PNAS 98:14150-14155.
Shi et al. (2000) PNAS 97:6896-6901.
Apse et al. (1999) Science 285:1256-1258.
Zhang et al. (2001) PNAS 98:12832-12836.
Berthomieu et al. (2003) EMBO J 22:2004-2014.
Ren et al. (2005) Nat Genet. 37:1029-30
Davletova et al (2005) Plant Physiol. 139:847-56

1. A method of producing a plant having increased tolerance to salinity and/or increased tolerance to oxidative stress, said method comprising growing a plant cell comprising an exogenous nucleic acid, said exogenous nucleic acid comprising a regulatory region operably linked to a nucleotide sequence encoding a polypeptide, wherein the HMM bit score of the amino acid sequence of said polypeptide is greater than about 15, said HMM based on the amino acid sequences depicted in one of FIGS. 1 to 16, and wherein said plant has a difference in the level of salinity and/or oxidative stress tolerance as compared to the corresponding level of salinity and/or oxidative stress tolerance of a control plant that does not comprise said nucleic acid.

2. The method of claim 1, wherein the HMM bit score is greater than about 250 and wherein the polypeptide comprises XYPPX repeat domains.

3. The method of claim 1, wherein the HMM bit score is greater than about 65 based on the amino acid sequences depicted in FIG. 3 and wherein the polypeptide comprises an Cystatin domain having 70% percent or greater sequence identity to the Cystatin domain of any one of SEQ ID NO: 102, 103, 104, 106, 108, 110, 112, 114, 115, 116, 117, 118, 119, 120, 122, 124, 125, 127, 128, 129, 130, 132, 134, or 136.

4. The method of claim 1, wherein the HMM bit score is greater than about 175 based on the amino acid sequences depicted in FIG. 4 and wherein the polypeptide comprises an KOW domain having 70% percent or greater sequence identity to the KOW domain of any one of SEQ ID NO: 32, 33, 35, 37, 38, 40, 42, 44, 46, 48, 49, 51, 53, 54, 56, 58, 60, 62, 63, 64, 65, 66, 67, 68, 69, 71, 73, 75, 77, 79, 81, 83, 85, 87, 89, 91, 93, 95, 97, 98, 99, or 100 and wherein the polypeptide comprises an Ribosomal L27e protein family domain having 70% percent or greater sequence identity to the Ribosomal L27e protein family domain of any one of said SEQ ID NOs.

5. The method of claim 1, wherein the HMM bit score is greater than about 1095 based on the amino acid sequences depicted in FIG. 5 and wherein the polypeptide comprises an Glycosyl transferases group 1 domain having 70% percent or greater sequence identity to the Glycosyl transferases group 1 domain of any one of SEQ ID NO: 138, 140, 142, 144, 146, or 147, and wherein the polypeptide comprises an PIGA domain having 70% percent or greater sequence identity to the PIGA domain of any one of said SEQ ID NOs.

6. The method of claim 1, wherein the HMM bit score is greater than about 535 based on the amino acid sequences depicted in FIG. 6 and wherein the polypeptide comprises an GRAS domain having 70% percent or greater sequence identity to the GRAS domain of any one of SEQ ID NO: 149, 151, 153, 154, 155, 157, 159, 161, 162, 163, 164, 165, 167, or 169.

7. The method of claim 1, wherein the HMM bit score is greater than about 120 based on the amino acid sequences depicted in FIG. 7 and wherein the polypeptide comprises an Cytochrome P450 domain having 70% percent or greater sequence identity to the Cytochrome P450 domain of any one of SEQ ID NO: 441, 442, 444, 445, 446, 447, 449, 450, 451, 452, 453, 454, 456, 457, 458, 459, 460, 461, 462, 463, 464, 465, 467, 468, 469, 470, 471, 472, 473, 474, 475, 477, 479, 480, 481, 482, 484, 486, 487, 489, 490, 491, 492, 493, 494, 496, 498, 500, 502, 503, 504, 505, 506, 507, 508, 509, 511, 512, 513, 514, 516, 517, 518, 519, 520, 522, 523, 524, 525, 527, 529, 531, 532, 533, 534, 536, 537, 538, 539, 540, 541, 543, 544, 546, 547, 549, 550, 551, 552, 553, 555, 556, 557, 559, 560, 562, 563, 564, 565, 566, 567, 569, 571, 572, 574, 576, 577, 578, 580, 582, 583, 585, 586, 588, 590, 591, 592, 593, 594, 595, 597, 598, 600, 601, 603, 605, 606, 608, 609, 610, 611, 613, 614, 615, 616, 617, 618, 620, 622, 624, 625, 626, 627, 629, 631, 633, 635, 637, 639, 640, 641, 642, 643, 644, 646, 648, 650, 651, 653, 654, 655, 657, 659, 660, 661, 662, 663, 664, 665, 666, 668, 669, 671, 673, 675, 677, 678, 680, 682, 684, 686, 688, or 690.

8. The method of claim 1, wherein the HMM bit score is greater than about 180 based on the amino acid sequences depicted in FIG. 9 and wherein the polypeptide comprises an Cyclin, N-terminal domain having 70% percent or greater sequence identity to the Cyclin, N-terminal domain of any one of SEQ ID NO: 764, 765, 766, 767, 769, 770, 771, 773, 774, 775, 776, 777, 779, 780, 781, 782, 784, 785, 786, 788, 790, 792, 793, 795, 796, 797, 798, 801, 802, 804, 805, 808, 809, 810, 812, 813, 814, 815, 816, 817, 818, 819, 820, 822, 824, 825, 826, 827, 829, 832, 834, 836, 838, 840, 841, 843, 845, 846, 847, 848, 849, 850, 851, 852, 854, 855, 856, 857, 858, 860, 861, 862, 863, 864, 867, 870, 871, 872, 874, 876, 878, 880, 882, 884, 886, 888, or 890, and wherein the polypeptide comprises an Cyclin, C-terminal domain having 70% percent or greater sequence identity to the Cyclin, C-terminal domain of any one of said SEQ ID NOs.

9. The method of claim 1, wherein the HMM bit score is greater than about 110 based on the amino acid sequences depicted in FIG. 10 and wherein the polypeptide comprises an MATH domain having 70% percent or greater sequence identity to the MATH domain of any one of SEQ ID NO: 389, 392, 394, 396, 398, 400, 401, 402, 403, 405, 406, 407, 408, 410, 411, 412, 414, 416, 417, 418, 419, 421, 422, 424, 426, 428, 430, 431, 432, 433, 434, 436, 437, or 438.

10. The method of claim 1, wherein the HMM bit score is greater than about 75 based on the amino acid sequences depicted in FIG. 11 and wherein the polypeptide comprises an LSD1 zinc finger domain having 70% percent or greater sequence identity to the LSD1 zinc finger domain of any one of SEQ ID NO: 316, 318, 319, 320, 321, 323, 324, 331, 333, 335, 337, 339, 340, 344, 346, 348, 350, 351, 353, 355, 356, 357, 359, 361, 363, 365, 367, 369, 374, 375, 377, 378, 380, 382, 384, or 386.

11. The method of claim 1, wherein the HMM bit score is greater than about 50 based on the amino acid sequences depicted in FIG. 14 and wherein the polypeptide comprises an LSD1 zinc finger domain having 70% percent or greater sequence identity to the LSD1 zinc finger domain of any one of SEQ ID NO: 239, 241, 243, 245, 247, 249, 250, 251, 253, 255, 257, 259, 261, 262, 264, 266, 270, 271, 272, 273, 275, 279, 281, 282, 284, 286, 288, 290, 291, 292, 293, 294, 295, 297, 299, 301, 302, 304, 306, or 308.

12. The method of claim 1, wherein the HMM bit score is greater than about 70 based on the amino acid sequences depicted in FIG. 13 and wherein the polypeptide comprises an GLTP domain having 70% percent or greater sequence identity to the GLTP domain of any one of SEQ ID NO: 697, 699, 703, 706, 707, 709, 711, 712, 714, 718, 720, 722, 723, 726, 728, 730, 732, 733, 735, 737, 739, 740, 741, 742, 743, 745, 746, 747, 749, 751, 752, 753, 755, 757, 759, or 761.

13. The method of claim 1, wherein the HMM bit score is greater than about 25 based on the amino acid sequences depicted in FIG. 16 and wherein the polypeptide comprises an IQ calmodulin-binding motif domain having 70% percent or greater sequence identity to the IQ calmodulin-binding motif domain of any one of SEQ ID NO: 724, 806, 831, 866, 868, 869, 899, 922, 924, 926, 928, 930, 932, 934, 936, 938, 940, 942, 944, 946, 948, 950, 952, 954, 956, 958, 960, 962, 964, 966, 968, 970, 972, 974, 975, 976, 977, 978, 979, 981, 982, 983, 985, 987, 989, 991, 993, 994, 995, or 996.

14. A method of producing a plant, said method comprising growing a plant cell comprising an exogenous nucleic acid, said exogenous nucleic acid comprising a regulatory region operably linked to a nucleotide sequence encoding a polypeptide having 80 percent or greater sequence identity to an amino acid sequence selected from the group consisting of SEQ ID NO: 2, 4, 6, 7, 9, 11, 13, 15, 17, 19, 21, 23, 25, 27, 29, 30, 32, 33, 35, 37, 38, 40, 42, 44, 46, 48, 49, 51, 53, 54, 56, 58, 60, 62, 63, 64, 65, 66, 67, 68, 69, 71, 73, 75, 77, 79, 81, 83, 85, 87, 89, 91, 93, 95, 97, 98, 99, 100, 102, 103, 104, 106, 108, 110, 112, 114, 115, 116, 117, 118, 119, 120, 122, 124, 125, 127, 128, 129, 130, 132, 134, 136, 138, 140, 142, 144, 146, 147, 149, 151, 153, 154, 155, 157, 159, 161, 162, 163, 164, 165, 167, 169, 172, 174, 175, 178, 179, 180, 182, 184, 186, 187, 188, 189, 191, 192, 194, 196, 198, 200, 202, 204, 206, 208, 211, 217, 219, 223, 225, 226, 228, 230, 232, 233, 235, 237, 239, 241, 243, 245, 247, 249, 250, 251, 253, 255, 257, 259, 261, 262, 264, 266, 270, 271, 272, 273, 275, 279, 281, 282, 284, 286, 288, 290, 291, 292, 293, 294, 295, 297, 299, 301, 302, 304, 306, 308, 316, 318, 319, 320, 321, 323, 324, 331, 333, 335, 337, 339, 340, 344, 346, 348, 350, 351, 353, 355, 356, 357, 359, 361, 363, 365, 367, 369, 374, 375, 377, 378, 380, 382, 384, 386, 389, 392, 394, 396, 398, 400, 401, 402, 403, 405, 406, 407, 408, 410, 411, 412, 414, 416, 417, 418, 419, 421, 422, 424, 426, 428, 430, 431, 432, 433, 434, 436, 437, 438, 441, 442, 444, 445, 446, 447, 449, 450, 451, 452, 453, 454, 456, 457, 458, 459, 460, 461, 462, 463, 464, 465, 467, 468, 469, 470, 471, 472, 473, 474, 475, 477, 479, 480, 481, 482, 484, 486, 487, 489, 490, 491, 492, 493, 494, 496, 498, 500, 502, 503, 504, 505, 506, 507, 508, 509, 511, 512, 513, 514, 516, 517, 518, 519, 520, 522, 523, 524, 525, 527, 529, 531, 532, 533, 534, 536, 537, 538, 539, 540, 541, 543, 544, 546, 547, 549, 550, 551, 552, 553, 555, 556, 557, 559, 560, 562, 563, 564, 565, 566, 567, 569, 571, 572, 574, 576, 577, 578, 580, 582, 583, 585, 586, 588, 590, 591, 592, 593, 594, 595, 597, 598, 600, 601, 603, 605, 606, 608, 609, 610, 611, 613, 614, 615, 616, 617, 618, 620, 622, 624, 625, 626, 627, 629, 631, 633, 635, 637, 639, 640, 641, 642, 643, 644, 646, 648, 650, 651, 653, 654, 655, 657, 659, 660, 661, 662, 663, 664, 665, 666, 668, 669, 671, 673, 675, 677, 678, 680, 682, 684, 686, 688, 690, 693, 694, 697, 699, 703, 706, 707, 709, 711, 712, 714, 718, 720, 722, 723, 724, 726, 728, 730, 732, 733, 735, 737, 739, 740, 741, 742, 743, 745, 746, 747, 749, 751, 752, 753, 755, 757, 759, 761, 764, 765, 766, 767, 769, 770, 771, 773, 774, 775, 776, 777, 779, 780, 781, 782, 784, 785, 786, 788, 790, 792, 793, 795, 796, 797, 798, 801, 802, 804, 805, 808, 809, 810, 812, 813, 814, 815, 816, 817, 818, 819, 820, 822, 824, 825, 826, 827, 829, 831, 832, 832, 834, 836, 838, 840, 841, 843, 845, 846, 847, 848, 849, 850, 851, 852, 854, 855, 856, 857, 858, 860, 861, 862, 863, 864, 867, 867, 868, 869, 870, 871, 872, 874, 876, 878, 880, 882, 884, 886, 888, 890, 892, 894, 896, 898, 899, 901, 903, 905, 906, 907, 909, 911, 913, 914, 915, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 975, 976, 977, 978, 979, 981, 982, 983, 985, 987, 989, 991, 993, 994, 995, and 996, wherein a plant produced from said plant cell has a difference in the level of salinity and/or oxidative stress tolerance as compared to the corresponding level of salinity and/or oxidative stress tolerance of a control plant that does not comprise said nucleic acid.

15. The method of claim 14, wherein the HMM bit score of the amino acid sequence of said polypeptide is greater than about 15, said HMM based on the amino acid sequences depicted in any one of FIGS. 1 to 16.

16. A method of producing a plant, said method comprising growing a plant cell comprising an exogenous nucleic acid, said exogenous nucleic acid comprising a regulatory region operably linked to a nucleotide sequence having 80 percent or greater sequence identity to a fragment of a nucleotide sequence selected from the group consisting of SEQ ID NO: 1, 3, 5, 8, 10, 12, 14, 16, 18, 20, 22, 24, 26, 28, 31, 34, 36, 39, 41, 43, 45, 47, 50, 52, 55, 7, 59, 61, 70, 72, 74, 76, 78, 80, 82, 84, 86, 88, 90, 92, 94, 96, 101, 105, 107, 109, 111, 113, 121, 123, 126, 131, 133, 135, 137, 139, 141, 145, 148, 150, 152, 156, 158, 160, 166, 168, 170, 171, 173, 177, 181, 183, 185, 190, 193, 195, 197, 199, 201, 203, 205, 207, 210, 216, 218, 222, 224, 227, 229, 231, 234, 236, 238, 240, 242, 244, 246, 248, 252, 254, 256, 258, 260, 263, 265, 269, 274, 278, 280, 283, 285, 287, 289, 296, 298, 300, 303, 305, 307, 315, 317, 322, 330, 332, 334, 336, 338, 343, 345, 347, 349, 352, 354, 358, 360, 362, 364, 366, 368, 376, 379, 381, 383, 385, 387, 388, 391, 393, 395, 397, 399, 404, 409, 413, 415, 420, 423, 425, 427, 429, 435, 439, 440, 443, 448, 455, 466, 476, 478, 483, 485, 488, 495, 497, 499, 501, 510, 515, 521, 526, 528, 530, 535, 542, 545, 548, 554, 558, 561, 568, 570, 573, 575, 579, 581, 584, 587, 589, 596, 599, 602, 604, 607, 612, 619, 621, 623, 628, 630, 632, 634, 636, 638, 645, 647, 649, 652, 656, 658, 667, 670, 672, 674, 676, 679, 681, 683, 685, 687, 689, 691, 692, 695, 696, 698, 702, 708, 710, 713, 717, 719, 721, 725, 727, 729, 731, 734, 736, 738, 744, 748, 750, 754, 756, 758, 760, 762, 763, 768, 772, 778, 783, 787, 789, 791, 794, 799, 800, 803, 807, 811, 821, 823, 828, 830, 833, 835, 837, 839, 842, 844, 853, 859, 865, 873, 875, 877, 879, 881, 883, 885, 887, 889, 891, 893, 895, 897, 900, 902, 904, 908, 910, 912, 917, 919, 921, 923, 925, 927, 929, 931, 933, 935, 937, 939, 941, 943, 945, 947, 949, 951, 953, 955, 957, 959, 961, 963, 965, 967, 969, 971, 973, 980, 984, 986, 988, 990, and 992, wherein a plant produced from said plant cell has a difference in the level of salinity and/or oxidative stress tolerance as compared to the corresponding level of salinity and/or oxidative stress tolerance of a control plant that does not comprise said nucleic acid.

17. A method of modulating the level of salinity and/or oxidative stress tolerance in a plant, said method comprising introducing into a plant cell an exogenous nucleic acid, said exogenous nucleic acid comprising a regulatory region operably linked to a nucleotide sequence encoding a polypeptide, wherein the HMM bit score of the amino acid sequence of said polypeptide is greater than about 15, said HMM based on the amino acid sequences depicted in one of FIGS. 1 to 16, and wherein a plant produced from said plant cell has a difference in the level of salinity and/or oxidative stress tolerance as compared to the corresponding level of salinity and/or oxidative stress tolerance of a control plant that does not comprise said exogenous nucleic acid.

18. A method of modulating the level of salinity and/or oxidative stress tolerance in a plant, said method comprising introducing into a plant cell an exogenous nucleic acid, said exogenous nucleic acid comprising a regulatory region operably linked to a nucleotide sequence encoding a polypeptide having 80 percent or greater sequence identity to an amino acid sequence selected from the group consisting of SEQ ID NO: 2, 4, 6, 7, 9, 11, 13, 15, 17, 19, 21, 23, 25, 27, 29, 30, 32, 33, 35, 37, 38, 40, 42, 44, 46, 48, 49, 51, 53, 54, 56, 58, 60, 62, 63, 64, 65, 66, 67, 68, 69, 71, 73, 75, 77, 79, 81, 83, 85, 87, 89, 91, 93, 95, 97, 98, 99, 100, 102, 103, 104, 106, 108, 110, 112, 114, 115, 116, 117, 118, 119, 120, 122, 124, 125, 127, 128, 129, 130, 132, 134, 136, 138, 140, 142, 144, 146, 147, 149, 151, 153, 154, 155, 157, 159, 161, 162, 163, 164, 165, 167, 169, 172, 174, 175, 178, 179, 180, 182, 184, 186, 187, 188, 189, 191, 192, 194, 196, 198, 200, 202, 204, 206, 208, 211, 217, 219, 223, 225, 226, 228, 230, 232, 233, 235, 237, 239, 241, 243, 245, 247, 249, 250, 251, 253, 255, 257, 259, 261, 262, 264, 266, 270, 271, 272, 273, 275, 279, 281, 282, 284, 286, 288, 290, 291, 292, 293, 294, 295, 297, 299, 301, 302, 304, 306, 308, 316, 318, 319, 320, 321, 323, 324, 331, 333, 335, 337, 339, 340, 344, 346, 348, 350, 351, 353, 355, 356, 357, 359, 361, 363, 365, 367, 369, 374, 375, 377, 378, 380, 382, 384, 386, 389, 392, 394, 396, 398, 400, 401, 402, 403, 405, 406, 407, 408, 410, 411, 412, 414, 416, 417, 418, 419, 421, 422, 424, 426, 428, 430, 431, 432, 433, 434, 436, 437, 438, 441, 442, 444, 445, 446, 447, 449, 450, 451, 452, 453, 454, 456, 457, 458, 459, 460, 461, 462, 463, 464, 465, 467, 468, 469, 470, 471, 472, 473, 474, 475, 477, 479, 480, 481, 482, 484, 486, 487, 489, 490, 491, 492, 493, 494, 496, 498, 500, 502, 503, 504, 505, 506, 507, 508, 509, 511, 512, 513, 514, 516, 517, 518, 519, 520, 522, 523, 524, 525, 527, 529, 531, 532, 533, 534, 536, 537, 538, 539, 540, 541, 543, 544, 546, 547, 549, 550, 551, 552, 553, 555, 556, 557, 559, 560, 562, 563, 564, 565, 566, 567, 569, 571, 572, 574, 576, 577, 578, 580, 582, 583, 585, 586, 588, 590, 591, 592, 593, 594, 595, 597, 598, 600, 601, 603, 605, 606, 608, 609, 610, 611, 613, 614, 615, 616, 617, 618, 620, 622, 624, 625, 626, 627, 629, 631, 633, 635, 637, 639, 640, 641, 642, 643, 644, 646, 648, 650, 651, 653, 654, 655, 657, 659, 660, 661, 662, 663, 664, 665, 666, 668, 669, 671, 673, 675, 677, 678, 680, 682, 684, 686, 688, 690, 693, 694, 697, 699, 703, 706, 707, 709, 711, 712, 714, 718, 720, 722, 723, 724, 726, 728, 730, 732, 733, 735, 737, 739, 740, 741, 742, 743, 745, 746, 747, 749, 751, 752, 753, 755, 757, 759, 761, 764, 765, 766, 767, 769, 770, 771, 773, 774, 775, 776, 777, 779, 780, 781, 782, 784, 785, 786, 788, 790, 792, 793, 795, 796, 797, 798, 801, 802, 804, 805, 808, 809, 810, 812, 813, 814, 815, 816, 817, 818, 819, 820, 822, 824, 825, 826, 827, 829, 831, 832, 832, 834, 836, 838, 840, 841, 843, 845, 846, 847, 848, 849, 850, 851, 852, 854, 855, 856, 857, 858, 860, 861, 862, 863, 864, 867, 867, 868, 869, 870, 871, 872, 874, 876, 878, 880, 882, 884, 886, 888, 890, 892, 894, 896, 898, 899, 901, 903, 905, 906, 907, 909, 911, 913, 914, 915, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 975, 976, 977, 978, 979, 981, 982, 983, 985, 987, 989, 991, 993, 994, 995, and 996, wherein a plant produced from said plant cell has a difference in the level of salinity and/or oxidative stress tolerance as compared to the corresponding level of salinity and/or oxidative stress tolerance of a control plant that does not comprise said nucleic acid.

19. The method of any one of claims 1, 14, 17 or 18, wherein said polypeptide is selected from the group consisting of SEQ ID NO: 2, 11, 102, 32, 138, 149, 441, 172, 764, 389, 316, 200, 697, 239, 892, 922, and 693.

20. A method of modulating the level of salinity and/or oxidative stress tolerance in a plant, said method comprising introducing into a plant cell an exogenous nucleic acid, said exogenous nucleic acid comprising a regulatory region operably linked to a nucleotide sequence having 80 percent or greater sequence identity to a nucleotide sequence selected from the group consisting of SEQ ID NO: 1, 3, 5, 8, 10, 12, 14, 16, 18, 20, 22, 24, 26, 28, 31, 34, 36, 39, 41, 43, 45, 47, 50, 52, 55, 7, 59, 61, 70, 72, 74, 76, 78, 80, 82, 84, 86, 88, 90, 92, 94, 96, 101, 105, 107, 109, 111, 113, 121, 123, 126, 131, 133, 135, 137, 139, 141, 145, 148, 150, 152, 156, 158, 160, 166, 168, 170, 171, 173, 177, 181, 183, 185, 190, 193, 195, 197, 199, 201, 203, 205, 207, 210, 216, 218, 222, 224, 227, 229, 231, 234, 236, 238, 240, 242, 244, 246, 248, 252, 254, 256, 258, 260, 263, 265, 269, 274, 278, 280, 283, 285, 287, 289, 296, 298, 300, 303, 305, 307, 315, 317, 322, 330, 332, 334, 336, 338, 343, 345, 347, 349, 352, 354, 358, 360, 362, 364, 366, 368, 376, 379, 381, 383, 385, 387, 388, 391, 393, 395, 397, 399, 404, 409, 413, 415, 420, 423, 425, 427, 429, 435, 439, 440, 443, 448, 455, 466, 476, 478, 483, 485, 488, 495, 497, 499, 501, 510, 515, 521, 526, 528, 530, 535, 542, 545, 548, 554, 558, 561, 568, 570, 573, 575, 579, 581, 584, 587, 589, 596, 599, 602, 604, 607, 612, 619, 621, 623, 628, 630, 632, 634, 636, 638, 645, 647, 649, 652, 656, 658, 667, 670, 672, 674, 676, 679, 681, 683, 685, 687, 689, 691, 692, 695, 696, 698, 702, 708, 710, 713, 717, 719, 721, 725, 727, 729, 731, 734, 736, 738, 744, 748, 750, 754, 756, 758, 760, 762, 763, 768, 772, 778, 783, 787, 789, 791, 794, 799, 800, 803, 807, 811, 821, 823, 828, 830, 833, 835, 837, 839, 842, 844, 853, 859, 865, 873, 875, 877, 879, 881, 883, 885, 887, 889, 891, 893, 895, 897, 900, 902, 904, 908, 910, 912, 917, 919, 921, 923, 925, 927, 929, 931, 933, 935, 937, 939, 941, 943, 945, 947, 949, 951, 953, 955, 957, 959, 961, 963, 965, 967, 969, 971, 973, 980, 984, 986, 988, 990, and 992, or a fragment thereof, wherein a plant produced from said plant cell has a difference in the level of salinity and/or oxidative stress tolerance as compared to the corresponding level in of salinity and/or oxidative stress tolerance of a control plant that does not comprise said nucleic acid.

21. A plant cell comprising an exogenous nucleic acid said exogenous nucleic acid comprising a regulatory region operably linked to a nucleotide sequence encoding a polypeptide, wherein the HMM bit score of the amino acid sequence of said polypeptide is greater than about 15, said HMM based on the amino acid sequences depicted in one of FIGS. 1 to 16, and wherein said plant has a difference in the level of salinity and/or oxidative stress tolerance as compared to the corresponding level of salinity and/or oxidative stress tolerance of a control plant that does not comprise said nucleic acid.

22. A plant cell comprising an exogenous nucleic acid said exogenous nucleic acid comprising a regulatory region operably linked to a nucleotide sequence encoding a polypeptide having 80 percent or greater sequence identity to an amino acid sequence selected from the group consisting of SEQ ID NO: 2, 4, 6, 7, 9, 11, 13, 15, 17, 19, 21, 23, 25, 27, 29, 30, 32, 33, 35, 37, 38, 40, 42, 44, 46, 48, 49, 51, 53, 54, 56, 58, 60, 62, 63, 64, 65, 66, 67, 68, 69, 71, 73, 75, 77, 79, 81, 83, 85, 87, 89, 91, 93, 95, 97, 98, 99, 100, 102, 103, 104, 106, 108, 110, 112, 114, 115, 116, 117, 118, 119, 120, 122, 124, 125, 127, 128, 129, 130, 132, 134, 136, 138, 140, 142, 144, 146, 147, 149, 151, 153, 154, 155, 157, 159, 161, 162, 163, 164, 165, 167, 169, 172, 174, 175, 178, 179, 180, 182, 184, 186, 187, 188, 189, 191, 192, 194, 196, 198, 200, 202, 204, 206, 208, 211, 217, 219, 223, 225, 226, 228, 230, 232, 233, 235, 237, 239, 241, 243, 245, 247, 249, 250, 251, 253, 255, 257, 259, 261, 262, 264, 266, 270, 271, 272, 273, 275, 279, 281, 282, 284, 286, 288, 290, 291, 292, 293, 294, 295, 297, 299, 301, 302, 304, 306, 308, 316, 318, 319, 320, 321, 323, 324, 331, 333, 335, 337, 339, 340, 344, 346, 348, 350, 351, 353, 355, 356, 357, 359, 361, 363, 365, 367, 369, 374, 375, 377, 378, 380, 382, 384, 386, 389, 392, 394, 396, 398, 400, 401, 402, 403, 405, 406, 407, 408, 410, 411, 412, 414, 416, 417, 418, 419, 421, 422, 424, 426, 428, 430, 431, 432, 433, 434, 436, 437, 438, 441, 442, 444, 445, 446, 447, 449, 450, 451, 452, 453, 454, 456, 457, 458, 459, 460, 461, 462, 463, 464, 465, 467, 468, 469, 470, 471, 472, 473, 474, 475, 477, 479, 480, 481, 482, 484, 486, 487, 489, 490, 491, 492, 493, 494, 496, 498, 500, 502, 503, 504, 505, 506, 507, 508, 509, 511, 512, 513, 514, 516, 517, 518, 519, 520, 522, 523, 524, 525, 527, 529, 531, 532, 533, 534, 536, 537, 538, 539, 540, 541, 543, 544, 546, 547, 549, 550, 551, 552, 553, 555, 556, 557, 559, 560, 562, 563, 564, 565, 566, 567, 569, 571, 572, 574, 576, 577, 578, 580, 582, 583, 585, 586, 588, 590, 591, 592, 593, 594, 595, 597, 598, 600, 601, 603, 605, 606, 608, 609, 610, 611, 613, 614, 615, 616, 617, 618, 620, 622, 624, 625, 626, 627, 629, 631, 633, 635, 637, 639, 640, 641, 642, 643, 644, 646, 648, 650, 651, 653, 654, 655, 657, 659, 660, 661, 662, 663, 664, 665, 666, 668, 669, 671, 673, 675, 677, 678, 680, 682, 684, 686, 688, 690, 693, 694, 697, 699, 703, 706, 707, 709, 711, 712, 714, 718, 720, 722, 723, 724, 726, 728, 730, 732, 733, 735, 737, 739, 740, 741, 742, 743, 745, 746, 747, 749, 751, 752, 753, 755, 757, 759, 761, 764, 765, 766, 767, 769, 770, 771, 773, 774, 775, 776, 777, 779, 780, 781, 782, 784, 785, 786, 788, 790, 792, 793, 795, 796, 797, 798, 801, 802, 804, 805, 808, 809, 810, 812, 813, 814, 815, 816, 817, 818, 819, 820, 822, 824, 825, 826, 827, 829, 831, 832, 832, 834, 836, 838, 840, 841, 843, 845, 846, 847, 848, 849, 850, 851, 852, 854, 855, 856, 857, 858, 860, 861, 862, 863, 864, 867, 867, 868, 869, 870, 871, 872, 874, 876, 878, 880, 882, 884, 886, 888, 890, 892, 894, 896, 898, 899, 901, 903, 905, 906, 907, 909, 911, 913, 914, 915, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 975, 976, 977, 978, 979, 981, 982, 983, 985, 987, 989, 991, 993, 994, 995, and 996, wherein a plant produced from said plant cell has a difference in the level of salinity and/or oxidative stress tolerance as compared to the corresponding level of salinity and/or oxidative stress tolerance of a control plant that does not comprise said nucleic acid.

23. A transgenic plant comprising the plant cell of claim 21 or 22, wherein said polypeptide is selected from the group consisting of SEQ ID NO: 2, 4, 6, 7, 9, 11, 13, 15, 17, 19, 21, 23, 25, 27, 29, 30, 32, 33, 35, 37, 38, 40, 42, 44, 46, 48, 49, 51, 53, 54, 56, 58, 60, 62, 63, 64, 65, 66, 67, 68, 69, 71, 73, 75, 77, 79, 81, 83, 85, 87, 89, 91, 93, 95, 97, 98, 99, 100, 102, 103, 104, 106, 108, 110, 112, 114, 115, 116, 117, 118, 119, 120, 122, 124, 125, 127, 128, 129, 130, 132, 134, 136, 138, 140, 142, 144, 146, 147, 149, 151, 153, 154, 155, 157, 159, 161, 162, 163, 164, 165, 167, 169, 172, 174, 175, 178, 179, 180, 182, 184, 186, 187, 188, 189, 191, 192, 194, 196, 198, 200, 202, 204, 206, 208, 211, 217, 219, 223, 225, 226, 228, 230, 232, 233, 235, 237, 239, 241, 243, 245, 247, 249, 250, 251, 253, 255, 257, 259, 261, 262, 264, 266, 270, 271, 272, 273, 275, 279, 281, 282, 284, 286, 288, 290, 291, 292, 293, 294, 295, 297, 299, 301, 302, 304, 306, 308, 316, 318, 319, 320, 321, 323, 324, 331, 333, 335, 337, 339, 340, 344, 346, 348, 350, 351, 353, 355, 356, 357, 359, 361, 363, 365, 367, 369, 374, 375, 377, 378, 380, 382, 384, 386, 389, 392, 394, 396, 398, 400, 401, 402, 403, 405, 406, 407, 408, 410, 411, 412, 414, 416, 417, 418, 419, 421, 422, 424, 426, 428, 430, 431, 432, 433, 434, 436, 437, 438, 441, 442, 444, 445, 446, 447, 449, 450, 451, 452, 453, 454, 456, 457, 458, 459, 460, 461, 462, 463, 464, 465, 467, 468, 469, 470, 471, 472, 473, 474, 475, 477, 479, 480, 481, 482, 484, 486, 487, 489, 490, 491, 492, 493, 494, 496, 498, 500, 502, 503, 504, 505, 506, 507, 508, 509, 511, 512, 513, 514, 516, 517, 518, 519, 520, 522, 523, 524, 525, 527, 529, 531, 532, 533, 534, 536, 537, 538, 539, 540, 541, 543, 544, 546, 547, 549, 550, 551, 552, 553, 555, 556, 557, 559, 560, 562, 563, 564, 565, 566, 567, 569, 571, 572, 574, 576, 577, 578, 579, 582, 583, 585, 586, 588, 590, 591, 592, 593, 594, 595, 597, 598, 600, 601, 603, 605, 606, 608, 609, 610, 611, 613, 614, 615, 616, 617, 618, 620, 622, 624, 625, 626, 627, 629, 631, 633, 635, 637, 639, 640, 641, 642, 643, 644, 646, 648, 650, 651, 653, 654, 655, 657, 659, 660, 661, 662, 663, 664, 665, 666, 668, 669, 671, 673, 675, 677, 678, 680, 682, 684, 686, 688, 690 693, 694, 697, 699, 703, 706, 707, 709, 711, 712, 714, 718, 720, 722, 723, 724, 726, 728, 730, 732, 733, 735, 737, 739, 740 741, 742, 743, 745, 746, 747, 749, 751, 752, 753, 755, 757, 759, 761, 764, 765, 766, 767, 769, 770, 771, 773, 774, 775, 776, 777, 779, 780, 781, 782, 784, 785, 786, 788, 790, 792, 793, 795, 796, 797, 798, 801, 802, 804, 805, 808, 809, 810, 812, 813, 814, 815, 816, 817, 818, 819, 820, 822, 824, 825, 826, 827, 829, 831, 832, 834, 836 838, 840, 841, 843, 845, 846, 847, 848, 849, 850, 851, 852, 854, 855, 856, 857, 858, 860, 861, 862, 863, 864, 866, 867, 868, 869, 870, 871, 872, 874, 876, 878, 880, 882, 884, 886, 888, 890, 892, 894, 896, 898, 899, 901, 903, 905, 906 907, 909, 911, 913, 914, 915, 916, 918, 920, 922, 924, 926, 928, 930, 932, 934, 936, 938, 940, 942, 944, 946, 948, 950 952, 954, 956, 958, 960, 962, 964, 966, 968, 970, 972, 974, 975, 976, 977, 978, 979, 981, 982, 983, 985, 987, 989, 991, 993, 994, 995, and 996.

24. A plant cell comprising an exogenous nucleic acid said exogenous nucleic acid comprising a regulatory region operably linked to a nucleotide sequence having 80 percent or greater sequence identity to a fragment of a nucleotide sequence selected from the group consisting of SEQ ID NO: 1, 3, 5, 8, 10, 12, 14, 16, 18, 20, 22, 24, 26, 28, 31, 34, 36, 39, 41, 43, 45, 47, 50, 52, 55, 7, 59, 61, 70, 72, 74, 76, 78, 80, 82, 84, 86, 88, 90, 92, 94, 96, 101, 105, 107, 109, 111, 113, 121, 123, 126, 131, 133, 135, 137, 139, 141, 145, 148, 150, 152, 156, 158, 160, 166, 168, 170, 171, 173, 177, 181, 183, 185, 190, 193, 195, 197, 199, 201, 203, 205, 207, 210, 216, 218, 222, 224, 227, 229, 231, 234, 236, 238, 240, 242, 244, 246, 248, 252, 254, 256, 258, 260, 263, 265, 269, 274, 278, 280, 283, 285, 287, 289, 296, 298, 300, 303, 305, 307, 315, 317, 322, 330, 332, 334, 336, 338, 343, 345, 347, 349, 352, 354, 358, 360, 362, 364, 366, 368, 376, 379, 381, 383, 385, 387, 388, 391, 393, 395, 397, 399, 404, 409, 413, 415, 420, 423, 425, 427, 429, 435, 439, 440, 443, 448, 455, 466, 476, 478, 483, 485, 488, 495, 497, 499, 501, 510, 515, 521, 526, 528, 530, 535, 542, 545, 548, 554, 558, 561, 568, 570, 573, 575, 579, 581, 584, 587, 589, 596, 599, 602, 604, 607, 612, 619, 621, 623, 628, 630, 632, 634, 636, 638, 645, 647, 649, 652, 656, 658, 667, 670, 672, 674, 676, 679, 681, 683, 685, 687, 689, 691, 692, 695, 696, 698, 702, 708, 710, 713, 717, 719, 721, 725, 727, 729, 731, 734, 736, 738, 744, 748, 750, 754, 756, 758, 760, 762, 763, 768, 772, 778, 783, 787, 789, 791, 794, 799, 800, 803, 807, 811, 821, 823, 828, 830, 833, 835, 837, 839, 842, 844, 853, 859, 865, 873, 875, 877, 879, 881, 883, 885, 887, 889, 891, 893, 895, 897, 900, 902, 904, 908, 910, 912, 917, 919, 921, 923, 925, 927, 929, 931, 933, 935, 937, 939, 941, 943, 945, 947, 949, 951, 953, 955, 957, 959, 961, 963, 965, 967, 969, 971, 973, 980, 984, 986, 988, 990, and 992, wherein a plant produced from said plant cell has a difference in the level of salinity and/or oxidative stress tolerance as compared to the corresponding level of salinity and/or oxidative stress tolerance of a control plant that does not comprise said nucleic acid.

25. A transgenic plant comprising the plant cell of any one of claim 21, 22 or 24.

26. The transgenic plant of claim 25, wherein said plant is a member of a species selected from the group consisting of Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), or Pennisetum glaucum (pearl millet).

27. A seed product comprising embryonic tissue from a transgenic plant according to claim 26.

28. An isolated nucleic acid comprising a nucleotide sequence having 80% or greater sequence identity to the nucleotide sequence set forth in SEQ ID NO: 10, 12, 14, 16, 18, 20, 22, 24, 26, 28, 31, 34, 36, 39, 41, 43, 45, 52, 55, 57, 59, 61, 70, 72, 74, 76, 78, 80, 82, 84, 86, 88, 90, 92, 94, 96, 101, 105, 107, 109, 113, 121, 123, 126, 131, 133, 135, 139, 141, 145, 150, 152, 156, 158, 160, 166, 168, 170, 171, 173, 177, 181, 183, 185, 190, 193, 195, 197, 201, 203, 205, 207, 216, 218, 222, 224, 227, 229, 231, 234, 236, 240, 242, 244, 246, 248, 252, 254, 258, 260, 263, 265, 269, 274, 278, 280, 283, 285, 287, 289, 296, 298, 300, 303, 305, 307, 317, 322, 332, 334, 336, 338, 343, 345, 347, 349, 352, 354, 360, 362, 364, 366, 368, 376, 381, 383, 385, 391, 393, 397, 399, 404, 413, 415, 423, 425, 427, 429, 435, 443, 448, 455, 466, 476, 478, 483, 485, 495, 497, 499, 501, 510, 515, 521, 526, 528, 530, 535, 542, 545, 548, 554, 558, 561, 568, 570, 573, 575, 580, 581, 584, 587, 589, 596, 599, 602, 604, 607, 612, 619, 623, 630, 633, 634, 636, 638, 645, 647, 649, 652, 656, 658, 667, 670, 672, 674, 676, 679, 681, 683, 685, 687, 689, 691, 695, 698, 702, 708, 710, 713, 717, 719, 721, 729, 731, 734, 736, 738, 744, 748, 750, 754, 756, 758, 760, 768, 772, 778, 783, 787, 789, 791, 794, 799, 803, 811, 821, 823, 828, 830, 833, 835, 837, 839, 842, 844, 853, 859, 865, 873, 875, 877, 879, 881, 883, 885, 887, 889, 893, 895, 897, 900, 902, 904, 909, 910, 912, 917, 919, 921, 923, 925, 927, 933, 935, 937, 939, 941, 943, 945, 947, 949, 951, 953, 955, 957, 959, 961, 963, 965, 967, 969, 971, 973, 980, 984, 986, 988, 990, or 992.

29. An isolated nucleic acid comprising a nucleotide sequence encoding a polypeptide having 80% or greater sequence identity to the amino acid sequence set forth in SEQ ID NO: 11, 13, 15, 17, 19, 21, 23, 25, 27, 29, 32, 35, 37, 40, 42, 44, 46, 53, 56, 58, 60, 62, 71, 73, 75, 77, 79, 81, 83, 85, 87, 89, 91, 93, 95, 97, 102, 106, 108, 110, 114, 122, 124, 127, 132, 134, 136, 140, 142, 146, 151, 153, 157, 159, 161, 167, 169, 172, 174, 178, 182, 184, 186, 191, 194, 196, 198, 202, 204, 206, 208, 217, 219, 223, 225, 228, 230, 232, 235, 237, 241, 243, 245, 247, 249, 253, 255, 259, 261, 264, 266, 270, 275, 279, 281, 284, 286, 288, 290, 297, 299, 301, 304, 306, 308, 318, 323, 333, 335, 337, 339, 344, 346, 348, 350, 353, 355, 361, 363, 365, 367, 369, 377, 382, 384, 386, 392, 394, 398, 400, 405, 414, 416, 424, 426, 428, 430, 436, 444, 449, 456, 467, 477, 479, 484, 486, 496, 498, 500, 502, 511, 516, 522, 527, 529, 531, 536, 543, 546, 549, 555, 559, 562, 569, 571, 574, 576, 580, 582, 585, 588, 590, 597, 600, 603, 605, 608, 613, 620, 624, 631, 634, 635, 637, 639, 646, 648, 650, 653, 657, 659, 668, 671, 673, 675, 677, 680, 682, 684, 686, 688, 690, 692, 696, 699, 703, 709, 711, 714, 718, 720, 722, 730, 732, 735, 737, 739, 745, 749, 751, 755, 757, 759, 761, 769, 773, 779, 784, 788, 790, 792, 795, 800, 804, 812, 822, 824, 829, 831, 834, 836, 838, 840, 843, 845, 854, 860, 866, 874, 876, 878, 880, 882, 884, 886, 888, 890, 894, 896, 898, 901, 903, 905, 910, 911, 913, 918, 920, 922, 924, 926, 928, 934, 936, 938, 940, 942, 944, 946, 948, 950, 952, 954, 956, 958, 960, 962, 964, 966, 968, 970, 972, 974, 981, 985, 987, 989, 991, or 993.

30. A method of identifying whether a polymorphism is associated with variation in a trait, said method comprising:
a) determining whether one or more genetic polymorphisms in a population of plants is associated with the locus for a polypeptide selected from the group consisting of the polypeptides depicted in FIGS. 1-16, a polypeptide having an amino acid sequence of SEQ ID NO: 692, and functional homologs thereof; and
b) measuring the correlation between variation in said trait in plants of said population and the presence of said one or more genetic polymorphisms in plants of said population, thereby identifying whether or not said one or more genetic polymorphisms are associated with variation in said trait.

31. A method of making a plant line, said method comprising:
a) determining whether one or more genetic polymorphisms in a population of plants is associated with the locus for a polypeptide selected from the group consisting of the polypeptides depicted in FIGS. 1-16, a polypeptide having an amino acid sequence of SEQ ID NO: 692, and functional homologs thereof;
b) identifying one or more plants in said population in which the presence of at least one allele at said one or more genetic polymorphisms is associated with variation in a salinity and/or oxidative stress tolerance trait;
c) crossing each said one or more identified plants with itself or a different plant to produce seed;
d) crossing at least one progeny plant grown from said seed with itself or a different plant; and
e) repeating steps c) and d) for an additional 0-5 generations to make said plant line, wherein said at least one allele is present in said plant line.

32. The method of claim 30 or 31, wherein said trait is the level of salinity and/or oxidative stress tolerance.

33. The method of claim 30 or 31, wherein said population is a population of switchgrass, sorghum, sugar cane, or miscanthus plants.
